(19) United States
(12) Patent Application Publication
(10) Pub. No.: US 2022/0054632 A1
(43) Pub. Date: Feb. 24, 2022

(54) MODIFIED CARRIER PROTEINS FOR O-LINKED GLYCOSYLATION

(71) Applicant: GLAXOSMITHKLINE BIOLOGICALS SA, Rixensart (BE)

(72) Inventors: Rainer FOLLADOR, Schlieren (CH); Stefan Jochen KEMMLER, Schlieren (CH); Michael Thomas KOWARIK, Schlieren (CH); Gerald Johann POSCH, Schlieren (CH)

(73) Assignee: GLAXOSMITHKLINE BIOLOGICALS SA, Rixensart (BE)

(21) Appl. No.: 17/299,347

(22) PCT Filed: Dec. 11, 2019
(86) PCT No.: PCT/EP2019/084632

§ 371 Date: Jun. 3, 2021

(30) Foreign Application Priority Data
Dec. 12, 2018 (EP) 18212100.4

(51) Int. Cl. A61K 39/385; C07K 14/22; C07K 14/28; C07K 14/33; C07K 14/34; C07K 14/21; C07K 14/205; C07K 14/245; C12N 15/70

(52) U.S. Cl. CPC A61K 39/385; C07K 14/22; C07K 14/28; C07K 14/33; A61K 2039/6068; C07K 14/21; C07K 14/205; C07K 14/245; C12N 15/70; C07K 14/34

(57) ABSTRACT

Carrier proteins modified to incorporate one or more pilin glycotags and applications thereof for O-linked glycosylation are provided. In particular, a modified carrier protein comprising a carrier protein that comprises at least one GlycoTag, wherein the at least one GlycoTag is a Neisseria gonorrhoeae PglL GlycoTag (NgGlycoTag), Neisseria lactamica PglL GlycoTag (NlGlycoTag), or Neisseria shayeganii GlycoTag (NsGlycoTag), or combinations thereof is provided, together with nucleic acids and vectors encoding the modified carrier protein, host cells comprising these modofoed carrier proteins or nucleic acids encoding them, bioconjugates, methods of making bioconjugates and uses of the bioconjugates.

### REFERENCE TO SEQUENCE LISTING

The instant application contains an electronically submitted Sequence Listing in ASCII text file format which is hereby incorporated by reference in its entirety.

### FIELD OF THE INVENTION

The field of the invention generally relates to modified carrier proteins comprising one or more GlycoTags and the use of such modified carrier proteins in efficient O-linked glycosylation, for example using PglL.

### BACKGROUND OF THE INVENTION

Protein glycosylation is a common posttranslational modification in bacteria by which glycans are covalently attached to surface proteins, flagella, or pili, for example. [1]. Glycoproteins play roles in adhesion, stabilization of proteins against proteolysis, and evasion of the host immune response. [1]. Two protein glycosylation mechanisms are distinguished by the mode in which the glycans are transferred to proteins: one mechanism involves the transfer of carbohydrates directly from nucleotide-activated sugars to acceptor proteins (used in, e.g., protein O-glycosylation in the Golgi apparatus of eukaryotic cells and flagellin O-glycosylation in some bacteria). A second mechanism involves the preassembly of a polysaccharide onto a lipid-carrier (by glycosyltransferases) which is then transferred to a protein acceptor by an oligosaccharyltransferase (OTase). [1]. This second mechanism is used in, e.g., N-glycosylation in the endoplasmic reticulum of eukaryotic cells, the well-characterized N-linked glycosylation system of Campylobacter jejuni, and the more recently characterized O-linked glycosylation systems of Neisseria meningitidis, Neisseria gonococcus, and Pseudomonas aeruginosa. [1]. For O-linked glycosylation (O-glycosylation), glycans are generally attached to a serine or threonine residue on the protein acceptor. For N-linked glycosylation (N-glycosylation), glycans are generally attached to an asparagine residue on the protein acceptor. See generally [2].

The two best understood glycosylation systems are the C. jejuni N-linked glycosylation system and the Neisseria O-linked glycosylation system. [1], [3]. In these two systems, a polysaccharide (glycan donor) linked to an undecaprenyl pyrophosphate (UndPP) lipid-carrier is translocated (flipped) to the periplasm by a flippase. [2], [3]. In the periplasm, an oligosaccharyltransferase (OTase) transfers the glycan to a protein acceptor (pilin). [2], [3]. The OTase of C. jejuni (PglB) transfers the glycan to the asparagine (N) in the conserved pilin pentapeptide motif D/E-X₁-N-X₂-S/T (where X₁ and X₂ are any residues except proline). [4]. The OTase of N. meningitidis (NmPglL) transfers the glycan to Ser63 in the N. meningitidis pilin PilE sequence (“sequon”) (N)-SAVTEYYLNHGEWPGNNTSAGVATSSEIK-(C) (SEQ ID NO: 140, corresponding to residues 45-73 of mature N. meningitidis PilE sequence SEQ ID NO: 137). [1], [3], [5]. Until this disclosure, the pilin sequence onto which other OTases (from N. gonorrhoeae, N. lactamica, or N. shayeganii for example) transfer glycan was not known (see [6]).

Conjugate vaccines (comprising a carrier protein covalently linked to an immunogenic glycan) have been a successful approach for vaccination against a variety of bacterial infections. However, the chemical methods by which they are routinely produced are complex and comparatively inefficient ([4] at FIG. 1). To increase conjugate vaccine production efficiency, in vivo methods (hence “bioconjugate vaccine”) have been in development. These in vivo methods leverage the N-glycosylation and O-glycosylation systems discussed above, particularly the OTase sequons, so that proteins which are not otherwise glycosylated by the OTase (carrier proteins), are glycosylated in vivo.

For example, carrier proteins AcrA and EPA were N-glycosylated in E. coli using heterologous polysaccharide as glycan donors and C. jejuni PglB because AcrA and EPA were first modified to incorporate an appropriate periplasmic signal sequence and at least one copy of the PglB sequon sequence D/E-X₁-N-X₂-S/T (a “GlycoTag”). [4]; see also [7], [8], [9], [10], [11] (all of which are incorporated herein by reference in their entireties). The use of PglB-based bioconjugation production is limited because PglB only accepts certain sugar substrates: those containing an acetamido group at position C-2 of the reducing end and those that do not possess a β1, 4 linkage between the first two sugars (i.e., the linkage between sugars “S-2” and “S-1”, the first sugar (S-1) comprising the reducing end and S-2 being adjacent to S-1). [3], [12], [13].

To overcome this limitation of PglB-based systems and because Neisserial PglLs are “promiscuous” with respect to sugar substrates ([3]), an O-glycosylation system using the PglL OTase from Neisseria meningitidis has been the focus of recent work ([1], [14], [15], [16]; see also [6]).

For example, carrier proteins EPA, TTc, and CTB were O-glycosylated by N. meningitidis PglL in Shigella flexneri using polysaccharides which were endogenous to the Shigella flexneri host cell as glycan donors (“endogenous polysaccharide”) because each carrier protein was modified to incorporate a periplasmic signal sequence and one copy of the N. meningitidis PilE sequon sequence
(N)-SAVTEYYLNHGEWPGNNTSAGVATSSEIK-(C) (SEQ ID NO: 140)
   (EPA and TTc modified at their N-terminuses, CTB modified at C-terminus). [3]. O-glycosylation was also achieved for those modified EPA and CTB carrier proteins in E. coli and Salmonella enterica using NmPglL and endogenous polysaccharides. [3]. Smaller NmGlycoTags were also demonstrated (all fragments of the sequence SEQ ID NO: 140), the smallest being 12 amino acids in length (successfully used if two hydrophilic fragments flanked it) ([3] at 6).

But like its predecessor, the applicability of this NmPglL work is limited at least because only O-glycosylation by NmPglL paired with NmPilE sequon sequences was demonstrated and the system showed an unfortunate bias toward CTB as carrier protein (CTB was more effective than the desirable carrier protein, EPA). [3]; see also [5].

An array of PglL OTases and pilin sequons are needed that may be optimally paired for efficient O-glycosylation of a variety of carrier proteins, especially EPA, and at internal glycosylation sites.

### SUMMARY OF THE INVENTION

In one aspect, the present invention is the first to describe certain pilin sequences and modified carrier proteins comprising them, optionally wherein the pilin sequence is O-glycosylated by an OTases from NmPglL or a homologue thereof (such as OTases from N. gonorrhoeae, N. lactamica, or N. shayeganii). In another aspect, the present invention provides efficient O-glycosylation of a variety of glycotagged carrier proteins, especially EPA, and with GlycoTags located at N-terminal, C-terminal, and/or internal carrier protein residues (internal GlycoTags being expected to improve conjugate characteristics such as stability over time).

Embodiments of the present invention include, but are not limited to:
1. A modified carrier protein comprising a carrier protein that comprises at least one GlycoTag, wherein the at least one GlycoTag is a Neisseria gonorrhoeae PglL GlycoTag (NgGlycoTag), Neisseria lactamica PglL GlycoTag (NlGlycoTag), or Neisseria shayeganii GlycoTag (NsGlycoTag), or combinations thereof.
2. The modified carrier protein of embodiment 1, wherein the at least one GlycoTag is located at the N-terminus, C-terminus, and/or interior of the carrier protein.
3. The modified carrier protein of embodiment 1 or 2, wherein the at least one NgGlycoTag consists of a peptide sequence that is 12 to 30 amino acids long and comprises therein the sequence SEQ ID NO: 147. For example the NgGlycoTag is 12, 13, 14, 15, 16, 17, 18, 19, 20, 21, 22, 23, 24, 25, 26, 27, 28, 29 or 30 amino acids long or 13-29, 14-28, 15-27, 16-25, 17-24, 18-13, 12-25, 12-20 or 12-15 amino acids long. In an embodiment 1, 2, 3, 4 or 5 or 1-5, 1-4, 1-3 or 1-2 amino acid substitution(s), where the amino acid is altered from the amino acid at that position of SEQ ID NO: 147 is present in the NgGlycoTag amino acid sequence. In an embodiment, the amino acid substitution(s) is a conservative substitution(s).
4. The modified carrier protein of embodiment 1, 2, or 3, wherein the at least one NgGlycoTag consists of a peptide sequence that is 30 amino acids long and comprises therein the sequence SEQ ID NO: 147.
5. The modified carrier protein of embodiment 4, wherein the at least one NgGlycoTag consists of a peptide sequence that is SEQ ID NO: 145. In an embodiment 1, 2, 3, 4 or 5 or 1-5, 1-4, 1-3 or 1-2 amino acid substitution(s), where the amino acid is altered from the amino acid at that position of SEQ ID NO: 145 is present in the NgGlycoTag amino acid sequence. In an embodiment, the amino acid substitution(s) is a conservative substitution(s).
6. The modified carrier protein of embodiment 1, 2, or 3, wherein the at least one NgGlycoTag consists of a peptide sequence that is 20 amino acids long and comprises therein the sequence SEQ ID NO: 147.
7. The modified carrier protein of embodiment 6, wherein the at least one NgGlycoTag consists of a peptide sequence that is SEQ ID NO: 146.
8. The modified carrier protein of embodiment 3, wherein the at least one NgGlycoTag consists of a peptide sequence that is SEQ ID NO: 147.
   In certain embodiments, the modified carrier protein has the amino acid sequence of one of SEQ ID NOs: 101, 113, 115, 117, 119, 121, 123, 125, 127, 129, 131, and 133.
9. The modified carrier protein of embodiment 1 or 2, wherein the at least one NlGlycoTag consists of a peptide sequence that is 12 to 35 amino acids long and comprises therein the sequence SEQ ID NO: 151. For example the NlGlycoTag is 12, 13, 14, 15, 16, 17, 18, 19, 20, 21, 22, 23, 24, 25, 26, 27, 28, 29 or 30 amino acids long or 13-29, 14-28, 15-27, 16-25, 17-24, 18-13, 12-25, 12-20 or 12-15 amino acids long. In an embodiment 1, 2, 3, 4 or 5 or 1-5, 1-4, 1-3 or 1-2 amino acid substitution(s), where the amino acid is altered from the amino acid at that position of SEQ ID NO:151 is present in the NlGlycoTag amino acid sequence. In an embodiment, the amino acid substitution(s) is a conservative substitution(s).
10. The modified carrier protein of embodiment 9, wherein the at least one NlGlycoTag consists of a peptide sequence that is 35 amino acids long and comprises therein the sequence SEQ ID NO: 151.
11. The modified carrier protein of embodiment 9 or 10, wherein the at least one NlGlycoTag consists of a peptide sequence that is SEQ ID NO: 150.
12. The modified carrier protein of embodiment 9, wherein the at least one NlGlycoTag consists of a peptide sequence that is SEQ ID NO: 151.
   In certain embodiments, the modified carrier protein has the amino acid sequence SEQ ID NO: 103.
13. The modified carrier protein of embodiment 1 or 2, wherein the at least one NsGlycoTag consists of a peptide sequence that is 12 to 31 amino acids long and comprises therein the sequence SEQ ID NO: 164. For example the NsGlycoTag is 12, 13, 14, 15, 16, 17, 18, 19, 20, 21, 22, 23, 24, 25, 26, 27, 28, 29 or 30 amino acids long or 13-29, 14-28, 15-27, 16-25, 17-24, 18-13, 12-25, 12-20 or 12-15 amino acids long. In an embodiment 1, 2, 3, 4 or 5 or 1-5, 1-4, 1-3 or 1-2 amino acid substitution(s), where the amino acid is altered from the amino acid at that position of SEQ ID NO:164 is present in the NsGlycoTag amino acid sequence. In an embodiment, the amino acid substitution(s) is a conservative substitution(s).
14. The modified carrier protein of embodiment 13 wherein the at least one NsGlycoTag consists of a peptide sequence that is 31 amino acids long and comprises therein the sequence SEQ ID NO: 164.
15. The modified carrier protein of embodiment 13 or 14, wherein the at least one NsGlycoTag consists of a peptide sequence that is SEQ ID NO: 163.
16. The modified carrier protein of embodiment 13, wherein the at least one NsGlycoTag consists of a peptide sequence that is SEQ ID NO: 164.
17. The modified carrier protein of one of embodiments 1 to 16, further comprising at least one Neisseria meningitidis PglL GlycoTag (NmGlycoTag).
   In certain embodiments, the modified carrier protein has the amino acid sequence SEQ ID NO: 111.
18. A modified carrier protein comprising a carrier protein that comprises at least one GlycoTag, wherein the at least one GlycoTag is a Neisseria meningitidis PglL GlycoTag (NmGlycoTag) consisting of a peptide sequence that is 12 to 19 amino acids long and comprises therein the sequence 142. For example the NmGlycoTag is 12, 13, 14, 15, 16, 17, 18, 19, 20, 21, 22, 23, 24, 25, 26, 27, 28, 29 or 30 amino acids long or 13-29, 14-28, 15-27, 16-25, 17-24, 18-13, 12-25, 12-20 or 12-15 amino acids long. In an embodiment 1, 2, 3, 4 or 5 or 1-5, 1-4, 1-3 or 1-2 amino acid substitution(s), where the amino acid is altered from the amino acid at that position of SEQ ID NO: 142 is present in the NmGlycoTag amino acid sequence. In an embodiment, the amino acid substitution(s) is a conservative substitution(s).
19. The modified carrier protein of embodiment 18, wherein the at least one NmGlycoTag consists of a peptide sequence that is 19 amino acids long and comprises therein the sequence SEQ ID NO: 142.
20. The modified carrier protein of embodiment 18 or 19, wherein the at least one NmGlycoTag consists of a peptide sequence that is SEQ ID NO: 141.
21. The modified carrier protein of embodiment 18, wherein the at least one NmGlycoTag consists of a peptide sequence that is SEQ ID NO: 142.
   Certain embodiments are provided with the proviso that the NmGlycoTag does not consist of the sequence SEQ ID NO: 140.
   In certain embodiments, the modified carrier protein has the amino acid sequence of one of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 199, 202, and 204.
22. The modified carrier protein of one of embodiments 1-21, wherein the carrier protein is selected from the group consisting of cholera toxin b subunit (CTB), tetanus toxoid (TT), tetanus toxin C fragment (TTc), diphtheria toxoid (DT), CRM197, Pseudomonas aeruginosa exotoxin A (EPA), C. jejuni Acriflavine resistance protein A (CjAcrA), E. coli Acriflavine resistance protein A (EcAcrA), and Pseudomonas aeruginosa PcrV (PcrV).
23. The modified carrier protein of one of embodiments 1-22, wherein the carrier protein is EPA.
24. The modified carrier protein of embodiment 23, wherein the at least one GlycoTag is located at, numbered with respect to SEQ ID NO: 1, residue A14, D36, Q92, G123, E157, A177, Y208, N231, E252, R274, A301, Q307, A365, S408, T418, A464, A519, G525, A533, S585, K240, or A375, or combinations thereof. In an embodiment, residue A14, D36, Q92, G123, E157, A177, Y208, N231, E252, R274, A301, Q307, A365, S408, T418, A464, A519, G525, A533, S585, K240, or A375, or combinations thereof is/are substituted with the at least one GlycoTag.
25. A modified carrier protein, characterized by a Pseudomonas aeruginosa exotoxin A (EPA) carrier protein comprising at least one Neisseria meningitidis PglL GlycoTag (NmGlycoTag), wherein the at least one NmGlycoTag is located at, with respect to SEQ ID NO: 1, residue A14, D36, Q92, G123, E157, A177, Y208, N231, E252, R274, A301, Q307, A365, S408, T418, A464, A519, G525, A533, S585, K240, or A375, or combinations thereof. In an embodiment, residue A14, D36, Q92, G123, E157, A177, Y208, N231, E252, R274, A301, Q307, A365, S408, T418, A464, A519, G525, A533, S585, K240, or A375, or combinations thereof are substituted with the NmGlycoTag
26 The modified carrier protein of embodiment 25, wherein the at least one NmGlycoTag consists of a peptide sequence that is 12 to 29 amino acids long and comprises therein the sequence SEQ ID NO: 142.
27. The modified carrier protein of embodiment 25 or 26, wherein the at least one NmGlycoTag consists of a peptide sequence that is 29 amino acids long and comprises therein the sequence SEQ ID NO: 142.
28. The modified carrier protein of embodiment 25, 26, or 27, wherein the at least one NmGlycoTag consists of a peptide sequence that is SEQ ID NO: 140.
29. The modified carrier protein of embodiment 25 or 26, wherein the at least one NmGlycoTag consists of a peptide sequence that is 19 amino acids long and comprises therein the sequence SEQ ID NO: 142.
30. The modified carrier protein of one of embodiment 25-29, wherein the at least one NmGlycoTag consists of a peptide sequence that is SEQ ID NO: 141.
31. The modified carrier protein of embodiment 25 or 26, wherein the at least one NmGlycoTag consists of a peptide sequence that is SEQ ID NO: 142.
32. The modified carrier protein of embodiment 25, comprising at least a second GlycoTag located at the N-terminus, C-terminus, and/or interior of the carrier protein.
33. The modified carrier protein of embodiment 32, comprising two or more GlycoTags and wherein at least the second GlycoTag is a NgGlycoTag, NlGlycoTag, or NsGlycoTag.
   Certain embodiments are provided with the proviso that the NmGlycoTag does not consist of the sequence SEQ ID NO: 140.
   In certain embodiments, the modified carrier protein has the amino acid sequence of one of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, and 135.
34. The modified carrier protein of one of embodiments 1-33, further comprising one or more GlycoTag-flanking Peptide (G-f Peptide).
35. The modified carrier protein of one of embodiment 1-34, wherein the one or more G-f Peptide is located at the N-terminus, C-terminus, or combinations thereof, of a GlycoTag.
36. The modified carrier protein of one of embodiment 1-34, wherein the one or more G-f Peptide is adjacent to a GlycoTag.
   The modified carrier protein of any one of embodiments 1-36, wherein the modified carrier protein is coupled to (optionally covalently coupled to) a glycan at one or more of the GlycoTags.
      The modified carrier protein above, wherein the glycan is a PglL Glycan Substrate.
      The modified carrier protein above, wherein the glycan has a reducing end structure of
      (i) a reducing end structure of Glucose, Galactose, Galactofuranose, Rhamnose, GlcNAc, GalNAc, FucNAc, DATDH, GATDH, HexNAc, deoxy HexNAc, diNAcBac, or Pse;
      (ii) a reducing end structure of DATDH, GlcNAc, GalNAc, FucNAc, Galactose, or Glucose;
      (iii) a reducing end structure of GlcNAc, GalNAc, FucNAc, or Glucose; or
      (iv) a S-2 to S-1 reducing end structure of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine.
      The modified carrier protein above, wherein the glycan is a
         Shigella glycan (e.g. a Shigella sonnei glycan (such as S. sonnei O-antigen), or a Shigella flexneri glycan (such as Shigella flexneri 2a CPS), or a Shigella dysenteriae glycan)
         or a Streptococcus glycan (e.g. Streptococcus pneumoniae (such as Streptococcus pneumoniae sp. 12F CPS, S. pneumoniae sp. 8 CPS, S. pneumoniae sp. 14 CPS, S. pneumoniae sp. 23A CPS, S. pneumoniae sp. 33F CPS, or S. pneumoniae sp. 22A CPS).
      The modified carrier protein above, wherein (a) the glycan is a
         Shigella glycan (e.g. a Shigella sonnei glycan (such as S. sonnei O-antigen), or a Shigella flexneri glycan (such as Shigella flexneri 2a CPS), or a Shigella dysenteriae glycan)
         or a Streptococcus glycan (e.g. Streptococcus pneumoniae (such as Streptococcus pneumoniae sp. 12F CPS, S. pneumoniae sp. 8 CPS, S. pneumoniae sp. 14 CPS, S. pneumoniae sp. 23A CPS, S. pneumoniae sp. 33F CPS, or S. pneumoniae sp. 22A CPS); and
         (b) the glycan has a reducing end structure of
            (i) a reducing end structure of Glucose, Galactose, Galactofuranose, Rhamnose, GlcNAc, GalNAc, FucNAc, DATDH, GATDH, HexNAc, deoxy HexNAc, diNAcBac, or Pse;
            (ii) a reducing end structure of DATDH, GlcNAc, GalNAc, FucNAc, Galactose, or Glucose;
            (iii) a reducing end structure of GlcNAc, GalNAc, FucNAc, or Glucose; or
            (iv) a S-2 to S-1 reducing end structure of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine.
      The modified carrier protein above, wherein (a) the glycan is a Streptococcus glycan (e.g. Streptococcus pneumoniae (such as S. pneumoniae sp. 8 CPS, Streptococcus pneumoniae sp. 12F CPS, S. pneumoniae sp. 14 CPS, S. pneumoniae sp. 22A CPS, S. pneumoniae sp. 23A CPS, or S. pneumoniae sp. 33F CPS); and (b) the glycan has a S-2 to S-1 reducing end structure of Glucuronic acid-β1,4-glucose, N-acetyl-fucosamine-α1,3-N-acetyl-galactosamine, Galactose-β1,4-glucose, Rhamnose-β1,4-glucose, or Galactofuranose-β1,3-glucose.
37. A nucleic acid molecule comprising a nucleotide sequence that encodes the modified carrier protein of any one of the above embodiments or any one of embodiments 1-36.
38. The nucleic acid molecule of embodiment 37, wherein the nucleotide sequence is codon optimized for expression within a Neisseria, Shigella, Salmonella, Escherichia, Pseudomonas, Yersinia, Campylobacter, or Heliobacter cell.
39. A vector comprising the nucleic acid molecule of embodiments 37 or 38 and wherein the modified carrier protein nucleotide sequence is operatively linked to a polynucleotide sequence encoding a periplasmic signal sequence.
40. The vector of embodiment 39, further comprising a nucleic acid molecule that comprises a nucleotide sequence encoding a Neisseria meningitidis PglL (NmPglL) Oligosaccharyltransferase (OTase), Neisseria gonorrhoeae PglL (NgPglL) OTase, Neisseria lactamica 020-06 (NlPglL) OTase, Neisseria lactamica ATCC 23970 PglL (Nl_{ATCC23970}PglL) OTase, or Neisseria gonorrhoeae F62 PglL (Ng_{F62}PglL) OTase.
   In certain embodiments, the vector is an expression vector.
   In certain embodiments, the vector further comprises a nucleic acid molecule that comprises a nucleotide sequence encoding a Neisseria meningitidis PglL, Neisseria gonorrhoeae PglL, Neisseria lactamica 020-06 PglL, Neisseria lactamica ATCC 23970 PglL, Neisseria gonorrhoeae F62 PglL, Neisseria cinerea ATCC 14685 PglL, Neisseria mucosa PglL, Neisseria flavescens NRL30031/H210 PglL, Neisseria mucosa ATCC 25996 PglL, Neisseria sp. oral taxon 014 strain F0314 PglL, Neisseria arctica PglL, Neisseria shayeganii 871 PglL, Neisseria shayeganii 871 PglL, Neisseria sp. 83E34 PglL, Neisseria wadsworthii PglL, Neisseria elongata subsp. glycolytica ATCC 29315 PglL, Neisseria bacillformis ATCC BAA-1200 PglL, Neisseria sp. oral taxon 020 str. F0370 PglL, Neisseria sp. 74A18 PglL, Neisseria weaver ATCC 51223 PglL, or Neisseria macacae ATCC 33926 PglL OTase.
   In certain embodiments, the vector further comprises a nucleic acid molecule that comprises the nucleotide sequence of one of SEQ ID NOs: 8, 10, 12, 14, 16, 18, 20, 22, 24, 26, 28, 30, 32, 34, 36, 38, 40, 42, 44, 46, and 48.
   In certain embodiments, the vector further comprises a nucleic acid molecule that comprises a nucleotide sequence encoding the amino acid sequence SEQ ID NO: 9, 11, 13, 15, 17, 19, 21, 23, 25, 27, 29, 31, 33, 35, 37, 39, 41, 43, 45, 47, or 49.
41. A gram-negative bacterial host cell comprising the vector of embodiments 39 or 40.
42. The host cell of embodiment 41 which is a Neisseria, Salmonella, Shigella, Escherichia, Pseudomonas, or Yersinia cell.
43. A gram-negative bacterial cell, comprising one or more nucleic acid molecules that encode
   (a) a PglL Glycan Substrate;
   (b) Glycosyltransferases capable of assembling the PglL Glycan Substrate onto a lipid carrier;
   (c) a modified carrier protein as in one of embodiments 1-36, targeted to the periplasm; and
   (d) a PglL OTase.
44. The gram-negative bacterial cell of embodiment 43, wherein the cell comprises the one or more nucleic acid molecules in the nuclear DNA.
45. The gram-negative bacterial cell of embodiment 43 or 44, which is a Neisseria, Salmonella, Shigella, Escherichia, Pseudomonas, or Yersinia cell.
46 The gram-negative bacterial cell of embodiment 43, 44, or 45, wherein the PglL OTase is an endogenous PglL homologue.
47. The gram-negative bacterial cell of embodiment 43, 44, or 45, wherein the PglL OTase is heterologous to the cell and the cell's endogenous PglL homologue is reduced as compared to control.
48. A gram-negative bacterial cell, comprising at the periplasm:
   (a) a lipid-Carrier-Linked PglL Glycan Substrate,
   (b) a modified carrier protein as in any one of embodiments 1-36, and
   (c) a PglL OTase.
49. The gram-negative bacterial cell of embodiment 48 which is a Neisseria, Salmonella, Shigella, Escherichia, Pseudomonas, or Yersinia cell.
   In certain embodiments, the PglL Glycan Substrate is endogenous to a Neisseria, Shigella, Salmonella, Streptococcus, Escherichia, Pseudomonas, Yersinia, Campylobacter, or Heliobacter cell.
   In certain embodiments, the PglL Glycan Substrate is an O-antigen. In certain embodiments, the O-antigen is S. sonnei O-antigen.
   In certain embodiments, the PglL Glycan Substrate has a reducing end structure of Glucose, Galactose, Galactofuranose, Rhamnose, GlcNAc, GalNAc, FucNAc, DATDH, GATDH, HexNAc, deoxy HexNAc, diNAcBac, or Pse. In further embodiments, the lipid-linked PglL Glycan Substrate has a reducing end structure of DATDH, GlcNAc, GalNAc, FucNAc, Galactose, or Glucose. In further embodiments, the lipid-linked PglL Glycan Substrate has a reducing end structure of GlcNAc, GalNAc, FucNAc, or Glucose. In further embodiments, the PglL Glycan Substrate has a S-2 to S-1 reducing end structure of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine. In further embodiments, the PglL Glycan Substrate is a Shigella sonnei glycan antigen e.g. S. sonnei O-antigen, a Shigella flexneri glycan antigen e.g. Shigella flexneri 2a CPS, a Shigella dysenteriae glycan antigen, a Streptococcus pneumoniae glycan antigen e.g. Streptococcus pneumoniae sp. 12F CPS, S. pneumoniae sp. 8 CPS, S. pneumoniae sp. 14 CPS, S. pneumoniae sp. 15A CPS, S. pneumoniae sp. 33F CPS, or S. pneumoniae sp. 22A CPS.
   In certain embodiments, the PglL OTase is a Neisseria meningitidis PglL, Neisseria gonorrhoeae PglL, Neisseria lactamica 020-06 PglL, Neisseria lactamica ATCC 23970 PglL, Neisseria gonorrhoeae F62 PglL, Neisseria cinerea ATCC 14685 PglL, Neisseria mucosa PglL, Neisseria flavescens NRL30031/H210 PglL, Neisseria mucosa ATCC 25996 PglL, Neisseria sp. oral taxon 014 strain F0314 PglL, Neisseria arctica PglL, Neisseria shayeganii 871 PglL, Neisseria shayeganii 871 PglL, Neisseria sp. 83E34 PglL, Neisseria wadsworthii PglL, Neisseria elongata subsp. glycolytica ATCC 29315 PglL, Neisseria bacillformis ATCC BAA-1200 PglL, Neisseria sp. oral taxon 020 str. F0370 PglL, Neisseria sp. 74A18 PglL, Neisseria weaver ATCC 51223 PglL, or Neisseria macacae ATCC 33926 PglL OTase.
   In certain embodiments, the PglL Otase is a Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacillformis ATCC BAA-1200 (NbPglL) PglL Otase.
   In certain embodiments, the PglL Otase is a Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), and Neisseria bacillformis ATCC BAA-1200 (NbPglL), Neisseria mucosa ATCC 25996 (NmuPglL), or Neisseria shayeganii 871 (SEQ ID NO: 33, NsPglL) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC2397PglL}), or Neisseria gonorrhoeae F62 PglL (Ng_{F62PglL}) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase.
   In certain embodiments, the modified carrier protein has the amino acid sequence of one of SEQ ID NOs: 51, 53, and 55. In certain embodiments, the PglL Glycan Substrate has a reducing end structure of N-acetyl-fucosamine (FucNAc) and the modified carrier protein has the amino acid sequence SEQ ID NO: 51, 53, or 55. In certain embodiments, the PglL Glycan Substrate has a S-2 to S-1 reducing end structure of N-acetyl-altruronic acid-α1,3-4-amino-N-acetyl-fucosamine and the modified carrier protein has the amino acid sequence SEQ ID NO: 51, 53, or 55. In a further embodiment, the PglL Glycan Substrate is S. sonnei O-antigen. In a further embodiment, the PglL Otase is NmPglL.
   In certain embodiments, the modified carrier protein has the amino acid sequence of SEQ ID NO: 134 or 135. In certain embodiments, the PglL Glycan Substrate has a reducing end structure of GalNAc, FucNAc, or GlcNAc and the modified carrier protein has the amino acid sequence SEQ ID NO: 134 or 135. In certain embodiments, the PglL Glycan Substrate has a reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine and the modified carrier protein has the amino acid sequence SEQ ID NO: 134 or 135. In a further embodiment, the PglL Glycan Substrate is a Shigella (e.g., S. sonnei or S. flexneri) or Streptococcus (e.g., S. pneumonieae) antigen with a reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine and the modified carrier protein has the amino acid sequence SEQ ID NO: 134 or 135. In a further embodiment, the PglL Glycan Substrate is S. sonnei O-antigen, Shigella flexneri 2a CPS, or Streptococcus pneumoniae sp. 12F CPS. In a further embodiment, the PglL Otase is NmPglL.
   In a certain embodiment, the modified carrier protein has the amino acid sequence SEQ ID NO: 51, 199, 202, or 204. In a certain embodiment, the PglL Glycan Substrate has a reducing end structure of GlcNAc, GalNAc, FucNAc, or Glucose and the modified carrier protein has the amino acid sequence SEQ ID NO: 51, 199, 202, or 204. In a further embodiment, the PglL Glycan Substrate has a S-2 to S-1 reducing end structure of Galactose-β1,4-Glucose; Glucuronic acid-β1,4-glucose; N-acetyl-fucosamine-α1,3-N-acetyl-galactosamine; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine; and the modified carrier protein has the amino acid sequence SEQ ID NO: 51, 199, 202, or 204. In further embodiments, the PglL Glycan Substrate is a Shigella sonnei glycan antigen (e.g. S. sonnei O-antigen), a Shigella flexneri glycan antigen (e.g. Shigella flexneri 2a CPS), a Shigella dysenteriae glycan antigen, a Streptococcus pneumoniae glycan antigen (e.g. Streptococcus pneumoniae sp. 12F CPS, S. pneumoniae sp. 8 CPS, S. pneumoniae sp. 14 CPS, S. pneumoniae sp. 15A CPS, or S. pneumoniae sp. 33F CPS). In a further embodiment, the PglL Otase is NmPglL.
   In certain embodiments, the PglL Glycan Substrate has a reducing end structure of N-acetyl-fucosamine (FucNAc), the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacillformis ATCC BAA-1200 (NbPglL). In further embodiments, the PglL Glycan Substrate has a S-2 to S-1 reducing end structure of N-acetyl-altruronic acid-α1,3-4-amino-N-acetyl-fucosamine, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacilliformis ATCC BAA-1200 (NbPglL).
   In certain embodiments, the PglL Glycan Substrate has a reducing end structure of N-acetyl-fucosamine (FucNAc), the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL). In further embodiments, the PglL Glycan Substrate has a reducing end structure of N-acetyl-altruronic acid-α1,3-4-amino-N-acetyl-fucosamine, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL).
   In a certain embodiment, the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a certain embodiment, the PglL Glycan Substrate has a reducing end structure of N-acetyl-fucosamine (FucNAc) and the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a certain embodiment, the PglL Glycan Substrate has a reducing end structure of N-acetyl-altruronic acid-α1,3-4-amino-N-acetyl-fucosamine and the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a further embodiment, the PglL Glycan Substrate is S. sonnei O-antigen. In a further embodiment, the PglL Otase is NmPglL.
   In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 101, 103, 105, 108, 109, 111, 113, 117, 121, 125, 129, 131, 133, 199, 202, and 204. In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 101, 103, 105, 108, 109, 111, 113, 117, 121, 125, 129, 131, 133, 199, 202, and 204 and the PglL Otase is NmPglL. In a further embodiment, the PglL Glycan Substrate has a reducing end structure of N-acetyl-fucosamine (FucNAc). In a further embodiment, the PglL Glycan Substrate has a S-2 to S-1 reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 103, 105, 107, 109, and 111. In a certain embodiment, the modified carrier protein has the amino acid sequence of SEQ ID NO: 101 and the PglL Otase is NgPglL. In a further embodiment, the PglL Glycan Substrate has a reducing end structure of N-acetyl-fucosamine (FucNAc). In a further embodiment, the PglL Glycan Substrate has a S-2 to S-1 reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 103 and the PglL Otase is NlPglL. In a further embodiment, the PglL Glycan Substrate has a reducing end structure of N-acetyl-fucosamine (FucNAc). In a further embodiment, the PglL Glycan Substrate has a S-2 to S-1 reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 111 and the PglL Otase is NsPglL. In a further embodiment, the PglL Glycan Substrate has a reducing end structure of N-acetyl-fucosamine (FucNAc). In a further embodiment, the PglL Glycan Substrate has a S-2 to S-1 reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 113, 115, 117, 121, 123, 125, 127, 129, 131, and 133. In a certain embodiment, the modified carrier protein has the amino acid sequence of one of SEQ ID NOs: 101, 113, 115, 117, 121, 123, 125, 127, 129, 131, and 133 and the PglL Otase is NgPglL. In a further embodiment, the PglL Glycan Substrate has a reducing end structure of N-acetyl-fucosamine (FucNAc). In a further embodiment, the PglL Glycan Substrate has a S-2 to S-1 reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC23970PglL}), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a S-2 to S-1 reducing end structure of Glucuronic acid-β1,4-glucose. In a further embodiment, the PglL Glycan Substrate is an S. pneumoniae CPS having a reducing end structure of Glucose or a S-2 to S-1 reducing end structure of Glucuronic acid-β1,4-glucose. In a further embodiment, the PglL Glycan Substrate is S. pneumoniae sp.8 CPS.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a S-2 to S-1 reducing end structure of Rhamnose-β1,4-glucose. In a further embodiment, the PglL Glycan Substrate is an S. pneumoniae CPS having a reducing end structure of Glucose or a S-2 to S-1 reducing end structure of Rhamnose-β1,4-glucose. In a further embodiment, the PglL Glycan Substrate is S. pneumoniae sp.22A CPS.
   In certain embodiments, the modified carrier protein has the amino acid sequence of one of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 199, 202, and 204.
50. A composition, comprising:
   (a) a PglL Glycan Substrate,
   (b) a modified carrier protein as in any one of embodiments 1-36, and
   (c) a PglL OTase.
51. A conjugate comprising the modified carrier protein of any one of embodiments 1-36 and one or more other molecules.
52. The conjugate of embodiment 51, wherein the one or more other molecules are glycans and each is covalently attached to a serine or threonine residue of a GlycoTag.
53. The conjugate of embodiment 52, wherein the one or more glycans is endogenous to a Neisseria, Shigella, Salmonella, Streptococcus, Escherichia, Pseudomonas, Yersinia, Campylobacter, or Heliobacter cell.
   The conjugate of embodiment 51, wherein the modified carrier protein is coupled to a Shigella glycan [e.g. a Shigella sonnei glycan (such as S. sonnei O-antigen), or e.g. a Shigella flexneri glycan (such as Shigella flexneri 2a CPS), or a Shigella dysenteriae glycan] or coupled to a Streptococcus glycan [e.g. Streptococcus pneumoniae (such as Streptococcus pneumoniae sp. 12F CPS, S. pneumoniae sp. 8 CPS, S. pneumoniae sp. 14 CPS, S. pneumoniae sp. 23A CPS, S. pneumoniae sp. 33F CPS, or S. pneumoniae sp. 22A CPS)].
54. The conjugate of embodiment 52 or 53, wherein the one or more glycans each have a reducing end structure of Glucose, Galactose, Galactofuranose, Rhamnose, GlcNAc, GalNAc, FucNAc, DATDH, GATDH, HexNAc, deoxy HexNAc, diNAcBac, or Pse.
55. A composition comprising the conjugate of any one of embodiments 51-54.
   In certain embodiments, the PglL OTase is a Neisseria meningitidis PglL, Neisseria gonorrhoeae PglL, Neisseria lactamica 020-06 PglL, Neisseria lactamica ATCC 23970 PglL, Neisseria gonorrhoeae F62 PglL, Neisseria cinerea ATCC 14685 PglL, Neisseria mucosa PglL, Neisseria flavescens NRL30031/H210 PglL, Neisseria mucosa ATCC 25996 PglL, Neisseria sp. oral taxon 014 strain F0314 PglL, Neisseria arctica PglL, Neisseria shayeganii 871 PglL, Neisseria shayeganii 871 PglL, Neisseria sp. 83E34 PglL, Neisseria wadsworthii PglL, Neisseria elongata subsp. glycolytica ATCC 29315 PglL, Neisseria bacilliformis ATCC BAA-1200 PglL, Neisseria sp. oral taxon 020 str. F0370 PglL, Neisseria sp. 74A18 PglL, Neisseria weaver ATCC 51223 PglL, or Neisseria macacae ATCC 33926 PglL OTase.
   In certain embodiments, the glycan is endogenous to a Neisseria, Shigella, Salmonella, Streptococcus, Escherichia, Pseudomonas, Yersinia, Campylobacter, or Heliobacter cell.
   In certain embodiments, the PglL Glycan Substrate is an O-antigen. In certain embodiments, the PglL Glycan Substrate is S. sonnei O-antigen.
   In certain embodiments, the PglL Otase is a Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), and Neisseria bacillformis ATCC BAA-1200 (NbPglL), Neisseria mucosa ATCC 25996 (NmuPglL), or Neisseria shayeganii 871 (SEQ ID NO: 33, NsPglL) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC2397PglL}), or Neisseria gonorrhoeae F62 PglL (Ng_{F62PglL}) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase.
   In certain embodiments, the modified carrier protein has the amino acid sequence of one of SEQ ID NOs: 51, 53, and 55. In certain embodiments, the PglL Glycan Substrate has a reducing end structure of FucNAc and the modified carrier protein has the amino acid sequence SEQ ID NO: 51, 53, or 55. In certain embodiments, the PglL Glycan Substrate has a S-2 to S-1 reducing end structure of N-acetyl-altruronic acid-α1,3-4-amino-N-acetyl-fucosamine and the modified carrier protein has the amino acid sequence SEQ ID NO: 51, 53, or 55. In a further embodiment, the PglL Glycan Substrate is S. sonnei O-antigen. In a further embodiment, the PglL Otase is NmPglL.
   In certain embodiments, the modified carrier protein has the amino acid sequence of SEQ ID NO: 134 or 135. In certain embodiments, the PglL Glycan Substrate has a reducing end structure of GalNAc, FucNAc, or GlcNAc and the modified carrier protein has the amino acid sequence SEQ ID NO: 134 or 135. In certain embodiments, the PglL Glycan Substrate has a S-2 to S-1 reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine and the modified carrier protein has the amino acid sequence SEQ ID NO: 134 or 135. In a further embodiment, the PglL Glycan Substrate is a Shigella (e.g., S. sonnei or S. flexneri) or Streptococcus (e.g., S. pneumoniae) antigen having a S-2 to S-1 reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine and the modified carrier protein has the amino acid sequence SEQ ID NO: 134 or 135. In a further embodiment, the PglL Glycan Substrate is S. sonnei O-antigen, Shigella flexneri 2a CPS, or Streptococcus pneumoniae sp. 12F CPS. In a further embodiment, the PglL Otase is NmPglL.
   In a certain embodiment, the modified carrier protein has the amino acid sequence SEQ ID NO: 51, 199, 202, or 204. In a certain embodiment, the PglL Glycan Substrate has a reducing end structure of GlcNAc, GalNAc, FucNAc, or Glucose and the modified carrier protein has the amino acid sequence SEQ ID NO: 51, 199, 202, or 204. In a further embodiment, the PglL Glycan Substrate has a S-2 to S-1 reducing end structure of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine and the modified carrier protein has the amino acid sequence SEQ ID NO: 51, 199, 202, or 204. In further embodiments, the PglL Glycan Substrate is a Shigella sonnei glycan antigen (e.g. S. sonnei O-antigen), a Shigella flexneri glycan antigen (e.g. Shigella flexneri 2a CPS), a Shigella dysenteriae glycan antigen, or a Streptococcus pneumoniae glycan antigen (e.g. Streptococcus pneumoniae sp. 12F CPS, S. pneumoniae sp. 8 CPS, S. pneumoniae sp. 14 CPS, S. pneumoniae sp. 15A CPS, or S. pneumoniae sp. 33F CPS) having a S-2 to S-1 reducing end structure of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine and the modified carrier protein has the amino acid sequence SEQ ID NO: 51, 199, 202, or 204. In a further embodiment, the PglL Otase is NmPglL.
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacilliformis ATCC BAA-1200 (NbPglL). In certain embodiments, the PglL Glycan Substrate has a S-2 to S-1 reducing end structure of N-acetyl-altruronic acid-α1,3-4-amino-N-acetyl-fucosamine, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacilliformis ATCC BAA-1200 (NbPglL).
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL). In certain embodiments, the PglL Glycan Substrate has a S-2 to S-1 reducing end structure of N-acetyl-altruronic acid-α1,3-4-amino-N-acetyl-fucosamine, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL).
   In a certain embodiment, the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a certain embodiment, the PglL Glycan Substrate has a reducing end structure of FucNAc and the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a further embodiment, the PglL Glycan Substrate has a S-2 to S-1 reducing end of N-acetyl-altruronic acid-α1,3-4-amino-N-acetyl-fucosamine. In a further embodiment, the PglL Glycan Substrate is S. sonnei O-antigen. In a further embodiment, the PglL Otase is NmPglL.
   In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 100, 102, 105, 107, 109, 111, 113, 117, 121, 125, 129, 131, 133, 199, 202, and 204. In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 100, 102, 105, 107, 109, 111, 113, 117, 121, 125, 129, 131, 133, 199, 202, and 204 and the PglL Otase is NmPglL. In a further embodiment, the PglL Glycan Substrate has a reducing end structure of FucNAc. In a further embodiment, the PglL Glycan Substrate has a S-2 to S-1 reducing end of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 103, 105, 107, 109, and 111. In a certain embodiment, the modified carrier protein has the amino acid sequence of SEQ ID NO: 101 and the PglL Otase is NgPglL. In a further embodiment, the PglL Glycan Substrate has a reducing end structure of FucNAc. In a further embodiment, the PglL Glycan Substrate has a S-2 to S-1 reducing end of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 103 and the PglL Otase is NlPglL. In a further embodiment, the PglL Glycan Substrate has a reducing end structure of FucNAc. In a further embodiment, the PglL Glycan Substrate has a S-2 to S-1 reducing end of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 111 and the PglL Otase is NsPglL. In a further embodiment, the PglL Glycan Substrate has a reducing end structure of FucNAc. In a further embodiment, the PglL Glycan Substrate has a S-2 to S-1 reducing end of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 113, 115, 117, 121, 123, 125, 127, 129, 131, and 133. In a certain embodiment, the modified carrier protein has the amino acid sequence of one of SEQ ID NOs: 101, 113, 115, 117, 121, 123, 125, 127, 129, 131, and 133 and the PglL Otase is NgPglL. In a further embodiment, the PglL Glycan Substrate has a reducing end structure of FucNAc. In a further embodiment, the PglL Glycan Substrate has a S-2 to S-1 reducing end of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC23970PglL}), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a S-2 to S-1 reducing end of Glucuronic acid-β1,4-glucose. In a further embodiment, the PglL Glycan Substrate is a S. pneumoniae antigen with a S-2 to S-1 reducing end of Glucuronic acid-β1,4-glucose. In a further embodiment, the PglL Glycan Substrate is S. pneumoniae sp.8 CPS.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a S-2 to S-1 reducing end of Rhamnose-β1,4-glucose. In a further embodiment, the PglL Glycan Substrate is a S. pneumoniae antigen with a S-2 to S-1 reducing end of Rhamnose-β1,4-glucose. In a further embodiment, the PglL Glycan Substrate is S. pneumoniae sp.22A CPS.
   In certain embodiments, the modified carrier protein has the amino acid sequence of one of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 199, 202, and 204.
56. A method of producing an O-glycosylated modified carrier protein, comprising culturing a gram-negative bacterial host cell, wherein the gram-negative bacterial host cell:
   (a) produces a Lipid-Carrier-Linked PglL Glycan,
   (b) expresses a nucleotide sequence encoding a modified carrier protein as in any one of embodiments 1-36, operatively linked to a polynucleotide sequence encoding a periplasmic signal sequence, and
   (c) expresses a nucleotide sequence encoding a PglL OTase,
   thereby producing an O-glycosylated modified carrier protein.
   A method of producing an O-glycosylated modified carrier protein, comprising culturing a gram-negative bacterial host cell, wherein the gram-negative bacterial host cell:
      (a) expresses a nucleotide sequence encoding a PglL Glycan;
      (b) expresses one or more nucleotide sequence(s) encoding Glycosyltransferases capable of assembling a Lipid-Carrier-Linked PglL Glycan;
      (c) expresses a nucleotide sequence encoding a modified carrier protein as in any one of embodiments 1-36, operatively linked to a polynucleotide sequence encoding a periplasmic signal sequence, and
      (d) expresses a nucleotide sequence encoding a PglL OTase,
   thereby producing an O-glycosylated modified carrier protein.
57. The method of embodiment 56, wherein the PglL Glycan is essentially the same as a glycan endogenous to a Neisseria, Shigella, Salmonella, Streptococcus, Escherichia, Pseudomonas, Yersinia, Campylobacter, or Heliobacter cell.
58. The method of embodiment 56 or 57, wherein the PglL Glycan is characterized by having a Glucose, Galactose, Galactofuranose, Rhamnose, GlcNAc, GalNAc, FucNAc, DATDH, GATDH, HexNAc, deoxy HexNAc, diNAcBac, or Pse at the reducing end.
59. The method of embodiment 56, 57, or 58, wherein the PglL Glycan is endogenous to the host cell.
60. The method of embodiment 56, 57, 58, or 59, further comprising isolating the O-glycosylated modified carrier protein from the cell.
61. A composition comprising the O-glycosylated modified carrier protein produced by the method of one of embodiments 56-60.
   In certain embodiments, the PglL OTase is a Neisseria meningitidis PglL, Neisseria gonorrhoeae PglL, Neisseria lactamica 020-06 PglL, Neisseria lactamica ATCC 23970 PglL, Neisseria gonorrhoeae F62 PglL, Neisseria cinerea ATCC 14685 PglL, Neisseria mucosa PglL, Neisseria flavescens NRL30031/H210 PglL, Neisseria mucosa ATCC 25996 PglL, Neisseria sp. oral taxon 014 strain F0314 PglL, Neisseria arctica PglL, Neisseria shayeganii 871 PglL, Neisseria shayeganii 871 PglL, Neisseria sp. 83E34 PglL, Neisseria wadsworthii PglL, Neisseria elongata subsp. glycolytica ATCC 29315 PglL, Neisseria bacilliformis ATCC BAA-1200 PglL, Neisseria sp. oral taxon 020 str. F0370 PglL, Neisseria sp. 74A18 PglL, Neisseria weaver ATCC 51223 PglL, or Neisseria macacae ATCC 33926 PglL OTase.
   In certain embodiments, the Lipid-Carrier-Linked PglL Glycan is an O-antigen. In certain embodiments, the O-antigen is S. sonnei O-antigen.
   In certain embodiments, the PglL Otase is a Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), and Neisseria bacillformis ATCC BAA-1200 (NbPglL), Neisseria mucosa ATCC 25996 (NmuPglL), or Neisseria shayeganii 871 (SEQ ID NO: 33, NsPglL) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC2397PglL}), or Neisseria gonorrhoeae F62 PglL (Ng_{F62PglL}) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase.
   In certain embodiments, the modified carrier protein has the amino acid sequence of one of SEQ ID NOs: 51, 53, and 55. In certain embodiments, the PglL Glycan Substrate has a reducing end structure of FucNAc and the modified carrier protein has the amino acid sequence SEQ ID NO: 51, 53, or 55. In certain embodiments, the PglL Glycan Substrate has a S-2 to S-1 reducing end structure of N-acetyl-altruronic acid-α1,3-4-amino-N-acetyl-fucosamine and the modified carrier protein has the amino acid sequence SEQ ID NO: 51, 53, or 55. In a further embodiment, the PglL Glycan Substrate is S. sonnei O-antigen. In a further embodiment, the PglL Otase is NmPglL.
   In certain embodiments, the modified carrier protein has the amino acid sequence of SEQ ID NO: 134 or 135. In certain embodiments, the PglL Glycan Substrate has a reducing end structure of GalNAc, FucNAc, or GlcNAc and the modified carrier protein has the amino acid sequence SEQ ID NO: 134 or 135. In certain embodiments, the PglL Glycan Substrate has a reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine. In a further embodiment, the PglL Glycan Substrate is a Shigella (e.g., S. sonnei or Shigella flexneri) or Streptococcus (e.g., S. pneumoniae) antigen with a reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine. In a further embodiment, the PglL Glycan Substrate is S. sonnei O-antigen, Shigella flexneri 2a CPS, or Streptococcus pneumoniae sp. 12F CPS. In a further embodiment, the PglL Otase is NmPglL.
   In a certain embodiment, the modified carrier protein has the amino acid sequence SEQ ID NO: 51, 199, 202, or 204. In a certain embodiment, the PglL Glycan Substrate has a reducing end structure of GlcNAc, GalNAc, FucNAc, or Glucose and the modified carrier protein has the amino acid sequence SEQ ID NO: 51, 199, 202, or 204. In a certain embodiment, the PglL Glycan Substrate has a reducing end structure of Galactose-β1,4-glucose; Glucuronic acid-β1,4-glucose; N-acetyl-fucosamine-α1,3-N-acetyl-galactosamine; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine; and the modified carrier protein has the amino acid sequence SEQ ID NO: 51, 199, 202, or 204. In further embodiments, the PglL Glycan Substrate is a Shigella sonnei glycan antigen (e.g. S. sonnei O-antigen), a Shigella flexneri glycan antigen (e.g. Shigella flexneri 2a CPS), a Shigella dysenteriae glycan antigen, a Streptococcus pneumoniae glycan antigen (e.g. Streptococcus pneumoniae sp. 12F CPS, S. pneumoniae sp. 8 CPS, S. pneumoniae sp. 14 CPS, S. pneumoniae sp. 15A CPS, or S. pneumoniae sp. 33F CPS) with a reducing end structure of Galactose-β1,4-glucose; Glucuronic acid-β1,4-glucose; N-acetyl-fucosamine-α1,3-N-acetyl-galactosamine; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine; and the modified carrier protein has the amino acid sequence SEQ ID NO: 51, 199, 202, or 204. In a further embodiment, the PglL Otase is NmPglL.
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacilliformis ATCC BAA-1200 (NbPglL). In certain embodiments, the PglL Glycan Substrate has a reducing end structure of N-acetyl-altruronic acid-α1,3-4-amino-N-acetyl-fucosamine, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacilliformis ATCC BAA-1200 (NbPglL).
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL). In certain embodiments, the PglL Glycan Substrate has a reducing end structure of N-acetyl-altruronic acid-α1,3-4-amino-N-acetyl-fucosamine, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL).
   In a certain embodiment, the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a certain embodiment, the PglL Glycan Substrate has a reducing end structure of FucNAc and the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a certain embodiment, the PglL Glycan Substrate has a reducing end structure of N-acetyl-altruronic acid-α1,3-4-amino-N-acetyl-fucosamine and the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a further embodiment, the PglL Glycan Substrate is S. sonnei O-antigen. In a further embodiment, the PglL Otase is NmPglL.
   In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 100, 102, 105, 107, 109, 111, 113, 117, 121, 125, 129, 131, 133, 199, 202, and 204. In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 100, 102, 105, 107, 109, 111, 113, 117, 121, 125, 129, 131, 133, 199, 202, and 204 and the PglL Otase is NmPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 103, 105, 107, 109, and 111. In a certain embodiment, the modified carrier protein has the amino acid sequence of SEQ ID NO: 101 and the PglL Otase is NgPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 103 and the PglL Otase is NlPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 111 and the PglL Otase is NsPglL. In a further embodiment, the PglL Glycan Substrate has a reducing end structure of FucNAc. In a further embodiment, the PglL Glycan Substrate has a S-2 to S-1 reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 113, 115, 117, 119, 121, 123, 125, 127, 129, 131, and 133. In a certain embodiment, the modified carrier protein has the amino acid sequence of one of SEQ ID NOs: 101, 113, 115, 117, 119, 121, 123, 125, 127, 129, 131, and 133 and the PglL Otase is NgPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC23970PglL}), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a reducing end structure of Glucuronic acid-β1,4-glucose. In a further embodiment, the PglL Glycan Substrate is a Streptococcus (e.g., S. pneumoniae) antigen with a reducing end structure of glucose (e.g., Glucuronic acid-β1,4-glucose). In a further embodiment, the PglL Glycan Substrate is S. pneumoniae sp.8 CPS.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a reducing end structure of Rhamnose-β1,4-glucose. In a further embodiment, the PglL Glycan Substrate is a Streptococcus (e.g., S. pneumoniae) antigen with a reducing end structure of Glucose (e.g., Rhamnose-β1,4-glucose). In a further embodiment, the PglL Glycan Substrate is S. pneumoniae sp.22A CPS.
   In certain embodiments, the modified carrier protein has the amino acid sequence of one of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 199, 202, and 204.
62. A method of producing an O-glycosylated modified carrier protein, comprising culturing a gram-negative bacterial host cell, wherein the gram-negative bacterial host cell:
   (a) comprises lipid-Carrier-Linked PglL Glycan Substrate,
   (b) comprises in the periplasm a modified carrier protein,
      the modified carrier protein being characterized by a carrier protein comprising at least one NgGlycoTag, NlGlycoTag, or NsGlycoTag, and
   (c) comprises a Neisseria PglL OTase.
63. The method of embodiment 62, wherein the Lipid-Carrier-Linked PglL Glycan Substrate comprises at the reducing end a Glucose, Galactose, Galactofuranose, Rhamnose, GlcNAc, GalNAc, FucNAc, DATDH, GATDH, HexNAc, deoxy HexNAc, diNAcBac, or Pse.
64. The method of embodiment 62 or 63, wherein the Lipid-Carrier-Linked PglL Glycan Substrate is endogenous to the host cell.
65. The method of one of embodiments 62-64, further comprising isolating an O-glycosylated modified carrier protein from the cell.
   In certain embodiments, the method comprises isolated an O-glycosylated modified carrier protein from the periplasm of the cell.
   In certain embodiments, the PglL OTase is a Neisseria meningitidis PglL, Neisseria gonorrhoeae PglL, Neisseria lactamica 020-06 PglL, Neisseria lactamica ATCC 23970 PglL, Neisseria gonorrhoeae F62 PglL, Neisseria cinerea ATCC 14685 PglL, Neisseria mucosa PglL, Neisseria flavescens NRL30031/H210 PglL, Neisseria mucosa ATCC 25996 PglL, Neisseria sp. oral taxon 014 strain F0314 PglL, Neisseria arctica PglL, Neisseria shayeganii 871 PglL, Neisseria shayeganii 871 PglL, Neisseria sp. 83E34 PglL, Neisseria wadsworthii PglL, Neisseria elongata subsp. glycolytica ATCC 29315 PglL, Neisseria bacilliformis ATCC BAA-1200 PglL, Neisseria sp. oral taxon 020 str. F0370 PglL, Neisseria sp. 74A18 PglL, Neisseria weaver ATCC 51223 PglL, or Neisseria macacae ATCC 33926 PglL OTase.
   In certain embodiments, the glycan is an O-antigen. In certain embodiments, the O-antigen is S. sonnei O-antigen.
   In certain embodiments, the PglL Otase is a Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), and Neisseria bacilliformis ATCC BAA-1200 (NbPglL), Neisseria mucosa ATCC 25996 (NmuPglL), or Neisseria shayeganii 871 (SEQ ID NO: 33, NsPglL) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC23970PglL}), or Neisseria gonorrhoeae F62 PglL (Ng_{F62PglL}) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase.
   In certain embodiments, the modified carrier protein has the amino acid sequence of one of SEQ ID NOs: 51, 53, and 55. In certain embodiments, the PglL Glycan Substrate has a reducing end structure of FucNAc and the modified carrier protein has the amino acid sequence SEQ ID NO: 51, 53, or 55. In certain embodiments, the PglL Glycan Substrate has a S-2 to S-1 reducing end structure of N-acetyl-altruronic acid-α1,3-4-amino-N-acetyl-fucosamine. In a further embodiment, the PglL Glycan Substrate is S. sonnei O-antigen. In a further embodiment, the PglL Otase is NmPglL.
   In certain embodiments, the modified carrier protein has the amino acid sequence of SEQ ID NO: 134 or 135. In certain embodiments, the PglL Glycan Substrate has a reducing end structure of GalNAc, FucNAc, or GlcNAc and the modified carrier protein has the amino acid sequence SEQ ID NO: 134 or 135. In certain embodiments, the PglL Glycan Substrate has a reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine. In a further embodiment, the PglL Glycan Substrate is a Shigella or Streptococcus antigen with a reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine. In a further embodiment, the PglL Glycan Substrate is S. sonnei O-antigen, Shigella flexneri 2a CPS, or Streptococcus pneumoniae sp. 12F CPS. In a further embodiment, the PglL Otase is NmPglL.
   In a certain embodiment, the modified carrier protein has the amino acid sequence SEQ ID NO: 51, 199, 202, or 204. In a certain embodiment, the PglL Glycan Substrate has a reducing end structure of GlcNAc, GalNAc, FucNAc, or Glucose and the modified carrier protein has the amino acid sequence SEQ ID NO: 51, 199, 202, or 204. In a certain embodiment, the PglL Glycan Substrate has a S-2 to S-1 reducing end structure of Galactose-β1,4-glucose; Glucuronic acid-β1,4-glucose; N-acetyl-fucosamine-α1,3-N-acetyl-galactosamine; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine; and the modified carrier protein has the amino acid sequence SEQ ID NO: 51, 199, 202, or 204. In further embodiments, the PglL Glycan Substrate is a Shigella or Streptococcus antigen with a a S-2 to S-1 reducing end structure of Galactose-β1,4-glucose; Glucuronic acid-β1,4-glucose; N-acetyl-fucosamine-α1,3-N-acetyl-galactosamine; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine; and the modified carrier protein has the amino acid sequence SEQ ID NO: 51, 199, 202, or 204. In further embodiments, the PglL Glycan Substrate is a Shigella sonnei glycan antigen (e.g. S. sonnei O-antigen), a Shigella flexneri glycan antigen (e.g. Shigella flexneri 2a CPS), a Shigella dysenteriae glycan antigen, a Streptococcus pneumoniae glycan antigen (e.g. Streptococcus pneumoniae sp. 12F CPS, S. pneumoniae sp. 8 CPS, S. pneumoniae sp. 14 CPS, S. pneumoniae sp. 15A CPS, or S. pneumoniae sp. 33F CPS). In a further embodiment, the PglL Otase is NmPglL.
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacilliformis ATCC BAA-1200 (NbPglL). In a further embodiment, the PglL Glycan Substrate has an S-2 to S-1 reducing end structure of N-acetyl-altruronic acid-α1,3-4-amino-N-acetyl-fucosamine.
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL). In a further embodiment, the PglL Glycan Substrate has an S-2 to S-1 reducing end structure of N-acetyl-altruronic acid-α1,3-4-amino-N-acetyl-fucosamine.
   In a certain embodiment, the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a certain embodiment, the PglL Glycan Substrate has a reducing end structure of FucNAc and the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a further embodiment, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen. In a further embodiment, the PglL Otase is NmPglL.
   In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 101, 103, 105, 107, 109, 111, 113, 117, 121, 125, 129, 131, 133, 199, 202, and 204. In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 101, 103, 105, 107, 109, 111, 113, 117, 121, 125, 129, 131, 133, 199, 202, and 204 and the PglL Otase is NmPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 103, 105, 107, 109, and 111. In a certain embodiment, the modified carrier protein has the amino acid sequence of SEQ ID NO: 101 and the PglL Otase is NgPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 103 and the PglL Otase is NlPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 111 and the PglL Otase is NsPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 113, 115, 117, 121, 123, 125, 127, 129, 131, and 133. In a certain embodiment, the modified carrier protein has the amino acid sequence of one of SEQ ID NOs: 101, 113, 115, 117, 121, 123, 125, 127, 129, 131, and 133 and the PglL Otase is NgPglL. In a further embodiment, the PglL Glycan Substrate has a reducing end structure of FucNAc. In a further embodiment, the PglL Glycan Substrate has a reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC23970PglL}), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a reducing end structure of Glucuronic acid-β1,4-glucose. In a further embodiment, the PglL Glycan Substrate is a Streptococcus antigen with a reducing end structure of Glucose (e.g., Glucuronic acid-β1,4-glucose). In a further embodiment, the PglL Glycan Substrate is S. pneumoniae sp.8 CPS.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase. In a further embodiment, the PglL Glycan Substrate has a reducing end structure of Glucose. In a further embodiment, the PglL Glycan Substrate has a reducing end structure of Rhamnose-β1,4-glucose. In a further embodiment, the PglL Glycan Substrate is a Streptococcus antigen with a reducing end structure of Glucose (e.g., Rhamnose-β1,4-glucose). In a further embodiment, the PglL Glycan Substrate is S. pneumoniae sp.22A CPS.
   In certain embodiments, the modified carrier protein has the amino acid sequence of one of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 199, 202, and 204.
66. A method of making a conjugate, comprising contacting a PglL OTase and a PglL Glycan Substrate in the presence of a modified carrier protein as in any one of embodiments 1 to 36; thereby making the conjugate, optionally then isolating the conjugate.
67. The method of embodiment 66 wherein the conjugate is a bioconjugate and contacting occurs in the periplasm of a gram-negative bacterial cell.
68. A composition comprising a O-glycosylated modified carrier protein produced by the method of embodiment 66 or 67.
   In certain embodiments, the modified carrier protein is characterized by a carrier protein selected from the group consisting of cholera toxin b subunit (CTB), tetanus toxoid (TT), tetanus toxin C fragment (TTc), diphtheria toxoid (DT), CRM197, Pseudomonas aeruginosa exotoxin A (EPA), C. jejuni Acriflavine resistance protein A (CjAcrA), E. coli Acriflavine resistance protein A (EcAcrA), and Pseudomonas aeruginosa PcrV (PcrV) comprising at least one GlycoTag.
   In certain embodiments, the PglL Glycan Substrate is endogenous to a Neisseria, Shigella, Salmonella, Streptococcus, Escherichia, Pseudomonas, Yersinia, Campylobacter, or Heliobacter cell.
   In certain embodiments, the PglL Glycan Substrate is an O-antigen. In certain embodiments, the O-antigen is S. sonnei O-antigen.
   In certain embodiments, the PglL Glycan Substrate has a reducing end structure of Glucose, Galactose, Galactofuranose, Rhamnose, GlcNAc, GalNAc, FucNAc, DATDH, GATDH, HexNAc, deoxy HexNAc, diNAcBac, or Pse.
   In certain embodiments, the PglL OTase is a Neisseria meningitidis PglL, Neisseria gonorrhoeae PglL, Neisseria lactamica 020-06 PglL, Neisseria lactamica ATCC 23970 PglL, Neisseria gonorrhoeae F62 PglL, Neisseria cinerea ATCC 14685 PglL, Neisseria mucosa PglL, Neisseria flavescens NRL30031/H210 PglL, Neisseria mucosa ATCC 25996 PglL, Neisseria sp. oral taxon 014 strain F0314 PglL, Neisseria arctica PglL, Neisseria shayeganii 871 PglL, Neisseria shayeganii 871 PglL, Neisseria sp. 83E34 PglL, Neisseria wadsworthii PglL, Neisseria elongata subsp. glycolytica ATCC 29315 PglL, Neisseria bacilliformis ATCC BAA-1200 PglL, Neisseria sp. oral taxon 020 str. F0370 PglL, Neisseria sp. 74A18 PglL, Neisseria weaver ATCC 51223 PglL, or Neisseria macacae ATCC 33926 PglL OTase.
   In certain embodiments, the PglL Otase is a Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), and Neisseria bacillformis ATCC BAA-1200 (NbPglL), Neisseria mucosa ATCC 25996 (NmuPglL), or Neisseria shayeganii 871 (SEQ ID NO: 33, NsPglL) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC23970PglL}), or Neisseria gonorrhoeae F62 PglL (Ng_{F62PglL}) PglL Otase.
   In certain embodiments, the PglL Otase is a Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase.
   In certain embodiments, the modified carrier protein has the amino acid sequence of one of SEQ ID NOs: 51, 53, and 55. In certain embodiments, the PglL Glycan Substrate has a reducing end structure of FucNAc and the modified carrier protein has the amino acid sequence SEQ ID NO: 51, 53, or 55. In a further embodiment, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen. In a further embodiment, the PglL Otase is NmPglL.
   In certain embodiments, the modified carrier protein has the amino acid sequence of SEQ ID NO: 134 or 135. In certain embodiments, the PglL Glycan Substrate has a reducing end structure of GalNAc, FucNAc, or GlcNAc and the modified carrier protein has the amino acid sequence SEQ ID NO: 134 or 135. In certain embodiments, the PglL Glycan Substrate has a reducing end structure of N-acetyl-fucosamine-α1,3-N-acetyl-galactosamine; N-acetyl-altruronic acid-α1,3-4-amino-N-acetyl-fucosamine; or Rhamnose-β1,4-N-acetylgalactosamine. In a further embodiment, the PglL Glycan Substrate is a Shigella or Streptococcus antigen. In a further embodiment, the PglL Glycan Substrate is S. sonnei O-antigen, Shigella flexneri 2a CPS, or Streptococcus pneumoniae sp. 12F CPS. In a further embodiment, the PglL Otase is NmPglL.
   In a certain embodiment, the modified carrier protein has the amino acid sequence SEQ ID NO: 51, 199, 202, or 204. In a certain embodiment, the PglL Glycan Substrate has a reducing end structure of GlcNAc, GalNAc, FucNAc, or Glucose and the modified carrier protein has the amino acid sequence SEQ ID NO: 51, 199, 202, or 204. In further embodiments, the PglL Glycan Substrate has an S-2 to S-1 reducing end structure of Galactose-β1,4-glucose; Glucuronic acid-β1,4-glucose; N-acetyl-fucosamine-α1,3-N-acetyl-galactosamine; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine. In further embodiments, the PglL Glycan Substrate is a Shigella sonnei glycan antigen e.g. S. sonnei O-antigen, a Shigella flexneri glycan antigen e.g. Shigella flexneri 2a CPS, a Shigella dysenteriae glycan antigen, a Streptococcus pneumoniae glycan antigen e.g. Streptococcus pneumoniae sp. 12F CPS, S. pneumoniae sp. 8 CPS, S. pneumoniae sp. 14 CPS, S. pneumoniae sp. 15A CPS, or S. pneumoniae sp. 33F CPS. In a further embodiment, the PglL Otase is NmPglL.
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), or Neisseria bacilliformis ATCC BAA-1200 (NbPglL). In further embodiments, the PglL Glycan Substrate has an S-2 to S-1 reducing end structure of N-acetyl-altruronic acid-α1,3-4-amino-N-acetyl-fucosamine.
   In certain embodiments, the PglL Glycan Substrate has a reducing end structure of FucNAc, the modified carrier protein has the amino acid sequence SEQ ID NO: 51, and the PglL Otase is Neisseria meningitidis PglL (NmPglL), Neisseria gonorrhoeae PglL (NgPglL), Neisseria lactamica 020-06 (NlPglL), Neisseria elongate subsp. glycolytica ATCC 29315 (NePglL), Neisseria bacilliformis ATCC BAA-1200 (NbPglL), Neisseria mucosa ATCC 25996 (NmuPglL), or Neisseria shayeganii 871 (SEQ ID NO: 33, NsPglL). In further embodiments, the PglL Glycan Substrate has an S-2 to S-1 reducing end structure of N-acetyl-altruronic acid-α1,3-4-amino-N-acetyl-fucosamine.
   In a certain embodiment, the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In a certain embodiment, the PglL Glycan Substrate has a reducing end structure of FucNAc and the modified carrier protein has the amino acid sequence of one of SEQ ID NOs: 57, 59, 61, 63, 65, 67, 69, 71, 73, 75, 77, 79, 81, 83, 85, 87, 89, 91, 93, 95, 97, and 99. In further embodiments, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen. In a further embodiment, the PglL Otase is NmPglL.
   In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 101, 103, 105, 107, 109, 111, 113, 117, 121, 125, 129, 131, 133, 199, 202, and 204. In a certain embodiment, the modified carrier protein has the amino acid sequence of one of SEQ ID NOs: 51, 57, 59, 61, 63, 65, 67, 69, 71, 73, 75, 77, 79, 81, 83, 85, 87, 89, 91, 93, 95, 97, 99, 101, 103, 105, 107, 109, 111, 113, 117, 121, 125, 129, 131, 133, 199, 202, and 204 and the PglL Otase is NmPglL. In a further embodiment, the PglL Glycan Substrate has a reducing end structure of FucNAc. In further embodiments, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 103, 105, 107, 109, and 111. In a certain embodiment, the modified carrier protein has the amino acid sequence of SEQ ID NO: 101 and the PglL Otase is NgPglL. In a further embodiment, the PglL Glycan Substrate has a reducing end structure of FucNAc. In further embodiments, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 103 and the PglL Otase is NlPglL. In a further embodiment, the PglL Glycan Substrate has a reducing end structure of FucNAc. In further embodiments, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen. In a certain embodiment, the modified carrier protein has the amino acid sequence of SEQ ID NO: 111 and the PglL Otase is NsPglL. In a further embodiment, the PglL Glycan Substrate has a reducing end structure of FucNAc. In further embodiments, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence of one of SEQ ID NOs: 101, 113, 115, 117, 121, 123, 125, 127, 129, 131, and 133. In a certain embodiment, the modified carrier protein has the amino acid sequence of one of SEQ ID NOs: 101, 113, 115, 117, 121, 123, 125, 127, 129, 131, and 133 and the PglL Otase is NgPglL. In a further embodiment, the PglL Glycan Substrate has a reducing end structure of FucNAc. In further embodiments, the PglL Glycan Substrate has an S-2 to S-1 reducing end structure of N-acetyl-altruronic acid-α1,3-4-amino-N-acetyl-fucosamine. In a further embodiment, the PglL Glycan Substrate is S. sonnei O-antigen.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), Neisseria lactamica ATCC 23970 PglL (Nl_{ATCC23970PglL}), or Neisseria gonorrhoeae F62 PglL (Ng_{F62PglL}) PglL Otase. In a further embodiment, the PglL Glycan Substrate has a reducing end structure of Glucose. In further embodiments, the PglL Glycan Substrate has an S-2 to S-1 reducing end structure of Glucuronic acid-β1,4-glucose. In a further embodiment, the PglL Glycan Substrate is a Streptococcus antigen with a reducing end structure of Glucose (e.g., an S-2 to S-1 reducing end structure of Glucuronic acid-β1,4-glucose). In a further embodiment, the PglL Glycan Substrate is S. pneumoniae sp.8 CPS.
   In a certain embodiment, the modified carrier protein has the amino acid sequence SEQ ID NO: 51 and the PglL Otase is Neisseria meningitidis PglL (NmPglL), Neisseria gonorrhoeae PglL (NgPglL), Neisseria lactamica 020-06 PglL (NlPglL), or Neisseria gonorrhoeae F62 PglL (Ng_{F62PglL}) PglL Otase. In a further embodiment, the PglL Glycan Substrate has a reducing end structure of Glucose. In further embodiments, the PglL Glycan Substrate has an S-2 to S-1 reducing end structure of Rhamnose-β1,4-glucose. In a further embodiment, the PglL Glycan Substrate is a Streptococcus antigen with a reducing end structure of Glucose (e.g., an S-2 to S-1 reducing end structure of Rhamnose-β1,4-glucose). In a further embodiment, the PglL Glycan Substrate is S. pneumoniae sp.22A CPS.
   In certain embodiments, the modified carrier protein has the amino acid sequence of one of SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 199, 202, and 204.
69. An immunogenic composition comprising the modified carrier protein as in any one of embodiments 1 to 36 covalently attached to one or more immunogenic glycans.
   An immunogenic composition comprising the modified carrier protein as in any one of embodiments 1 to 36 covalently attached to one or more immunogenic PglL Glycan Substrates.
   In certain embodiments, the PglL Glycan Substrate is endogenous to a Neisseria, Shigella, Salmonella, Streptococcus, Escherichia, Pseudomonas, Yersinia, Campylobacter, or Heliobacter cell.
   In certain embodiments, the PglL Glycan Substrate is an O-antigen. In certain embodiments, the O-antigen is S. sonnei O-antigen.
   In certain embodiments, the PglL Glycan Substrate has a reducing end structure of Glucose, Galactose, Galactofuranose, Rhamnose, GlcNAc, GalNAc, FucNAc, DATDH, GATDH, HexNAc, deoxy HexNAc, diNAcBac, or Pse. In further embodiments, the lipid-linked PglL Glycan Substrate has a reducing end structure of DATDH, GlcNAc, GalNAc, FucNAc, Galactose, or Glucose. In further embodiments, the lipid-linked PglL Glycan Substrate has a reducing end structure of GlcNAc, GalNAc, FucNAc, or Glucose. In further embodiments, the PglL Glycan Substrate has a S-2 to S-1 reducing end structure of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine. In further embodiments, the PglL Glycan Substrate is a Shigella (e.g., S. sonnei or Shigella flexneri) or Streptococcus (e.g., S. pneumoniae) antigen. In further embodiments, the PglL Glycan Substrate is S. sonnei O-antigen, Shigella flexneri 2a CPS, Streptococcus pneumoniae sp. 12F CPS, S. pneumoniae sp. 8 CPS, S. pneumoniae sp. 14 CPS, S. pneumoniae sp. 15A CPS, S. pneumoniae sp. 33F CPS, S. pneumoniae sp. 22A CPS, or S. flexneri sp. 2a CPS.
70. A method of inducing an antibody response in a mammal, comprising administering to the mammal an immunologically effective amount of the immunogenic composition of embodiment 69.
71. The immunogenic composition as in embodiment 69 for use in inducing an antibody response in a mammal.
   The immunogenic composition as in embodiment 69 for use in inducing an immune response in a mammal.
72. Use of the immunogenic composition of embodiment 69 for inducing an antibody response in a mammal.
   Use of the immunogenic composition of embodiment 69 for inducing an immune response in a mammal.
73. Use of the immunogenic composition of embodiment 69 for the manufacture of a medicament for inducing an antibody response in a mammal.
   Use of the immunogenic composition of embodiment 69 for the manufacture of a medicament for inducing an immune response in a mammal.
74. The immunogenic composition as in embodiment 69 for use in the treatment or prevention of a disease caused by Streptococcus pneumoniae infection.
   The immunogenic composition of embodiment 74, wherein the disease caused by Streptococcus pneumoniae infection is pneumonia, invasive pneumococcal disease (IPD), exacerbations of chronic obstructive pulmonary disease (COPD), otitis media, meningitis, bacteraemia, pneumonia and/or conjunctivitis.
   The immunogenic composition as in embodiment 69 for use in inducing an immune response against a Streptococcus pneumoniae glycan in a mammal.
75. Use of the immunogenic composition of embodiment 69 for inducing an antibody response against a Streptococcus pneumoniae glycan in a mammal.
   Use of the immunogenic composition of embodiment 69 for inducing an immune response against a Streptococcus pneumoniae glycan in a mammal.
76. Use of the immunogenic composition of embodiment 69 for the manufacture of a medicament for inducing an antibody response against a Streptococcus pneumoniae glycan in a mammal.
   Use of the immunogenic composition of embodiment 69 for the manufacture of a medicament for inducing an immune response against a Streptococcus pneumoniae glycan in a mammal.

### BRIEF DESCRIPTION OF THE DRAWINGS/FIGURES

FIG. 1—overview of a Neisseria O-linked, oligosaccharyltransferase-mediated glycosylation pathway. Adapted from FIG. 3(b) of [2].

FIG. 2—Western blot (FIG. 2A) and Coomassie blue (FIG. 2B) results from assaying NmPglL transfer of S. sonnei O-antigen to rEPA1 (columns #1 and #4), to rEPA2 (column #2), and rEPA3 (column #3). Antibodies against His-tagged EPA were used.

FIG. 3—The stability of the rEPA1-S. sonnei O-antigen bioconjugate was studied at three different temperatures (−80° C., 2-8° C., and room temperature (RT) 20-25° C.) for a time of six months. Additionally, five freeze/thaw cycles (5 FT) on purified rEPA1-S. sonnei O-antigen were performed. FIG. 3 depicts the SEC-HPLC readouts of samples taken at zero months, two weeks, one month, three months, and six months.

FIG. 4—Western blot results (using antibodies against S. sonnei O-antigen and EPA) probing bleed sera taken at zero, twenty-one, and twenty-eight days from New Zealand White Rabbits which were subcutaneously injected at zero, seven, ten, and eighteen days with an rEPA1-S. sonnei O-antigen bioconjugate composition comprising (FIG. 4A) 2 μg of sugar, 40 μg of protein, and non-Freund's adjuvant or (FIG. 4B) 10 μg of sugar, 200 μg of protein, and non-Freund's adjuvant.

FIG. 5—Western blot results from assaying NmPglL transfer of S. sonnei O-antigen, S. flexneri 2a O-antigen, and Streptococcus pneumoniae 12F CPS onto rEPA1 or rEPA43. Anti-His antibodies towards His-tagged EPA were used.

FIG. 6—Western blot results from assaying NmPglL transfer of S. sonnei O-antigen onto mAcrA, mPcrV, mCrm197 (column “3.1”), or m2Crm197 (column “3.2”). Anti-His antibodies towards His-tagged EPA were used.

FIG. 7—Depiction of surface-exposed Pseudomonas exotoxin A (EPA) residues modified to produce rEPA4-rEPA23 (FIG. 7A) and rEPA24-rEPA25 (FIG. 7B). Residues numbered with respect to SEQ ID NO: 1, structure adapted from Protein Data Bank (PDB) ID 1IKQ.

FIG. 8—Western blot results from assaying NmPglL transfer of lipid-carrier-linked S. sonnei O-antigen to rEPA4-rEPA15, rEPA24-rEPA25 (FIG. 8A) and rEPA16-rEPA25 (FIG. 8B) in vivo. Antibodies against the histidine tag (top gels) and against S. sonnei O-antigen (bottom gels) were used.

FIG. 9—Western blot results from assaying whether NgPglL, NlPglL, NePglL, NbPglL, and NmuPglL (FIG. 9A) as well as NsPglL (FIG. 9B) transfer lipid-carrier-linked S. sonnei O-antigen to carrier proteins containing an endogenous GlycoTag (i.e., to carrier protein rEPA26-rEPA31, respectively). FIG. 9B also depicts the Western blot results of having assayed NmPglL transfer of S. sonnei O-antigen onto rEPA26-rEPA31. Antibodies against the histidine tag (top gels) and against S. sonnei O-antigen (bottom gels) were used.

FIG. 10—Western blot results from assaying NgPglL transfer of lipid-carrier-linked S. sonnei O-antigen to rEPA332-rEPA39. “N-terminal” was EPA with NgPilin GlycoTag sequence SEQ ID NO: 145 operably linked to its N-terminus. “N/C-terminal” was EPA with two copies of the NgPilin GlycoTag sequence SEQ ID NO: 145, one at its N-terminus and a second at its C-terminus. Antibodies against EPA (FIG. 10A) and against S. sonnei O-antigen (FIG. 10B) were used.

FIG. 11—Western blot and Coommassie blue staining results from assaying NmPglL transfer of lipid-carrier-linked S. sonnei O-antigen to rEPA32, rEPA34, rEPA36, rEPA38, rEPA40, rEPA41, and rEPA42 in vivo. Antibodies against EPA were used.

FIG. 12—Western blot and Coomassie blue staining results from assaying NmPglL, NgPglL (“N. gonorrhoeae 1”), NlPglL (“N. lactamica 1”), Nl_{ATCC23970PglL} (“N. lactamica 2”), and Ng_{F62}PglL (“N. gonorrhoeae 2”) transfer of Pneomococcal Sp. 8 CPS glycan onto rEPA1 (FIG. 12A). Protein Kinase (“PK”) treatment of samples abolished NmPglL, NgPglL (“N. gonorrhoeae 1”), NlPglL (“N. lactamica 1”), and Ng_{F62}PglL (“N. gonorrhoeae 2”) signals, indicating that the Pneomococcal Sp. 8 CPS glycan was linked to the rEPA protein (FIG. 12B). Antibodies against Pneomococcal Sp. 8 CPS glycan were used.

FIG. 13—Western blot and Coomassie blue staining results from assaying NmPglL, NgPglL (“N. gonorrhoeae 1”), NlPglL (“N. lactamica 1”), and Ng_{F62}PglL (“N. gonorrhoeae 2”) transfer of Pneomococcal Sp. 22A CPS glycan onto rEPA1. Protein Kinase (“+PK”) treatment of samples abolished NmPglL, NgPglL (“N. gonorrhoeae 1”), NlPglL (“N. lactamica 1”), and Ng_{F62}PglL (“N. gonorrhoeae 2”) signals, indicating that the Pneomococcal Sp. 22A CPS glycan was linked to the rEPA1 protein. Antibodies against Pneomococcal Sp. 22A CPS glycan were used.

### DETAILED DESCRIPTION OF THE INVENTION

The present invention provides modified carrier proteins incorporating one or more GlycoTag and their use for in vivo or in vitro bioconjugation.

### Definitions

To facilitate an understanding of the present invention, a number of terms and phrases are defined below. Alternate forms (tenses) of these terms and phrases are also encompassed herein. Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

“Comprise” (“comprising” or “comprises”) as used herein is open-ended and means “including, but not limited to.” “Having” is used herein as a synonym of comprising. It is understood that wherever embodiments are described herein with the language “comprising,” such embodiments encompass those described in terms of “consisting of” and/or “consisting essentially of”. “Comprises therein” or “comprising therein” means that the referenced molecule, amino acid sequence, or nucleotide sequence has incorporated within it a GlycoTag molecule, amino acid sequence or nucleotide sequence, respectively. With respect to, for example, a “carrier protein comprising therein a GlycoTag,” the nucleotide sequence encoding that carrier protein has, between the 5′ and 3′ ends, a nucleotide sequence encoding a GlycoTag, likewise the carrier protein amino acid sequence has, between the N- and C-terminus, a GlycoTag amino acid sequence.

As used in the description and the appended claims, the singular forms “a,” “an,” and “the” include plural referents unless the context clearly dictates otherwise. Thus, for example, reference to “a GlycoTag” encompasses one or more GlycoTags.

“About” or “approximately” mean roughly, around, or in the regions of. The terms “about” or “approximately” further mean within an acceptable contextual error range for the particular value as determined by one of ordinary skill in the art, which will depend in part on how the value is measured, i.e. the limitations of the measurement system or the degree of precision required for a particular purpose. When the terms “about” or “approximately” are used in conjunction with a numerical range, it modifies that range by extending the boundaries above and below the numerical values set forth. For example “between about 5.5 to 6.5 g/l” means the boundaries of the numerical range extend below 5.5 and above 6.5 so that the particular value in question achieves the same functional result as within the range. For example, “about” and “approximately” can mean within 1 or more than 1 standard deviation as per the practice in the art. Alternatively, “about” and “approximately” can mean a range of up to 20%, up to 10%, up to 5%, or up to 1% of a given value.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated, a process comprising a step of mixing two or more components does not require any specific order of mixing. Components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not itself mean that the steps must be performed in that order (i.e., step 1 then step 2 then step 3, etc.). In certain embodiments, the word “then” is used to specify the order of a method's steps.

“Essentially the same” herein means a high degree of similarity between at least two molecules (including structure or function) or numeric values such that one of skill in the art would consider the difference to be immaterial, negligible, and/or statistically insignificant. For example, a first polypeptide, conjugate, antibody, polynucleotide, vector, cell, composition, or molecule is “essentially the same” as a second polypeptide, conjugate, antibody, polynucleotide, vector, cell, composition, or molecule herein if the first has only immaterial differences in structure and function as compared to the second. “Essentially the same” herein encompasses “the same.”

An “effective amount” means an amount sufficient to cause the referenced effect or outcome. An “effective amount” can be determined empirically and in a routine manner using known techniques in relation to the stated purpose. In certain embodiments, a composition comprises an immunologically effective amount of an antigen, adjuvant, or both. In certain embodiments, an “effective amount” in the context of administering a therapy (e.g. an immunogenic composition or vaccine of the invention) to a subject refers to the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a bacterial infection or symptom associated therewith; (ii) reduce the duration of a bacterial infection or symptom associated therewith; (iii) prevent the progression of a bacterial infection or symptom associated therewith; (iv) cause regression of a bacterial infection or symptom associated therewith; (v) prevent the development or onset of a bacterial infection, or symptom associated therewith; (vi) prevent the recurrence of a bacterial infection or symptom associated therewith; (vii) reduce organ failure associated with a bacterial infection; (viii) reduce hospitalization of a subject having a bacterial infection; (ix) reduce hospitalization length of a subject having a bacterial infection; (x) increase the survival of a subject with a bacterial infection; (xi) eliminate a bacterial infection in a subject; (xii) inhibit or reduce a bacterial replication in a subject; and/or (xiii) enhance or improve the prophylactic or therapeutic effect(s) of another therapy.

“Subject” refers to an animal, in particular a mammal such as a primate (e.g. human).

“Essentially free,” as in “essentially free from” or “essentially free of,” means comprising less than a detectable level of a referenced material or comprising only unavoidable levels of a referenced material (trace amounts).

The word “substantially” does not exclude “completely” e.g. a composition which is “substantially free” from Y may be completely free from Y. “Substantially pure” refers to material which is at least 50% pure (i.e., free from contaminants), at least 90% pure, at least 95% pure, at least 98% pure, or at least 99% pure.

As is conventional, the designation “NH2” or “N-” refers to the N-terminus of an amino acid sequence and the designation “COOH” or “C-” refers to the C-terminus of an amino acid sequence.

“Internal”, “Interior” as used herein with respect to a protein, residue, or amino acid sequence means located between the N-terminus and the C-terminus.

“Fragment” is a nucleotide or polypeptide comprising “n” consecutive nucleic acids or amino acids, respectively, of the reference sequence and wherein “n” is any integer that is less than the total number of amino acids in the reference sequence. In certain embodiments, “n” is any integer between 1 and 100. In this way, a “fragment thereof” of a hypothetical 100 residue long reference sequence (SeqX) may consist of any 1 to 99 consecutive amino acids of SeqX. In certain embodiments, a fragment consists of 10, 20, 30, 40 or 50 contiguous amino acids of the full length sequence. Fragments may be readily obtained by removing “n” consecutive amino acids from either or both of the N-terminus and C-terminus of the full length reference polypeptide sequence. Fragments may be readily obtained by removing “n” consecutive nucleic acids fom either or both of the 3′ and 5′ ends of the nucleotide sequence that encodes the full length reference polypeptide sequence. An “immunogenic fragment” as used herein consists of “n” consecutive amino acids of an antigen sequence and is capable of eliciting an antibody or immune response in a mammal. Fragments of a polypeptide, for example, can be produced using techniques known in the art, e.g. recombinantly, by proteolytic digestion, or by chemical synthesis. Internal or terminal fragments of a polypeptide can be generated by removing one or more nucleic acids from the 3′ or 5′ end (for a terminal fragment) or by removing one or more nucleic acids from both 3′ and 5′ ends (for an internal fragment) of a nucleotide sequence that encodes the polypeptide's full length amino acid sequence.

“Operably linked” or “operatively linked” means linked so as to be “operational”, for example, the configuration of polynucleotide sequences for recombinant protein expression. In certain embodiments, “operably linked” refers to the art-recognized positioning of, e.g., nucleic acid components such that the intended function (e.g., expression) is achieved. A person with ordinary skill in the art will recognize that under certain circumstances (e.g., a cleavage site or purification tag), two or more components “operably linked” together are not necessarily adjacent to each other in the nucleic acid or amino acid sequence (contiguously linked). A coding sequence that is “operably linked” to a “control sequence” (e.g., a promoter, enhancer, or IRES) is ligated in such a way that expression of the coding sequence is under the influence or control of the control sequence. A person with ordinary skill in the art will recognize that a variety of configurations are functional and encompassed.

“Recombinant” means artificial or synthetic. In certain embodiments, “recombinant” indicates the referenced amino acid, polypeptide, conjugate, antibody, nucleic acid, polynucleotide, vector, cell, composition, or molecule was made by an artificial combination of two or more molecules (e.g., heterologous nucleic acid or amino acid sequences). Such artificial combination includes, without limitation, chemical synthesis and genetic engineering techniques. In certain embodiments, a “recombinant polypeptide” refers to a polypeptide that has been made using recombinant nucleic acids (nucleic acids introduced into a host cell). In certain embodiments, a recombinant nucleic acid is not heterologous (e.g., wherein the recombinant nucleic acid is a second copy of a nucleic acid innately present within a host cell). A “transgene” herein means a polynucleotide introduced into a cell, therefore a transgene is recombinant.

“Mutant” and “Modified” are given their well-understood and customary meanings and at least signify that the referenced molecule is altered (structure and/or function) as compared to control (e.g., wild type molecule or its naturally occurring counterpart) under comparable conditions or signify that the referenced numeric value is altered (increased or decreased) as compared to that of control under comparable conditions.

“Conservative” amino acid substitutions or mutations refer to the interchangeability of residues having similar side chains, and thus typically involves substitution of the amino acid in the polypeptide with amino acids within the same or similar defined class of amino acids. However, as used herein, in some embodiments, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or I can be conservatively mutated to either another aliphatic residue or to another non-polar residue. The table below shows exemplary conservative substitutions.

| Residue | Possible Conservative Mutations |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P | none |
| N, Q, S, T | Other polar |
| H, Y, W, F | Other aromatic (H, Y, W, F) |
| C | None |

“Isolated” or “purified” herein means a polypeptide, conjugate, antibody, polynucleotide, vector, cell, composition, or molecule in a form not found in nature. This includes, for example, a polypeptide, conjugate, antibody, polynucleotide, vector, cell, composition, or molecule having been separated from host cell or organism (including crude extracts) or otherwise removed from its natural environment. In certain embodiments, an isolated or purified protein is a protein essentially free from all other polypeptides with which the protein is innately associated (or innately in contact with). For example, “isolated PglL” or “purified PglL” includes the recombinant PglL protein essentially free from other periplasmic polypeptides that the PglL protein would otherwise be associated with (in contact with) inside the host cell. For example, an “isolated O-glycosylated modified carrier protein” or “purified O-glycosylated modified carrier protein” may have been separated from un-O-glycosylated modified carrier protein (e.g., following in vitro conjugation steps). In certain embodiments, “isolated” or “purified” also means a protein is not bound to an antibody or antibody fragment. In certain embodiments, an isolated or purified protein does not include a collection of the protein's components (sub-parts). For example, wherein the protein is a complex of protein components, an “isolated/purified complex” may not include a collection of the complex's components (unbound to each other) obtained after, for example, application of sodium dodecyl sulfate (SDS) or 2-Mercaptoethanol (both of which break down the bonds between protein components in a complex).

A “Pharamaceutical-grade” or “pharmaceutically acceptable” polypeptide, conjugate, antibody, polynucleotide, vector, cell, composition, or molecule is isolated, purified, or otherwise formulated to be essentially free from impurities (e.g., essentially free from components (e.g., naturally occurring components) which are unacceptably toxic to a subject to which the polypeptide, conjugate, antibody, polynucleotide, vector, cell, composition, or molecule may be administered). A pharmaceutical-grade polypeptide, conjugate, antibody, polynucleotide, vector, cell, composition, or molecule is not a crude polypeptide, conjugate, antibody, polynucleotide, vector, cell, composition, or molecule.

“Homologue(s)” as used herein means two or more molecules that, despite originating from a different genus or species of organism and/or having divergent structure, have essentially the same function. To denote similar functionality herein, “PglL” or “PilE” may be used to refer to oligosaccharyltransferases or pilin, respectively, even if alternate designations are used in the art (for example, “PaPglL” herein encompasses the oligosaccharyltransferase referred to as “PglO” from Neisseria gonorrhoeae and that is a known homologue of N. meningitidis PglL ([16], [17]; see also [14], [18], [19])).

“Endogenous” as used herein means the referenced two or more polypeptides, conjugates, antibodies, polynucleotides, vectors, cells, compositions, or molecules originate from the same species of organism, or, in the case of a synthetic or recombinant polypeptide for example, consists essentially of the structure and function as those that originate from the same species of organism. With respect to PglL, for example, “endogenous” refers to the relationship of the subject PglL to the subject pilin (or GlycoTag therefrom) and means that they both originate from the same species of organism, or consist essentially of the structure and function as those that originate from the same species of organism. As an example, a Neisseria meningitidis PglL is “endogenous” to N. meningitidis PilE (and in this way, a PglL may be said to be “endogenous to” the referenced pilin). As a further example, a Neisseria meningitidis PglL is “endogenous to” N. meningitidis cells (especially control or wild type N. meningitidis cells).

“Heterologous” as used herein means the referenced two or more things are not associated with each other in nature. In certain embodiments, a protein is “heterologous” to a cell if a comparable naturally occurring cell (e.g., wild type cell under comparable conditions) would not produce that protein. In certain embodiments, a periplasmic signal sequence is “heterologous” to a protein (or to the protein's amino acid sequence) because the comparable naturally occurring protein (e.g., wild type protein) would not be operatively linked to that signal sequence.

“Nucleic acid,” “nucleotide,” “polynucleotide” is used to refer to ribonucleic acid (RNA), deoxyribonucleic acid (DNA), a polyribonucleotide molecule, or a polydeoxyribonucleotide molecule whether or not modified, unmodified, or synthetic. Thus, polynucleotides as defined herein may include single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. Thus, DNAs or RNAs with backbones modified for stability or for other reasons are “polynucleotides” as that term is intended herein. DNAs or RNAs may be synthetic (including, without limitation, the nucleic acid subunits that together form the polynucleotide). Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides. Polynucleotides can be made by a variety of methods, including in vitro recombinant DNA-mediated techniques and by expression of DNAs in cells and organisms. Polynucleotides include genomic and plasmid nucleic acids. DNA includes, without limitation, genomic (nuclear) DNA having introns, e.g., as well as recombinant DNA such as cDNA (e.g., introns removed). RNA includes, without limitation, mRNA and tRNA. It is envisioned that codon optimization is utilized for any recombinant expression of a polynucleotide molecule of the present invention.

“Vector” refers to a vehicle by which nucleic acid molecules are contained and transferred from one environment to another or that facilitates the manipulation of a nucleic acid molecule. A vector may be, for example, a cloning vector, an expression vector, or a plasmid. Vectors include, for example, a BAC or a YAC vector. The term “expression vector” includes, without limitation, any vector, (e.g., a plasmid, cosmid or phage chromosome) containing a coding sequence suitable for expression by a cell (e.g., wherein the coding sequence is operatively linked to a transcriptional control element such as a promoter). A vector may comprise two or more nucleic acid molecules, in certain embodiments each of those two or more nucleic acid molecules comprises a nucleotide sequence that encodes a protein.

“Polypeptide” and “protein” are used interchangeably herein to refer to polymers of amino acids of any length. “Peptide” may be used to refer to a polymer of amino acids consisting of 1 to 50 amino acids. The polymer can be linear or branched, it can comprise modified amino acids, and it can be interrupted by non-amino acids. The terms also encompass an amino acid polymer that has been modified naturally or by intervention; for example, disulfide bond formation, glycosylation (except the O-glycosylation of modified carrier proteins), lipidation, acetylation, phosphorylation, amidation, derivatization by known protecting/blocking groups, proteolytic cleavage, modification by non-naturally occurring amino acids, or any other manipulation or modification, such as conjugation with a labeling component. Also included within the definition are, for example, polypeptides containing one or more analogs of an amino acid (including, for example, unnatural amino acids, etc.), as well as other modifications known in the art.

A “Glycan” is a large carbohydrate molecule containing smaller sugar molecules and in certain embodiments herein refers to the oligosaccharide chain of a “glycoprotein” (a protein comprising glycan(s) covalently attached to amino acid side chains). “O-glycan” or “O-linked-glycan” is used herein to reference a glycan that is covalently attached to a serine or threonine residue of another molecule (i.e., the glycan is engaged in o-linked glycosylation). Glycans may be immunogenic. [3].

“Reducing end” of an oligosaccharide or polysaccharide is the monosaccharide with a free anomeric carbon that is not involved in a glycosidic bond and is thus capable of converting to the open-chain form. The first sugar (“S-1”) herein is that comprising the reducing end and the second sugar (“S-2”) is that which is adjacent to S-1. The S-2 sugar may be attached to the S-1 sugar by, for example, an α-(1→3), β-(1→3), β-(1→4), or α-(1→6) linkage (see [3]).

“Antigen” or “immunogen” herein refer to a substance, typically a protein or glycan, which is capable of inducing an immune response in a subject. In certain embodiments, an antigen is a protein (e.g., a glycoprotein) that is “immunologically active,” meaning that once administered to a subject (either directly or by administering to the subject a nucleotide sequence or vector that encodes the protein) it is able to evoke an immune response of the humoral and/or cellular type directed against that protein. “O-antigens” consist of repeats of an oligosaccharide unit (O-unit), which generally has between two and six sugar residues. [20]. O-antigens are components of the outer-membrane of gram-negative bacteria. [20]. In certain embodiments, the glycan is an O-antigen.

“Adjuvants” are non-antigen substances that enhance the induction, magnitude, and/or longevity of an antigen's immunological effect.

“Conjugation” references the coupling of carrier protein to saccharide (e.g., by covalent bond).

“Conjugate” herein means two or more molecules (e.g., proteins) which are attached to each other. The two or molecules are optionally recombinant molecules and/or are heterologous to each other. In certain embodiments, the conjugate comprises two or more molecules, the first being a carrier protein, for example a modified carrier protein, and the remaining one or more molecules being glycans covalently attached to a serine or threonine residue of the carrier protein. In certain embodiments, a conjugate comprises a glycosylated carrier protein, such as an O-glycosylated carrier protein, including an O-glycosylated modified carrier protein. A conjugate may be the result of chemical conjugation or in vitro conjugation (bioconjugation).

“Antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, multispecific antibodies such as bispecific antibodies generated from at least two intact antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc. The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab′, F(ab′)2, and Fv fragments, linear antibodies, and single chain antibodies.

“Antibody response” means production of an anti-antigen antibody. “Inducing an antibody response” or “raising an antibody response” means stimulating in vivo the production of an anti-antigen antibody, e.g., an anti-O-antigen antibody or an anti-glycan-antibody.

“Identical” or percent “identity” as used in the context of two or more sequences is a reference to the number of nucleotides or amino acids which are the same over the entire length of the aligned sequence (for clarity, a conserved amino acid substitution in this context would not be “the same” but an analog of an amino acid, e.g., is “the same”). There are several known ways to calculate percent identity (see [21]). Unless stated otherwise, percentage identity “X” herein of a first amino acid sequence to a second sequence amino acid is calculated as (100×(Y/Z)), where “Y” is the number of “matches” (amino acid residues scored as identical matches in the alignment of the first and second sequences, as aligned by visual inspection or a particular sequence alignment program) and “Z” is the total number of aligned residues. Therefore, and unless stated otherwise, if the first amino acid sequence is shorter than the second amino acid sequence and percent identity is calculated over “the entire length of the sequence,” “Z” is equal to the length (in number of amino acids) of the first sequence.

The percent identity can be measured using sequence comparison software or algorithms or by visual inspection. Various algorithms and software are known in the art that can be used to obtain alignments of amino acid or nucleotide sequences. One such non-limiting example of a sequence alignment algorithm is the algorithm described in [22], as modified in [23], and incorporated into the NBLAST and XBLAST programs ([24]). In certain embodiments, Gapped BLAST can be used as described in [24]. BLAST-2, WU-BLAST-2 ([25], ALIGN, ALIGN-2 (Genentech, South San Francisco, Calif.) or Megalign (DNASTAR) are additional publicly available software programs that can be used to align sequences. In certain embodiments, the percent identity between two nucleotide sequences is determined using the GAP program in GCG software (e.g., using a NWSgapdna.CMP matrix and a gap weight of 40, 50, 60, 70, or 90 and a length weight of 1, 2, 3, 4, 5, or 6). In certain alternative embodiments, the GAP program in the GCG software package, which incorporates the algorithm of Needleman and Wunsch ([26]) can be used to determine the percent identity between two amino acid sequences (e.g., using either a Blossum 62 matrix or a PAM250 matrix, and a gap weight of 16, 14, 12, 10, 8, 6, or 4 and a length weight of 1, 2, 3, 4, 5). Alternatively, in certain embodiments, the percent identity between nucleotide or amino acid sequences is determined using the algorithm of Myers and Miller ([27]). For example, the percent identity can be determined using the ALIGN program (version 2.0) and using a PAM120 with residue table, a gap length penalty of 12 and a gap penalty of 4. Appropriate parameters for maximal alignment by particular alignment software can be determined by one skilled in the art. In certain embodiments, the default parameters of the alignment software are used.

As a non-limiting example, whether any particular polynucleotide or polypeptide has a certain percentage sequence identity (e.g., is at least 80% identical, at least 85% identical, at least 90% identical, and in some embodiments, at least 90%, 91%, 92%, 93%, 94%, 95%, 96%, 97%, 98%, or 99% identical) to a reference sequence can, be determined using known methods such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman (Advances in Applied Mathematics 2: 482 489 (1981)) to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical “over the full length of” the sequences being compared, such as the coding region of a nucleotide sequence for example.

“Numbered with respect to”, “as compared to”, “numbered according to” is used herein to reference a location in an amino acid sequence while not being limited to that referenced amino acid sequence. It would therefore be understood, for example, that residue “128 numbered with respect to SEQ ID NO: 140” may encompass 129 of SEQ ID NO: 145 as well as 128 of SEQ ID NO: 163 (demonstrated below).

SEQ_ID_NO_140
SAVTEYYLNHGEWPGNNTSAGVATS-SEIK------ 29
SEQ_ID_NO_145
SAVTGYYLNHGTWPKDNTSAGVASSPTDIK------ 30
SEQ_ID_NO_163
GAVTEYEADKGVFPTSNASAGVAAA-ADINGK---- 31

“Host cell” as used herein refers to a cell into which a molecule (usually a heterologous or non-native nucleic acid molecule) is, has been, or will be introduced. A host cell herein does not encompass a whole human organism (i.e., an “isolated host cell”).

PglL

Oligosaccharyltransferases (OSTs or OTases) are membrane-embedded enzymes that transfer oligosaccharides from a lipid carrier to a nascent protein (unlike glycosyltransferases in the cytoplasm, which assemble oligosaccharides by sequential action, OTases transfer glycan to protein en bloc [2]). O-linked glycosylation consists of the covalent attachment of a sugar molecule (a glycan) to a side-chain hydroxyl group of an amino acid residue (e.g. serine, or threonine) in the protein target (e.g., pilin). Pilin-glycosylation gene L (PglL) proteins from, for example Neisseria meningitidis, are OTases involved in O-linked glycosylation. In the periplasm of gram-negative bacteria, PglLs transfer the glycan from Und-PP-glycan to a pilin protein ([1]). Unlike PglB (N-glycosylation), PglL does not require a 2-acetamido group at position C-2 of the reducing end or a β1, 4 linkage between the first two sugars for activity and so is able to transfer virtually any glycan (Neisseria meningitidis PglL transfers, e.g., C. jejuni heptasaccharide, E. coli O7 antigen, E. coli K30 capsular structure, S. enterica O-antigen, and E. coli 016 peptidoglycan subunits to pilin in both E. coli and Salmonella cells) ([1], [3], [14], [16]). NmPglL and homologues thereof, such as PglL from Neisseria gonorrhoeae (called “PglO”, [6] and [19]) and PilO from Pseudomonas aeruginosa ([15]), are therefore substrate “promiscuous” (i.e., they have relaxed substrate specificity and so are able to transfer diverse oligo- and polysaccharides). [1] and [14] (per [3] and [16]). Neisseria meningitidis PglL (NmPglL) Homologues are described herein (see Examples) and known to the art: [17], [28], [18]).

“PglL OTase” herein encompasses Neisseria meningitidis PglL OTase as well as NmPglL OTase Homologues. Therefore, the term “PglL OTases” herein includes, for example, Neisseria meningitidis PglL (NmPglL) Oligosaccharyltransferase (OTase), Neisseria gonorrhoeae PglL (NgPglL) OTase, Neisseria lactamica 020-06 (NlPglL) OTase, Neisseria lactamica ATCC 23970 PglL (Nl_{ATCC23970PglL}) OTase, and Neisseria gonorrhoeae F62 PglL (Ng_{F62}PglL) OTase.

“PglL Glycan Substrate”, “PglL Substrate” as used herein is a reference to a glycan which is transferrable by a PglL Otase (i.e., a glycan that is a substrate of PglL). See [1], [14], [29], [3], [16]. In certain embodiments, the PglL Glycan Substrate is attached to a lipid-carrier (“lipid-carrier-linked PglL Glycan Substrate”). In certain embodiments, the lipid-carrier is undecaprenol-pyrophosphate (UndPP), dolichol-pyrophosphate, or a synthetic equivalent thereof. In certain embodiments, the lipid-carrier is UndPP. In certain embodiments, the glycan is a “UndPP-linked PglL Substrate”. It is envisioned that a lipid-carrier-linked glycan is membrane-bound within a gram-negative host cell. A lipid-carrier-linked PglL Glycan Substrate being membrane bound may be said to be located “at the periplasm.” In certain embodiments, a NmPglL Glycan Substrate, a NgPglL Glycan Substrate, a NlPglL Glycan Substrate, or a NsPglL Glycan Substrate is specified. In certain embodiments, the PglL Glycan Substrate comprises a glycan having a reducing end of Glucose, Galactose, Galactofuranose, Rhamnose, GlcNAc, GalNAc, FucNAc, DATDH, GATDH, HexNAc, deoxy HexNAc, diNAcBac, or Pse. In certain embodiments the glycan is immunogenic (e.g., an “immunogenic PglL Glycan Substrate”). In certain embodiments the glycan is an O-antigen (e.g., a “PglL O-antigen Substrate”). See [1], [14], [29], [16], [30], [15].

Recombinant expression of a Neisserial PglL within a heterologous host cell is described herein and is known by the art (see [10], [8], [9], [29] (e.g., Table 1), [11], [1], [14], [5], [3], [31]; all incorporated herein by reference in their entireties).

Carrier Proteins

“Carrier protein” as used herein means a protein suitable for use as a carrier protein in the production of bioconjugate vaccines (e.g., [32]). “Carrier protein” as used herein is distinct from a “lipid carrier” (or “lipid-linked-carrier”), the latter of which include, without limitation, undecaprenyl-pyrophosphate (UndPP).

A “modified carrier protein” as used herein means a carrier protein that is altered (in one or more way) as compared to wild type (i.e., a “modified carrier protein” excludes a wild type pilin protein). A modified carrier protein includes, without limitation, a carrier protein incorporating one or more GlycoTag, purification tag, deletion (e.g., of at least a part of the transmembrane domain), insertion, and/or mutation (e.g., AcrA mutation(s) ([33]). In certain embodiments, the modified carrier protein is altered as compared to a control carrier protein (e.g., wild type) such that the modified carrier protein may be an “acceptor” of the PglL Glycan Substrate (i.e., accept the PglL Glycan Substrate directly from PglL without pilin intermediate). In certain embodiments, one such modified carrier protein is altered by comprising one or more GlycoTags. In certain embodiments, one such modified carrier protein comprises one or more GlycoTags at its N-terminus, C-terminus, and/or interior residues. For clarity, “a modified carrier protein comprising a carrier protein having one or more GlycoTags at its N-terminus and/or C-terminus” means “a modified carrier protein comprising a carrier protein operably linked to one or more GlycoTags at its N-terminus and/or C-temrinus.”

In certain embodiments, the modified carrier protein is covalently coupled to a glycan, either directly (e.g., via an O-linked glycosidic bond) or indirectly (e.g., via a linker), wherein the coupling is at one or more of the GlycoTags. In further embodiments, the glycan is a PglL Glycan Substrate. In certain embodiments, the modified carrier protein is coupled to a Shigella glycan (e.g. a Shigella sonnei glycan (such as S. sonnei O-antigen), or e.g. a Shigella flexneri glycan (such as Shigella flexneri 2a CPS), or a Shigella dysenteriae glycan). In certain embodiments, the modified carrier protein is coupled to a Streptococcus glycan (e.g. Streptococcus pneumoniae (such as Streptococcus pneumoniae sp. 12F CPS, S. pneumoniae sp. 8 CPS, S. pneumoniae sp. 14 CPS, S. pneumoniae sp. 23A CPS, S. pneumoniae sp. 33F CPS, or S. pneumoniae sp. 22A CPS)).

“O-glycosylated modified carrier protein” means the modified carrier protein is glycosylated and, in particular, is engaged in O-linked glycosylation (e.g., a modified carrier protein that is O-linked to a PglL Glycan Substrate).

An O-glycosylated modified carrier protein may be directly or indirectly attached to two or more distinct immunogenic glycans and, in this way, useful for inducing an immune or antibody response to the two or more immunogenic glycans (i.e., multivalent).

Exemplary carrier proteins include, without limitation, detoxified Exotoxin A of P. aeruginosa (“EPA”; see, e.g., [4]), CRM197, maltose binding protein (MBP), Diphtheria toxoid (DT), Tetanus toxoid (TT), Tetanus Toxin C fragment (TTc), detoxified hemolysin A of S. aureus, clumping factor A, clumping factor B, E. coli FirmH, E. coli FirmHC, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin and detoxified variants thereof, C. jejuni Acriflavine resistance protein A (CjAcrA), E. coli Acriflavine resistance protein A (EcAcrA), Pseudomonas aeruginosa PcrV protein (PcrV), C. jejuni natural glycoproteins, S. pneumoniae NOX, S. pneumoniae PspA, S. pneumoniae PcpA, S. pneumoniae PhtD, S. pneumoniae PhtE, S. pneumoniae ply (e.g. detoxified ply), S. pneumoniae LytB, Haemophilus influenzae protein D (PD). [34], [35], [36]. In certain embodiments, the carrier protein is selected from the group consisting of CTB, TT, TTc, DT, CRM197, EPA, EcAcrA, CjAcrA, and PcrV. In certain embodiments, the carrier protein is selected from the group consisting of EPA, EcAcrA, CjAcrA, and PcrV. In certain embodiments, the carrier protein is EPA. In certain embodiments, the carrier protein is EcAcrA.

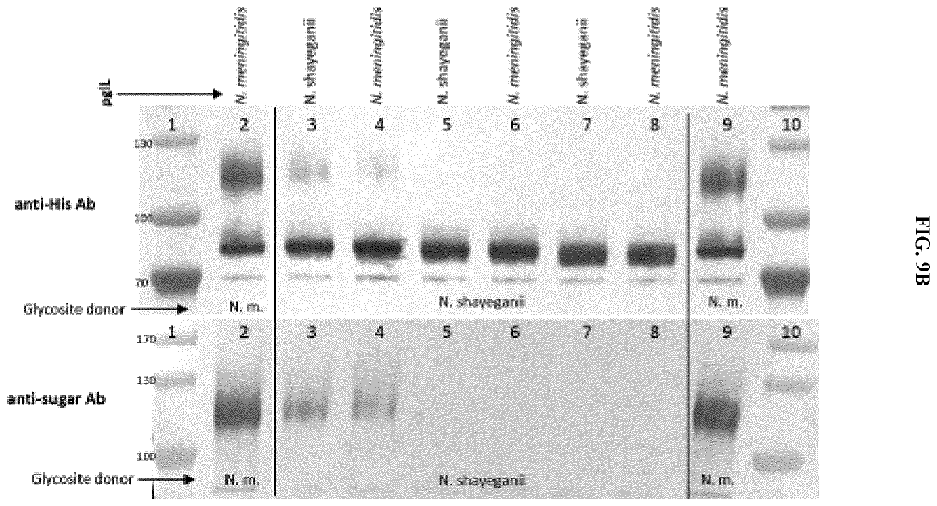

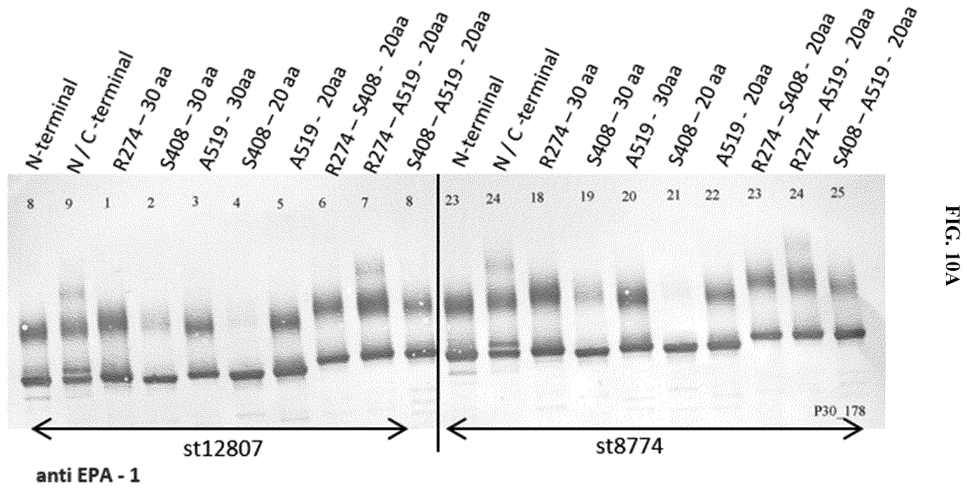

In certain embodiments, the carrier protein is protein D from Haemophilus influenzae (PD), for example, protein D sequence from FIG. 9 of [37] (FIG. 9a and 9b together, 364 amino acids). Inclusion of this protein in the immunogenic composition may provide a level of protection against Haemophilus influenzae related otitis media ([38]). The Protein D may be used as a full length protein or as a fragment (for example, Protein D may be as described in [39]). For example, a protein D sequence may comprise (or consist) of the protein D fragment as described in [37] lacking the 19 N-terminal amino acids from FIG. 9 of [37], optionally with the tripeptide MDP from NS1 fused to the N-terminal of said protein D fragment (348 amino acids). In one aspect, the protein D or fragment of protein D is unlipidated.

In an embodiment, the carrier protein is CRM197. CRM197 is a non-toxic form of the diphtheria toxin but is immunologically indistinguishable from the diphtheria toxin (DT). Genetically detoxified analogues of diphtheria toxin include CRM197 and other mutants described in U.S. Pat. Nos. 4,709,017, 5,843,711, 5,601,827, and 5,917,017. CRM197 is produced by C. diphtheriae infected by the nontoxigenic phase β197tox-created by nitrosoguanidine mutagenesis of the toxigenic carynephage b ([40]). The CRM197 protein has the same molecular weight as the diphtheria toxin but differs from it by a single base change in the structural gene. This leads to a glycine to glutamine change of amino acid at position 52 which makes fragment A unable to bind NAD and therefore non-toxic ([41], [42]).

In an embodiment, the carrier protein is Tetanus Toxoid (TT). Tetanus toxin is a single peptide of approximately 150 kDa, which consists of 1315 amino-acid residues. Tetanus-toxin may be cleaved by papain to yield two fragments; one of them, fragment C, is approximately 50 kDa. Fragment C of TT is described in [43].

In an embodiment, the carrier protein is dPly (detoxified pneumolysin). Pneumolysin (Ply) is a multifunctional toxin with a distinct cytolytic (hemolytic) and complement activation activities ([44]). The toxin is not secreted by pneumococci, but it is released upon lysis of pneumococci under the influence of autolysin. Its effects include e.g., the stimulation of the production of inflammatory cytokines by human monocytes, the inhibition of the beating of cilia on human respiratory epithelial, the decrease of bactericidal activity and migration of neutrophils, and in the lysis of red blood cells, which involves binding to cholesterol. Because it is a toxin, it needs to be detoxified (i.e., non-toxic to a human when provided at a dosage suitable for protection) before it can be administered in vivo. Expression and cloning of wild-type or native pneumolysin is known in the art. See, for example, [45], [46], and [47]. Detoxification of Ply can be conducted by chemical means, e.g., subject to formalin or glutaraldehyde treatment or a combination of both ([48], [49]). Such methods are known in the art for various toxins. Alternatively, Ply can be genetically detoxified (altered so that it is biologically inactive whilst still maintaining its immunogenic epitopes, e.g., [50], [51], and [52]. As used herein, it is understood that the term “Ply” encompasses mutated pneumolysin and detoxified pneumolysin (dPly) suitable for pharmaceutical use (i.e., non toxic).

Nucleic acids encoding the carrier protein can be introduced into a host cell for the production of a bioconjugate comprising a carrier protein. For use in in vivo bioconjugation within a gram-negative bacterium, carrier proteins are located within the periplasm. A carrier protein may be targeted to the periplasm by use of a periplasmic signal sequence. Periplasmic signal sequence structure and use (including their cleavage, codon optimization, and recombinant attachment to a heterologous protein) is known in the art. See, e.g., [53], [54], [5], and [34]. Codon optimization, generally, is also well known in the art and, unless stated otherwise (including Examples), it is envisioned that codon optimization is utilized for any recombinant expression of the present invention. See, e.g., [55], [56], [57], [58], [59] [60].

Signal sequences, including periplasmic signal sequences, are usually removed during translocation of the protein into, for example, the periplasm by signal peptidases (i.e., a mature protein is a protein from which at least the signal sequence has been removed). “Targeted to the periplasm” is used herein to acknowledge that signal sequences are usually removed. In this way, a protein which is “targeted to the periplasm” includes both the protein operably linked to the periplasmic signal sequence and the mature protein from which the periplasmic signal sequence has already been removed.

Periplasmic signal sequences are well known in the art. In certain embodiments, the periplasmic signal is that of Erwinia carotovorans pectatelyase B (pelB), E. coli outer membrane porin A (OmpA), E. coli disulfide oxidoreductase (DsbA), E. coli Tol-Pal cell envelop complex (TolB), E. coli maltose binding protein subunit (MalE), E. coli flagellin (Flgl), Heat-liable enterotoxin (LtIIb) (e.g., E. coli LtIlb), SipA (e.g., Streptococcus pyogenes SipA, Clostridium acidurici SipA, Bacillus amyloliquefaciens SipA), E. coli nickel-binding protein NikA (NikA), Bacillus sp. Endoxylanase (XynA), E. coli Heast Stable Enterotoxin II (STII), or E. coli alkaline phosphatase subunit (PhoA). [5]. In certain embodiments, the periplasmic signal sequence is PelB, OmpA, DsbA, TolB, or MalE. In certain embodiments, the periplasmic signal sequence is DsbA.

In certain embodiments, the carrier proteins comprise a “tag,” i.e., a sequence of amino acids that allows for the detection, isolation and/or identification of the carrier protein. For example, adding a tag to a carrier protein can be useful in the purification of that protein and, hence, the purification of a bioconjugate comprising the tagged carrier protein. Exemplary tags that can be used herein include, without limitation, histidine (HIS) tags (e.g., hexa histidine-tag, or 6×His-Tag), FLAG-TAG, and HA tags also strep tag, myc tag, or combinations thereof. In certain embodiments, the tags used herein are removable, e.g., removal by chemical agents or by enzymatic means, once they are no longer needed, such as after the protein has been purified.

A “purification tag” as used herein refers to a ligand that aids protein purification with, for example, size exclusion chromatography, ion exchange chromatography, and/or affinity chromatography. Purification tags and their use are well known to the art (see, e.g., [61], [62]) and may be, for example, poly-histidine (HIS), glutathione S-transferase (GST), c-Myc (Myc), hemagglutinin (HA), FLAG, or maltose binding protein (MBP). In certain embodiments, apurification tag is an epitope tag (which include, e.g., a histidine, FLAG, HA, Myc, V5, Green Fluorescent Protein (GFP), GSK, β-galactosidase (b-GAL), luciferase, Maltose Binding Protein (MBP), or Red Fluorescence Protein (RFP) tag). In certain embodiments, polypeptides are operably linked to one or more purification tags (including combinations of purification tags). A step of purifying, collecting, obtaining, or isolating a protein may therefore include size exclusion chromatography, ion exchange chromatography, or affinity chromatography. In certain embodiments, a step of purifying a modified carrier protein (or a conjugate comprising it), utilizes affinity chromatography and, for example, a σ28 affinity column or an affinity column comprising an antibody that binds the modified carrier protein or the conjugate comprising it (optionally by binding to the glycn). In a certain embodiment, a step of purifying a fusion protein comprising at least a modified carrier protein operably linked to a purification tag utilizes affinity chromatography and, for example, an affinity column that binds the purification tag.

GlycoTags

“GlycoTag” as used herein is a recombinant O-linked glycosylation site and consists of a fragment of a pilin amino acid sequence. In this way, the term “Glycotag” is used to refer to a recombinant amino acid sequence (i.e., separated from a wild type pilin) whereas “sequon” may be used to refer to that same sequence that is located within a wild type pilin (i.e., not separated from a wild type pilin).

The use of multiple GlycoTags within one carrier protein is envisioned (see Examples), optionally, multiple GlycoTags being adjacent to each other. Two or more GlycoTags may be separated by a “Amino Acid Linker” consisting of one or more amino acids, which can be, for example, one or more glycine ([63]), one or more serine, and/or combinations thereof (See [64]). An “amino acid linker” herein is a type of “linker”.

O-glycosylation efficiency of GlycoTags located at the N- or C-terminus of a carrier protein may be increased by flanking the GlycoTag (i.e., placing toward the N-terminus and/or toward the C-terminus of the GlycoTag) with one or more “Flanking Peptide” (a peptide comprising hydrophilic amino acids such as, for example, DPRNVGGDLD (residues 599-608 of SEQ ID NO: 1) or QPGKPPR (residues 628-634 of SEQ ID NO: 1)). [3]. Such Flanking Peptide may be adjacent to the GlycoTag (i.e., with no amino acids between the GlycoTag and the Flanking Peptide) or may have 1, 2, 3, 4, 5, 6, 7, 8, 9, or 10 amino acids between it and the GlycoTag. An insertion of two or more Flanking Peptides can be used. Flanking Peptides can be used to increase the O-glycosylation efficiency of shorter GlycoTags, such as those having the sequence SEQ ID NO: 142, 147, 151, or 164 (all 12 amino acids long).

Hydrophilic amino acids herein include arginine (R), lysine (K), aspartic acid (D), glutamic acid (E), glutamine (Q), asparagine (N), histidine (H), serine (S), threonine (T), tyrosine (Y), cysteine (C), and tryptophan (W).

Glycans

A glycan is any sugar that can be transferred (e.g, covalently attached) to a carrier protein. A glycan comprises monosaccharides, oligosaccharides and polysaccharides. An oligosaccharide is a glycan having 2 to 10 monosaccharides. A polysaccharide is a glycan having greater than 10 monosaccharides. Polysaccharides can be selected from the group consisting of O-antigens, capsules, and exopolysaccharides.

Glycans for use with the present invention are PglL Otase substrates. [1], [14], [29], [16], [30], and [15]. In certain embodiments, the glycan is operably linked to a lipid-carrier. In certain embodiments, the glycan can be, but is not limited to, hexoses, N-acetyl derivatives of hexoses, oligosaccharides, and polysaccharides. In certain embodiments, the monosaccharide at the reducing end of the glycan is a hexose or an N-acetyl derivative of a hexose. In a certain embodiment, the glycan comprises a hexose monosaccharide at its reducing end such as glucose, galactose, rhamnose, arabinotol, fucose or mannose. In certain embodiments, the hexose monosaccharide at the reducing end is glucose or galactose. In certain embodiments, the reducing end of the glycan is an N-acetyl derivative of hexose. In general, N-acetyl derivatives of hexose (or “hexose monosaccharide derivatives”) comprise an acetamido group at position 2. In certain embodiments, N-acetyl derivatives of hexose is selected from N-acetylglucosamine (GlcNAc), N-acetylhexosamine (HexNAc), deoxy HexNAc, and 2,4-diacetamido-2,4,6-trideoxyhexose (DATDH), N-acetylfucoseamine (FucNAc), and N-acetylquinovosamine (QuiNAc). In certain embodiments, the N-acetyl derivative of hexose is selected from N-acetylglucosamine (GlcNAc), N-acetylgalactosamine (GalNAc), N-acetylfucoseamine (FucNAc), 2,4-diacetarnido-2,4,6-trideoxyhexose (DATDH), glyceramido-acetamido trideoxyhexose (GATDH), and N-acetylhexosamine (HexNAc). In certain embodiments, the glycan has a reducing end of N,N-diacetylbacillosamine (diNAcBac) or Pseudaminic acid (Pse). In certain embodiments, the glycan is one that has a reducing end of Glucose, Galactose, arabinotol, fucose, mannose, Galactofuranose, Rhamnose, GlcNAc, GalNAc, FucNAc, DATDH, GATDH, HexNAc, deoxy HexNAc, QuiNAc, diNAcBac, or Pse. In certain embodiments, the glycan is one that has a reducing end of Glucose, Galactose, GlcNAc, GalNAc, FucNAc, DATDH, GATDH, HexNAc, deoxy HexNAc, or diNAcBac. In certain embodiments, the glycan is one that has a reducing end of Glucose, Galactose, Galactofuranose, Rhamnose, GlcNAc, GalNAc, FucNAc, DATDH, GATDH, or diNAcBac. In certain embodiments, the glycan is one that has a reducing end of Glucose, Galactose, GlcNAc, GalNAc, FucNAc, DATDH, GATDH, or diNAcBac. In certain embodiments, the glycan is one that has a reducing end selected from the group consisting of DATDH, GlcNAc, GalNAc, FucNAc, Galactose, and Glucose. In certain embodiments, the glycan is one that has a reducing end GlcNAc, GalNAc, FucNAc, or Glucose. In certain embodiments, the glycan is one that has a S-2 to S-1 reducing end of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine.

In certain embodiments, the glycan is endogenous to a Neisseria, Shigella, Salmonella, Streptococcus, Escherichia, Pseudomonas, Yersinia, Campylobacter, or Heliobacter cell. In certain embodiments, the glycan is endogenous to a Shigella, Salmonella, Escherichia, or Pseudomonas cell. In certain embodiments, the glycan is endogenous to a Shigella flexneri, Salmonella paratyphi, Salmonella enterica, or E. coli cell. In certain embodiments, the glycan is from C. jejuni, N. meningitidis, P. aeruginosa, S. enterica LT2, or E. coli. See [3], [29], [1], [14].

In certain embodiments, the glycan is an immunogenic glycan (an antigen). In certain embodiments, the glycan is an O-antigen. In certain embodiments, the glycan is an immunogenic O-antigen endogenous to a Neisseria, Shigella, Salmonella, Streptococcus, Escherichia, Pseudomonas, Yersinia, Campylobacter, or Heliobacter cell. In further embodiments, the PglL Glycan Substrate is a Shigella sonnei glycan antigen e.g. S. sonnei O-antigen, a Shigella flexneri glycan antigen e.g. Shigella flexneri 2a CPS, a Shigella dysenteriae glycan antigen, a Streptococcus pneumoniae glycan antigen e.g. Streptococcus pneumoniae sp. 12F CPS, S. pneumoniae sp. 8 CPS, S. pneumoniae sp. 14 CPS, S. pneumoniae sp. 23A CPS, S. pneumoniae sp. 33F CPS, or S. pneumoniae sp. 22A CPS. In certain embodiments, the glycan is a Streptococcus pneumoniae glycan having a reducing end of Glucose, Galactose, arabinotol, fucose, mannose, Galactofuranose, Rhamnose, GlcNAc, GalNAc, FucNAc, DATDH, GATDH, HexNAc, deoxy HexNAc, QuiNAc, diNAcBac, or Pse. In certain embodiments, the glycan is a Streptococcus pneumoniae glycan is one that has a S-2 to S-1 reducing end of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine. The CP gene clusters of all 90 S. pneumoniae serotypes have been sequenced by Sanger Institute (http://WorldWideWeb(www).sanger.ac.uk/Projects/S_pneumoniae/CPS/). Sequences are provided in NCBI as Genbank CR931632-CR931722. The capsular biosynthetic genes of S. pneumoniae are further described in Serotype 23A from Streptococcus pneumoniae strain 1196/45 (serotype 23a) as NCBI GenBank accession number: CR931683.1. Serotype 23B from Streptococcus pneumoniae strain 1039/41 as NCBI GenBank accession number: CR931684.1. Serotype 23F from Streptococcus pneumoniae strain Dr. Melchior as NCBI GenBank accession number: CR931685.1.

In certain embodiments, the glycan is an S. sonnei O-antigen. In certain embodiments, the S. sonnei O-antigen consists of a wbgT protein, a wbgU protein, a wzx protein, a wzy protein, a wbgV protein, a wbgW protein, a wbgX protein, a wbgY protein, and a wbgZ protein. In certain embodiments, the S. sonnei O-antigen consists of a wbgT protein having at least 90% identity to SEQ ID NO: 108, a wbgU protein having at least 90% identity to SEQ ID NO: 109, a wzx protein having at least 90% identity to SEQ ID NO: 110, a wzy protein having at least 90% identity to SEQ ID NO: 111, a wbgV protein having at least 90% identity to SEQ ID NO: 112, a wbgW protein having at least 90% identity to SEQ ID NO: 113, a wbgX protein having at least 90% identity to SEQ ID NO: 114, a wbgY protein having at least 90% identity to SEQ ID NO: 115, and a wbgZ protein having at least 90% identity to SEQ ID NO: 116).

Applications Thereof

Conjugation

As provided herein, the modified carrier proteins can be used for bioconjugation. In certain embodiments, the modified carrier proteins can be used for in vivo bioconjugation within a gram-negative bacterial host cell. In certain embodiments, the modified carrier proteins can be used for conjugate production by incubating the modified carrier protein with a Neisserial PglL and a PglL glycan substrate, optionally in a suitable buffer.

In Vivo Bioconjugation

In certain embodiments, O-glycosylated modified carrier proteins are produced using in vivo methods and systems. In certain embodiments, an O-glycosylated modified carrier protein (or bioconjugate) is made and then isolated from the periplasm of the host cell. In vivo conjugation (“bioconjugation”) of the present invention utilizes known methodologies for recombinant protein expression within a gram-negative bacterial cell and isolation therefrom, including sequence selection and optimization, vector design, cloning plasmids, culturing parameters, and periplasmic purification techniques. See, e.g., [65], [3], [5], [7], [8], [9], [10], [11], [1], [14], [4], [63], and [31]. Methods of producing bioconjugates using host cells are described in, for example, [66] and [67]. Bioconjugation offers advantages over in vitro chemical conjugation in that bioconjugation requires less chemicals for manufacture and is more consistent in terms of the final product generated.

Gram-negative bacterial cells for use with the present invention include, but are not limited to, a cell from the genera Neisseria, Shigella, Salmonella, Escherichia, Pseudomonas, Yersinia, Campylobacter, Vibrio, Klebsiella, or Helicobacter. In certain embodiments, the host cell is selected from the group consisting of Neisseria, Shigella, Salmonella, Escherichia, Pseudomonas, Yersinia, Campylobacter, and Helicobacter cells. In certain embodiments, the host cell is selected from the group consisting of Shigella, Salmonella, and Escherichia cells. In an embodiment, the gram-negative bacterial cell is classified as a Neisseria ssp., Shigella ssp., Salmonella ssp., Escherichia ssp., Pseudomonas ssp., Yersinia ssp., Campylobacter ssp., Vibrio ssp., Klebsiella ssp., or Helicobacter ssp. cell. The gram-negative bacterial host cell may be classified as a Neisserial ssp. cell other than Neisseria elongata. In a further embodiment, the gram-negative bacterial cell is a Shigella flexneri, Salmonella paratyphi, Salmonella enterica, E. coli, or Pseudomonas aeruginosa cell. In an embodiment, the host cell is selected from the group consisting of Shigella flexneri, Salmonella paratyphi, and Escherichia coli cells. In certain embodiments, the host cell is a Vibrio cholerae cell. In certain embodiments, the host cell is an Escherichia coli cell. In an embodiment, the gram-negative bacterial cell originated from E. coli strain K12, Top10, W3110, CLM24, BL21, SCM6 or SCM7. In certain embodiments, the host cell is a Shigella flexneri cell. In certain embodiments, the host cell is a Salmonella enterica cell. In an embodiment, the gram-negative bacterial cell originated from S. enterica strain SL3261, SL3749, SL326iδwaaL, or SL3749. In certain embodiments, the host cell is a Salmonella paratyphi cell. In certain embodiments, the host cell is a Pseudomonas aeruginosa cell. See [10], [8], [9], [29] at e.g. Table 1 and [11]; [3], [31], [5], [1], [14].

In certain embodiments, the gram-negative bacterial cell is modified such that the cell's endogenous (periplasmic) O-antigen ligase (or “endogenous PglL homologue”) is reduced (deficient or “knockdown”) or knocked-out (KO) in expression or function as compared to control (e.g., wild type). In certain embodiments, “reduction of endogenous PglL homologue” or “the endogenous PglL homologue is reduced” is used to mean a reduction (e.g., a knockdown), which encompasses a knock-out, of the expression or function of the endogenous PglL homologue. In that way, a gram-negative bacterial cell of the present invention may be deficient in its endogenous PglL homologue. For example, the WaaL gene of E. coli and that of Salmonella enterica are functional homologues of N. meningitidis PglL ([17], [28], and [68]). It is therefore envisioned that, for example, an Escherichia or Salmonella host cell for use with the present invention is modified such that the expression or function of WaaL is at least reduced as compared to a control (optionally wild type) Escherichia or Salmonella cell under essentially the same conditions. In certain embodiments, the host cell's endogenous PglL gene (e.g., the waaL gene) has been replaced by a heterologous nucleotide sequence encoding an oligosaccharyltransferase. Techniques for knocking down or knocking out an endogenous PglL homologue are known and include, for example, mutation or deletion of the gene encoding the endogenous PglL homologue. See the Examples and, e.g., [3]; see also [18].

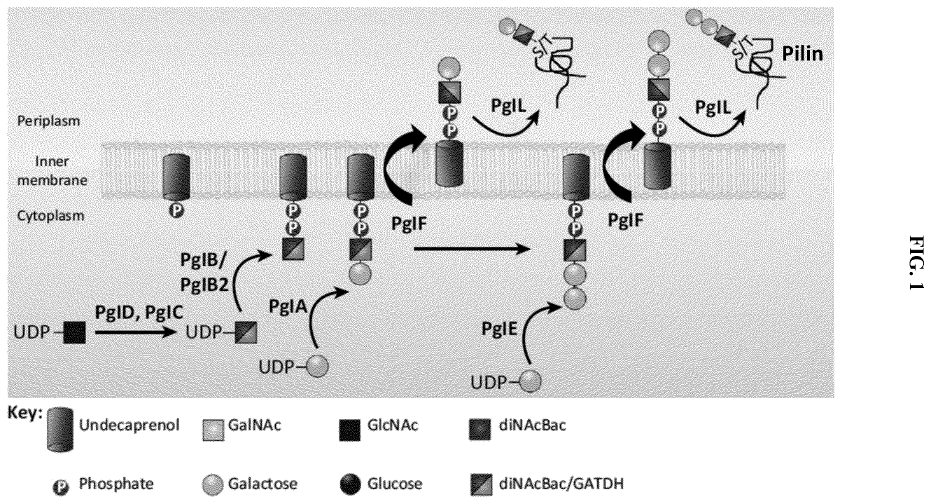

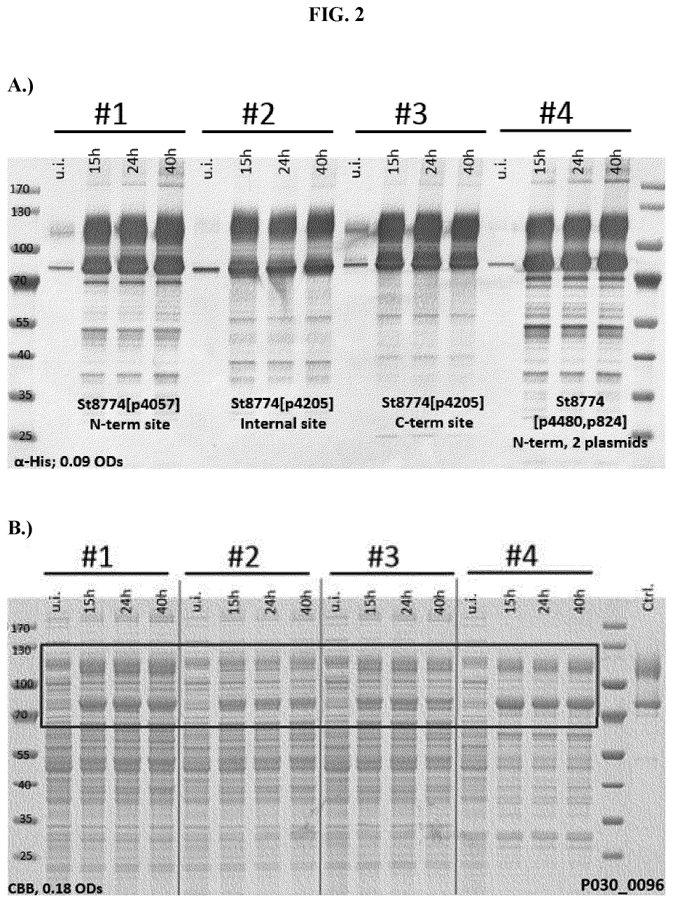

Host cells of the present invention may utilize endogenous or heterologous glycosyltransferases for sequential assembly of oligosaccharides in the cytosol (cytosolic glycosyltransferases). Such glycosyltransferases include, for example, Neisseria PglD, PglC, PglB/PglB2, and PglA shown at FIG. 1 and [2] (see also [103], particularly for Neisseria gonorrhoeae and see also [104], particularly for Neisseria elongata). The term “glycosyltransferases” is used herein as it is used by the art, to encompass what may be called “phospho-glycosyltransferases” (e.g., Neisserial PglB [103]). A gram-negative bacterial host cell may be modified to comprise a heterologous (e.g., bacterial or gram-negative bacterial) glycosyltransferase and optionally further modified to comprise reduced endogenous glycosyltransferase as compared to wild type (e.g. reduced expression of the corresponding endogenous glycosyltransferase). A host cell of the present invention may be chosen because its endogenous glycosyltransferases produce the target glycan or a host cell of the present invention may be engineered to express a heterologous glycosyltransferase(s) that assemble the target glycan (optionally further modified such that the host cell does not express the corresponding endogenous glycosyltransferase(s)). Such heterologous glycosyltransferases are not limited by origin so long as the glycosyltransferase assembles the target glycan structure. Activated sugar donors and their transporters or acceptors are also present. Glycosyltransferase selection and host cell engineering for target glycan assembly is common and well-known in the art ([105], [106]). In fact, “there is sufficient knowledge to predict the role of individual [glycosyltransferase] enzymes and assign them to specific pathways, allowing in silico prediction of the [glycosyltransferase]enzyme repertoire required to generate a particular glycan on a particular glycoconjugate” [106]. There are also tools publicly available by which a person with ordinary skill in the art may identify a glycosyltransferase which is capable of assembling the target glycan by searching for a glycosyltransferase having a particular function (i.e., searching the target glycan synthesis reaction) (e.g., Carbohydrate Active EnZYmes database WorldWideWeb.cazy.org/GlycosylTransferases) and/or by a structure-based search of the target glycan (e.g., the Bacterial Carbohydrate Structure DataBase (csdb.glycoscience.ru/bacterial) wherein glycosyltransferase information is provided, if previously published, for the glycan structure searched).

“O-glycosylation Machinery” is used to collectively reference the molecules (e.g. glycosyltransferases, flippases, polymerases, oligosaccharyltransferases including gene clusters and organelles) and processes for O-glycosylation which are well known to the art. See, e.g., [69], [3], [5], [31], [10], [8], [9], [29], [11]. In certain embodiments, a gram-negative bacterial host cell comprises O-glycosylation machinery that are endogenous, heterologous, or combinations thereof, to the host cell. In a fcurther embodiment, a gram-negative bacterial host cell comprises O-glycosylation machinery with the proviso that the cell's endogenous PglL or PglL homologue is reduced as compared to control. In a fcurther embodiment, a gram-negative bacterial host cell comprises endogenous O-glycosylation machinery with the proviso that the cell's endogenous PglL or PglL homologue is reduced as compared to control. In a certain embodiment the E. coli or S. enterica gram-negative host cell comprises endogenous O-glycosylation machinery with the proviso that the cell's PglL homologue WaaL is reduced as compared to control.

Again, codon optimization is well known in the art and, unless stated otherwise (including Examples), it is envisioned that codon optimization is utilized for any recombinant expression of the present invention.

The expression of the transgenes of the present invention can be under the control of a transcription control element (TCE) which includes, for example, a promoter. In certain embodiments, the transgene is under the control of a constitutive promoter or of an inducible promoter, which initiates transcription only when exposed to some particular external stimulus, such as, without limitation, antibiotics such as tetracycline, hormones such as ecdysone, or heavy metals. The promoter can also be specific to a particular cell-type, tissue or organ. Many suitable promoters and enhancers are known in the art, and any such suitable promoter or enhancer may be used for expression of the transgenes of the invention. Promoters for use with the present invention are known and include, without limitation, ParaBAD, arabinose, tac-promoter (Ptac), and constitutive promoters (including native constitutive promoters) ([4]; see also [10], [8], [9], [29], [11]). In certain embodiments, the promoter is a ParaBAD or arabinose promoter.

The incorporation of a nucleic acid molecule into a gram-negative bacterial cell can be performed using any number of techniques known in the art, including those for stable transfection or transformation of a nucleic acid molecule or vector into a host cell. See the references cited above and the techniques listed and described in [70]. Recombinant nucleic acids can be introduced into the host cells of the invention using methods such as electroporation, chemical transformation by heat shock, natural transformation, phage transduction, and conjugation. In certain embodiments, recombinant nucleic acids are introduced into a host cell using a plasmid (e.g. the recombinant nucleic acids are expressed in the host cell by a plasmid such as an expression vector). In another embodiment, recombinant nucleic acids are introduced into a host cell using the method of insertion described in [71].

Gram-negative bacterial cells incorporating the glycosyltransferases, modified carrier proteins, PglL Otases, or PglL Glycan Substrates of this invention can be grown using various methods known in the art, for example, grown in a broth culture. The modified carrier proteins or O-glycosylated modified carrier proteins produced by the cells can be isolated using various methods known in the art, for example, lectin affinity chromatography ([1]).

An O-glycosylated modified carrier protein may be purified (to remove host cell impurities and unglycosylated carrier protein) and optionally characterized by techniques known in the art (see, e.g., [4], [72]; see also [10], [8], [9], [29], and [11]). Purification of a bioconjugate may be by cell lysis (including, e.g., one or more centrifugation steps) followed by one or more isolation steps (including, e.g., one or more chromatography steps or a combination of fractionation, differential solubility, centrifugation, and/or chromatography steps). Said one or more chromatographic steps may comprise ion exchange, anionic exchange, affinity, and/or sizing column chromatography, such as Ni2+ affinity chromatography and/or size exclusion chromatography. In a certain embodiment, one or more chromatographic steps comprises ion exchange chromatography. Therefore, one or more of the purified polypeptides may be operably linked to a tag (a purification tag). For example, affinity column IMAC (Immobilized metal ion affinity chromatography) may be used to bind the poly-histidine tag operably linked to the carrier protein, followed by anion exchange chromatography and size exclusion chromatography (SEC). For example, purification of a bioconjugate may be by osmotic shock extraction followed by anionic and/or size exclusion chromatography ([7]); or by osmotic shock extraction followed by Ni-NTA affinity and fluoroapatite chromatography ([4]).

In Vitro Conjugation

To produce O-glycosylated modified carrier proteins in vitro, the PglL OTase can be incubated with the modified carrier protein and PglL glycan substrate in, for example, a buffer. In certain embodiments, the bugger has a pH of approximately 6 to approximately 8. In one aspect, the buffer may be phosphate buffer saline. In another aspect, the buffer may be Tris-HCl 50 mM, having a pH of 7.5.

In certain embodiments, chemical conjugation using known protocols is used (e.g., [73], [74], [75]). Thereby, a glycan may be covalently linked (either directly or through a linker) to an amino acid residue of a modified carrier protein. “Directly linked” herein means that the two entities are connected via a chemical bond, for example a covalent bond. “Indirectly linked” herein means that the two entities are connected via a linking moiety (“linker”) (as opposed to a direct covalent bond). In certain embodiments the linking moiety is adipic acid dihydrazide. In certain embodiments, the PglL glycan substrate is covalently linked to a modified carrier protein (directly or via a linker) through a chemical linkage obtainable using a chemical conjugation method selected from the group consisting of carbodiimide chemistry, reductive animation, cyanylation chemistry (for example CDAP chemistry), maleimide chemistry, hydrazide chemistry, ester chemistry, and N-hydroxysuccinimide chemistry. Conjugates can be prepared by direct reductive amination methods as described in, [76], [77]. Other methods are described in [78], [79], [80]. The conjugation method may alternatively rely on activation of the glycan with 1-cyano-4-dimethylamino pyridinium tetrafluoroborate (CDAP) to form a cyanate ester. Such conjugates are described in [81], [82], [83]. See also [84].

The glycosylated protein (i.e., conjugate) can then be purified, and optionally characterized, by techniques known in to art (see, e.g., [4], [72]; see also [8], [9], [10], [11]).

Conjugates

The O-glycosylated modified carrier proteins of the present invention can be used as therapeutic agents for the treatment of a number of diseases where an effective amount of the O-glycosylated modified carrier protein is administered to a subject in need of such treatment. The O-glycosylated modified carrier proteins of the present invention can also be used as a vaccine or in an immunogenic composition for the prevention of a disease when an effective amount of the O-glycosylated modifiec carriier protein is administered to a subject in need of such treatment. Thus, the methods described herein for producing of a number of different O-glycosylated modified crrier proteins will prove very useful in vaccinology.

“Homogeneity” means the variability of glycan length and possibly the number of glycosylation sites. Methods listed above can be used for this purpose. SE-HPLC allows the measurement of the hydrodynamic radius. Higher numbers of glycosylation sites in the carrier lead to higher variation in hydrodynamic radius compared to a carrier with less glycosylation sites. However, when single glycan chains are analyzed, they may be more homogenous due to the more controlled length. Glycan length is measured by hydrazinolysis, SDS PAGE, and CGE. In addition, homogeneity can also mean that certain glycosylation site usage patterns change to a broader/narrower range. These factors can be measured by Glycopeptide LC-MS/MS.

“Bioconjugate homogeneity” means the homogeneity of the attached sugar residues and can be assessed using methods that measure glycan length and hydrodynamic radius.

“Yield” is measured as carbohydrate amount derived from a liter of bacterial production culture grown in a bioreactor under controlled and optimized conditions. After purification of bioconjugate, the carbohydrate yields can be directly measured by either the anthrone assay or ELISA using carbohydrate specific antisera. Indirect measurements are possible by using the protein amount (measured by BCA, Lowry, or bardford assays) and the glycan length and structure to calculate a theoretical carbohydrate amount per gram of protein. In addition, yield can also be measured by drying the glycoprotein preparation from a volatile buffer and using a balance to measure the weight.

Analytical Methods

Various methods can be used to analyze the glycans and conjugates of the invention including, for example, SDS-PAGE or capillary gel electrophoresis. O-antigen polymer length is defined by the number of repeat units that are linearly assembled. This means that the typical ladder like pattern is a consequence of different repeat unit numbers that compose the glycan. Thus, two bands next to each other in SDS PAGE (or other techniques that separate by size) differ by only a single repeat unit. These discrete differences are exploited when analyzing glycoproteins for glycan size: the unglycosylated carrier protein and the bioconjugate with different polymer chain lengths separate according to their electrophoretic mobilities. The first detectable repeat unit number (n₁) and the average repeat unit number (n_{average}) present on a bioconjugate are measured. These parameters can be used to demonstrate batch to batch consistency or polysaccharide stability, for example.

In another embodiment, high mass MS and size exclusion HPLC could be applied to measure the size of the complete bioconjugates.

In another embodiment, an anthrone-sulfuric acid assay can be used to measure polysaccharide yields. See [85]. In another embodiment, a Methylpentose assay can be used to measure polysaccharide yields. See, e.g. [86].

Glycosylation Site Usage

Glycosylation site usage may be quantified by, for example, glycopeptide LC-MS/MS: conjugates are digested with protease(s), and the peptides are separated by a suitable chromatographic method (C18, Hydrophilic interaction HPLC HILIC, GlycoSepN columns, SE HPLC, AE HPLC), and the different peptides are identified using MS/MS. This method can be used with our without previous sugar chain shortening by chemical (smith degradation) or enzymatic methods. Quantification of glycopeptide peaks using UV detection at 215 to 280 nm allow relative determination of glycosylation site usage. In another embodiment, by size exclusion HPLC: Higher glycosylation site usage is reflected by a earlier elution time from a SE HPLC column.

Compositions

Compositions comprising a modified carrier protein are provided. In certain embodiments, the modified carrier protein is O-glycosylated. In certain embodiments, the glycan operably linked to the modified carrier protein is immunogenic and the composition is therefore an immunogenic composition.

An “immunogenic composition”, “vaccine composition,” or “pharmaceutical composition” is a preparation formulated to permit the biological activity of the active ingredient to be effective, and which contains no additional components which are unacceptably toxic to a subject to which the composition would be administered. Immunogenic, vaccine, or pharmaceutical compositions comprise pharmaceutical-grade active ingredients (e.g., pharmaceutical-grade antigen), therefore, the immunogenic, vaccine, or pharmaceutical compositions of the present invention are distinguished from any, e.g., naturally occurring composition. See [87]. In certain embodiments, the immunogenic, vaccine, or pharmaceutical composition is sterile. In certain embodiments, the composition is an immunogenic composition comprising an “immunogenic conjugate” (e.g., a modified carrier protein covalently linked to an immunogenic glycan). In certain embodiments, the immunogenic glycan is an O-antigen. Immunogenic compositions comprise an immunologically effective amount of the immunogenic glycan or immunogenic conjugate. An “immunologicaly effective amount” may be administered to an individual as a single dose or as part of a series. In certain embodiments, the immunogenic composition further comprises a pharmaceutically acceptable adjuvant, excipient, carrier, or diluent. Adjuvants, excipients, carriers, and diluents do not themselves induce an antibody or immune response, but rather they provide the technical effect of eliciting or enhancing an antibody or immune response to an antigen (e.g., an immunogenic glycan).

In an embodiment, the immunogenic compositions of the invention are monovalent formulations. In other embodiments, the immunogenic compositions of the invention are multivalent formulations, e.g. bivalent, trivalent, and tetravalent formulations. For example, a multivalent formulation comprises two or more immunogenic modified carrier proteins (e.g., a first immunogenic O-glycosylated modified carrier protein comprising a first immunogenic glycan and an at least second immunogenic O-glycosylated modified carrier protein comprising a second immunogenic glycan, optionally further comprising a third immunogenic O-glycosylated modified carrier protein comprising a third immunogenic glycan). In further embodiments, a multivalent immunogenic composition comprises an O-glycosylated modified carrier protein directly or indirectly attached to two or more distinct immunogenic glycans.

Also provided is a method of making an immunogenic composition comprising the step of mixing an immunogenic conjugate of the invention (e.g., an O-glycosylated modified carrier protein comprising an immunogenic glycan) with a pharmaceutically acceptable adjuvant, excipient, or diluent.

Provided are methods of inducing an antibody response in a mammal (e.g., a human mammal), comprising administering to the mammal an immunologically effective amount of an immunogenic composition of the present invention. Also provided are immunogenic compositions for use in inducing an antibody or immune response in a mammal. Provided are immunogenic compositions for the manufacture of a medicament for inducing an antibody or immune response in a mammal.

Streptococcus pneumoniae is a globally important encapsulated human pathogen. Streptococcus pneumoniae (S. pneumoniae, pneumococcus) is a Gram-positive bacterium responsible for considerable morbidity and mortality (particularly in infants and the elderly), causing invasive diseases such as bacteraemia and meningitis, pneumonia and other non-invasive diseases, such as acute otitis media. The major clinical syndromes caused by S. pneumoniae are widely recognized and discussed in standard medical textbooks. For instance, Invasive Pneumococcal Disease (IPD) is defined as any infection in which S. pneumoniae is isolated from the blood or another normally sterile site. Provided herein are immunogenic compositions for use in the treatment or prevention of a disease caused by Streptococcus pneumoniae infection, e.g. pneumonia, invasive pneumococcal disease (IPD), exacerbations of chronic obstructive pulmonary disease (eCOPD), otitis media, meningitis, bacteraemia, pneumonia and/or conjunctivitis. Provided are immunogenic compositions for use in inducing an immune response against a Streptococcus pneumoniae glycan in a mammal. Also provided are immunogenic compositions for inducing an antibody or immune response against a Streptococcus pneumoniae glycan in a mammal. Provided are immunogenic compositions for the manufacture of a medicament for inducing an antibody or immune response against a Streptococcus pneumoniae glycan in a mammal.

The disease caused by Streptococcus pneumoniae infection may be selected from pneumonia, invasive pneumococcal disease (IPD), exacerbations of chronic obstructive pulmonary disease (eCOPD), otitis media, meningitis, bacteraemia, pneumonia and/or conjunctivitis. Where the human mammal is an infant (defined as 0-2 years old in the context of the present invention), the disease may be selected from otitis media, meningitis, bacteraemia, pneumonia and/or conjunctivitis. In one aspect, where the human mammal is an infant (defined as 0-2 years old in the context of the present invention), the disease is selected from otitis media and/or pneumonia. Where the human mammal is elderly (i.e., 50 years or over in age, typically over 55 years and more generally over 60 years), the disease may be selected from pneumonia, invasive pneumococcal disease (IPD), and/or exacerbations of chronic obstructive pulmonary disease (eCOPD). In one aspect, where the human mammal is elderly, the disease is invasive pneumococcal disease (IPD). In another aspect, where the human mammal is elderly, the disease is exacerbations of chronic obstructive pulmonary disease (eCOPD).

Adjuvants

Adjuvants are non-antigen components used in immunogenic and vaccine compositions in order to enhance and modulate the immune or antibody response to the antigen. It is well recognized that an adjuvant enhances the induction, magnitude, and/or longevity of an antigen's immunological effect. An adjuvant is a compound that, when the compound is administered alone, does not generate an immune or antibody response to the antigen.

Immunogenic and vaccine compositions of the invention may comprise an adjuvant in addition to the antigen. In certain embodiments, the adjuvant is pharmaceutical-grade. An adjuvant may be administered before, concomitantly with, or after administration of an immunogenic or vaccine composition.

Specific examples of adjuvants include, but are not limited to, aluminum salts (alum) (such as aluminum hydroxide, aluminum phosphate, and aluminum sulfate), 3 De-O-acylated monophosphoryl lipid A (MPL), MF59, AS03, AS04, polysorbate 80 (TWEEN 80), imidazopyridine compounds (see [88]), imidazoquinoxaline compounds (see [89]), CpG ([90]) or unmethylated CpG containing oligonucleotides [91]), and saponins, such as QS21 (see [92]). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see [93]). In certain embodiments, the adjuvant is an oil-in-water emulsion (for example MF59, and AS03), liposomes (e.g., 3-o-desacyl-4′-Monophosphoryl Lipid A (MPL)) and/or saponins (e.g., QS21) (e.g., AS01), TLR2 agonist, TLR3 agonist, TLR4 agonist, TLR5 agonist, TLR6 agonist, TLR7 agonist, TLR8 agonist, TLR9 agonist, aluminium salt, nanoparticle, microparticle, ISCOMS, calcium fluoride, organic compound composite, or combinations thereof. See, e.g., [94], [95], and [96]). In a particular embodiment, the immunogenic or vaccine composition of the invention comprises an antigen and an adjuvant wherein the adjuvant is an oil-in-water emulsion (e.g., MF59, and AS03 and their respective subtypes including subtypes B and E), an aluminum salt (e.g., aluminum phosphate and aluminum hydroxide), a liposome, a saponin (e.g. QS21), an agonist of Toll-like receptors (TLRa) (e.g., TLR4a and TLR7a), or a combination thereof (e.g., Alum-TLR7a ([97]). By “TLR agonist” it is meant a component which is capable of causing a signaling response through a TLR signaling pathway, either as a direct ligand or indirectly through generation of endogenous or exogenous ligand ([98]). A TLR4 agonist, for example, is capable of causing a signalling response through a TLR-4 signalling pathway. A suitable example of a TLR-4 agonist is a lipopolysaccharide, suitably a non-toxic derivative of lipid A, particularly monophosphoryl lipid A or more particularly 3-Deacylated monophoshoryl lipid A (3D-MPL). In certain embodiments, the immunogenic or vaccine composition comprises one or more adjuvants.

In certain embodiments, the adjuvant is Monophosphoryl lipid A (such as 3-de-O-acylated monophosphoryl lipid A (3D-MPL)) or a derivative thereof, or a combination of monophosphoryl lipid A together with either an aluminium salt (e.g., aluminium phosphate or aluminium hydroxide) or an oil-in-water emulsion. In certain embodiments, the adjuvant comprises a formulation of QS21, 3D-MPL and tocopherol in an oil in water emulsion ([99]).

Excipients

Pharmaceutically acceptable excipients can be selected by those of skill in the art. For example, a pharmaceutically acceptable excipient may be a buffer, such as Tris (trimethamine), phosphate (e.g. sodium phosphate, sucrose phosphate glutamate), acetate, borate (e.g. sodium borate), citrate, glycine, histidine and succinate (e.g. sodium succinate), suitably sodium chloride, histidine, sodium phosphate or sodium succinate. A pharmaceutically acceptable excipient may include a salt, for example sodium chloride, potassium chloride or magnesium chloride. Optionally, a pharmaceutically acceptable excipient contains at least one component that stabilizes solubility and/or stability. Examples of solubilizing/stabilizing agents include detergents, for example, laurel sarcosine and/or polysorbate (e.g. TWEEN 80 (Polysorbate-80)). Examples of stabilizing agents also include poloxamer (e.g. poloxamer 124, poloxamer 188, poloxamer 237, poloxamer 338 and poloxamer 407). A phamaceutically acceptable excipient may include a non-ionic surfactant, for example polyoxyethylene sorbitan fatty acid esters, TWEEN 80 (Polysorbate-80), TWEEN 60 (Polysorbate-60), TWEEN 40 (Polysorbate-40) and TWEEN 20 (Polysorbate-20), or polyoxyethylene alkyl ethers (suitably polysorbate-80). Alternative solubilizing/stabilizing agents include arginine, and glass forming polyols (such as sucrose, trehalose and the like). A pharmaceutically excipient may be a preservative, for example phenol, 2-phenoxyethanol, or thiomersal. Other pharmaceutically acceptable excipients include sugars (e.g. lactose, sucrose), and proteins (e.g. gelatine and albumin). Pharmaceutically acceptable excipients for use with the present invention include saline solutions, aqueous dextrose and glycerol solutions (also referred to as “carriers” or “fillers” in the art). Numerous pharmaceutically acceptable excipients are described, for example, in [100].

Immunogenic compositions if the invention may also comprise diluents such as saline, and glycerol. Additionally, immunogenic compositions may comprise auxiliary substances such as wetting agents, emulsifying agents, pH buffering substances, and/or polyols.

Immunogenic compositions if the invention may also comprise one or more salts, e.g. sodium chloride, calcium chloride, sodium phosphate, monosodium glutamate, and aluminum salts (e.g. aluminum hydroxide, aluminum phosphate, alum (potassium aluminum sulfate), or a mixture of such aluminum salts).

Immunogenic compositions if the invention may also comprise a preservative, e.g. a mercury derivative thimerosal or 2-phenoxyethanol. In an embodiment, the immunogenic composition of the invention comprises 0.001% to 0.01% thimerosal. In an embodiment, the immunogenic composition of the invention comprises 0.001% to 0.01% 2-phenoxyethanol.

Immunogenic compositions if the invention may also comprise a detergent e.g. polysorbate, such as TWEEN 80 (Polysorbate 80). Detergents may be present at low levels e.g. <0.01%, but higher levels have been suggested for stabilising antigen formulations e.g. up to 10%.

Administration

Immunogenic compositions or vaccines of the invention may be used to induce an immune or antibody response and/or protect or treat a mammal susceptible to infection, by administering said immunogenic composition or vaccine composition to said mammal via systemic or mucosal route. These administrations may include injection via the intramuscular (IM), intraperitoneal, intradermal (ID) or subcutaneous routes; or via mucosal administration to the oral/alimentary, respiratory, genitourinary tracts. For example, intranasal (IN) administration may be used. Although the immunogenic composition or vaccine of the invention may be administered as a single dose, components thereof may also be co-administered together at the same time or at different times. For co-administration, the optional adjuvant, for example, may be present in any or all of the different administrations, however in one particular aspect of the invention it is present in combination with the immunogenic O-glycosylated modified carrier protein. In addition to a single route of administration, two different routes of administration may be used. Following an initial vaccination, subjects may receive one or several booster immunizations adequately spaced.

### EXAMPLES

It is understood that the examples and embodiments described herein are for illustrative purposes only and that various modifications or changes in light thereof will be suggested to persons skilled in the art and are to be included within the spirit and purview of this application.

### Example 1

### Materials and Methods

Escherichia coli deficient in O-antigen lipopolysaccharide ligase gene waaL (E. coli W3110 ΔwaaL, ΔwecA-wzzE, ΔO16::wbgT-wbgZ cluster of P. shigelloides O17 (S. sonnei) (“E. coli W3110ΔwaaL” hereafter)) containing a chromosomal copy of a polysaccharide biosynthesis cluster (0-antigen or capsular polysaccharide) as well as two plasmids expressing PglL and a modified carrier protein was used. A single colony was inoculated in 50 ml TBdev medium [yeast extract 24 g/L, soy peptone 12 g/L, glycerol 100% 4.6 mL/L, K₂HPO₄ 12.5 g/L, KH₂PO₄ 2.3 g/L, MgCl₂x6H₂O 2.03 g/L) and grown at 30° C. to an OD of 0.8. At this point, 0.1 mM IPTG and 0.1% arabinose were added as inducers. The culture was further incubated o/n and harvested for further analysis (see [00119]). In case of bioreactor evaluation, a 50 mL (uninduced) o/n culture was used to inoculate a 11 culture in a 21 bioreactor. The bioreactor was stirred with 500-1000 rpm, pH was kept at 7.2 by auto-controlled addition of either 2 M KOH or 20% H₃PO₄ and the cultivation temperature was set at 30° C. The level of dissolved oxygen (pO2) was kept at 10% oxygen. In batch phase cells were grown in a TBdev medium as described above but containing glycerol at 50 g/L. As feed medium TBdev supplemented with 250 g/L glycerol and 0.1% IPTG (one-plasmid system) or 0.1% IPTG and 2.5% arabinose (two-plasmid system) was used. Induction with 0.1 mM IPTG (one-plasmid system) and 0.1 mM IPTG and 0.1% arabinose (2-plasmid system) was done at OD=35, prior to starting the fed-batch phase of growth. A linear feed rate was sustained for 24 h, followed by a 16 h starvation period. The bioreactor culture was harvested after a total of ≈40 h cultivation, when it should have reached an OD600 of 80.

The production process was analyzed by Coomassie brilliant blue staining or Western blot as described previously ([101]). After being blotted on nitrocellulose membrane, the sample was immunostained with the either anti-His, anti-glycan or anti-carrier-protein. Anti-rabbit IgG-HRP (Biorad) was used as secondary antibody. Detection was carried out with ECL™ Western Blotting Detection Reagents (Amersham Biosciences, Little Chalfont Buchinghamshire).

For periplasmic protein extraction, the cells were harvested by centrifugation for 20 min at 10,000 g and resuspended in 1 volume 0.9% NaCl. The cells were pelleted by centrifugation during 25-30 min at 7,000 g. The cells were resuspended in Suspension Buffer (25% Sucrose, 100 mM EDTA 200 mM Tris HCl pH 8.5, 250 OD/ml) and the suspension was incubated under stirring at 4-8° C. during 30 min. The suspension was centrifuged at 4-8° C. during 30 min at 7,000-10,000 g. The supernatant was discarded, the cells were resuspended in the same volume ice cold 20 mM Tris HCl pH 8.5 and incubated under stirring at 4-8° C. during 30 min. The spheroblasts were centrifuged at 4-8° C. during 25-30 min at 10,000 g, the supernatant was collected and passed through a 0.2 g membrane. Periplasmic extract was loaded on a 7.5% SDS-PAGE, and stained with Coomasie for identification.

For bioconjugate purification, the supernatant containing periplasmic proteins obtained from 100,000 OD of cells was loaded on a Source Q anionic exchange column (XK 26/40≈180 ml bed material) equilibrated with buffer A (20 mM Tris HCl pH 8.0). After washing with 5 column volumes (CV) buffer A, the proteins were eluted with a linear gradient of 15 CV to 50% buffer B (20 mM Tris HCl+1M NaCl pH 8.0) and then 2 CV to 100% buffer B. Protein were analyzed by SDS-PAGE and stained by Coomassie. Bioconjugate may elute at conductivity between 6-17 mS. The sample was concentrated 10 times and the buffer was exchanged to 20 mM Tris HCl pH 8.0.

Bioconjugate was loaded on a Source Q column (XK 16/20˜28 ml bed material) equilibrated with buffer A: 20 mM Tris HCl pH 8.0. The identical gradient that was used above was used to elute the bioconjugate. Protein were analyzed by SDS-PAGE and stained by Coomassie. Normally the bioconjugate elutes at conductivity between 6-17 mS. The sample was concentrated 10 times and the buffer was exchanged to 20 mM Tris HCl pH 8.0.

Bioconjugate was loaded on Superdex 200 (Hi Load 26/60, prep grade) that was equilibrated with 20 mM Tris HCl pH 8.0. Protein fractions from Superdex 200 column were analyzed by SDS-PAGE and stained by Coomassie stained.

Bioconjugates from different purification steps were analyzed by SDS-PAGE and stained by Coomassie. Bioconjugate is purified to more than 98% purity using the process. Bioconjugate can be successfully produced using this technology.

### Carrier Protein Optimization

Pseudomonas exotoxin A (EPA) carrier protein (SEQ ID NO: 1) was modified to incorporate one or more GlycoTags from Neisseria meningitidis pilin PilE (wild type sequence provided as SEQ ID NO: 137) (for methods see [29]; [6]; [4]; and [31], all incorporated herein by reference in their entireties). Recombinant EPA (rEPA, SEQ ID NO: 1) was modified to make three other recombinant EPA proteins:
the first having been modified to incorporate, at its N-terminus, the NmPilE GlycoTag SEQ ID NO: 140 (corresponding to residues 45-73 of SEQ ID NO: 137); twenty-nine (29) amino acid long) (rEPA1, SEQ ID NO: 51).
The second having been modified to incorporate, at internal residue A375 with respect to SEQ ID NO: 1 ( ), the NmPilE GlycoTag SEQ ID NO: 140 (rEPA2, SEQ ID NO: 53).
The third having been modified to incorporate, at its C-terminus, the NmPilE GlycoTag SEQ ID NO: 140 (rEPA3, SEQ ID NO: 55).

### Technical Feasibility

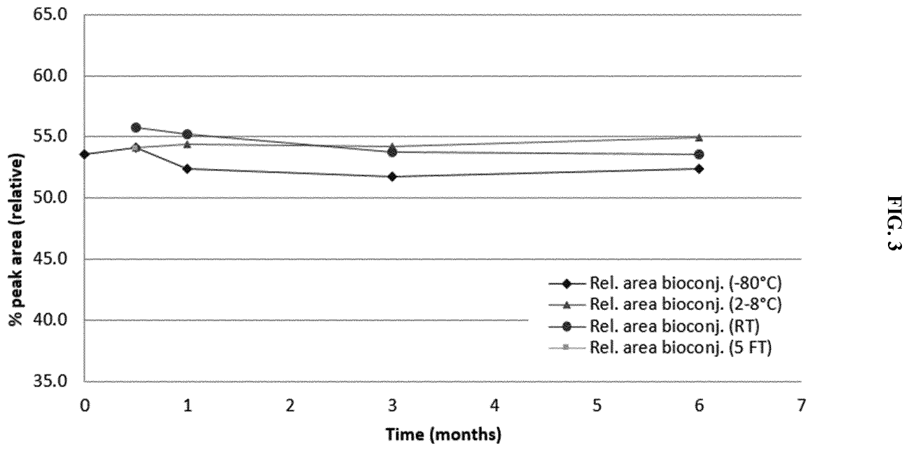

Neisseria meningitidis PglL (NmPglL) (polynucleotide sequence SEQ ID NO 8, encoding amino acid sequence SEQ ID NO: 9), Shigella sonnei O-antigen gene cluster (polynucleotide sequence SEQ ID NO: 6, encoding amino acid sequences SEQ ID NO: 208-216), and one of carrier proteins rEPA1, rEPA2, and rEPA3 (operatively linked to a DsbA periplasmic signal sequence (SEQ ID NO: 5, encoding SEQ ID NO: 4)) were introduced into Escherichia coli W3110 deficient in O-antigen lipopolysaccharide ligase gene waaL (E. coli W3110ΔwaaL). Three cell lots were made, one for each of rEPA1-rEPA3. Coomassie blue staining and Western blot assays confirmed that NmPglL efficiently transferred lipid-carrier-linked S. sonnei O-antigen to each of rEPA1, rEPA2, and rEPA3 (corresponding to #1-#4, respectively, in FIG. 2). Transfer was observed in either one plasmid systems (i.e. PglL and rEPA carrier combined in one plasmid (#1-#3 FIG. 2A and FIG. 2B) or in a two plasmid system (i.e. PglL and EPA encoded on two separate plasmids, IPTG and arabinose inducible (#4 FIG. 2A and FIG. 2B). Mass spectrometry confirmed that, when bound to rEPA1, the S. sonnei O-antigen is intact and its structure maintained. Twenty-one (21) to twenty-five (25) repeat units (below) have been determined to be attached to the rEPA1.

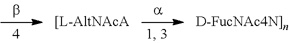

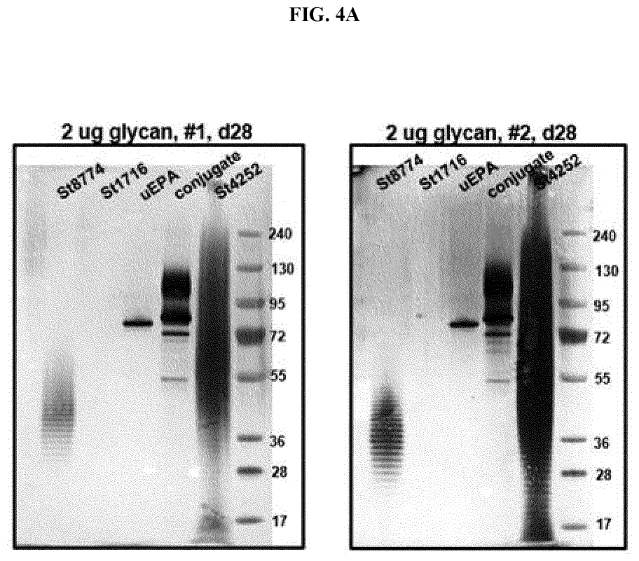

The stability of the rEPA1-S. sonnei O-antigen bioconjugate was studied at three different temperatures (−80° C., 2-8° C., and room temperature (RT) 20-25° C.) for a time of six months. Additionally, five freeze/thaw cycles (5 FT) on purified rEPA1-S. sonnei O-antigen were performed. SEC-HPLC readouts of samples taken at zero months, two weeks, one month, three months, and six months revealed that the rEPA1-S. sonnei O-antigen bioconjugate peak area was constant over time FIG. 3. The same was observed for the sample subjected to five freeze/thaw cycles. No degradation products were observed and only minor aggregation was observed. The bioconjugate had good stability overtime. FIG. 3.

### Immunogenicity

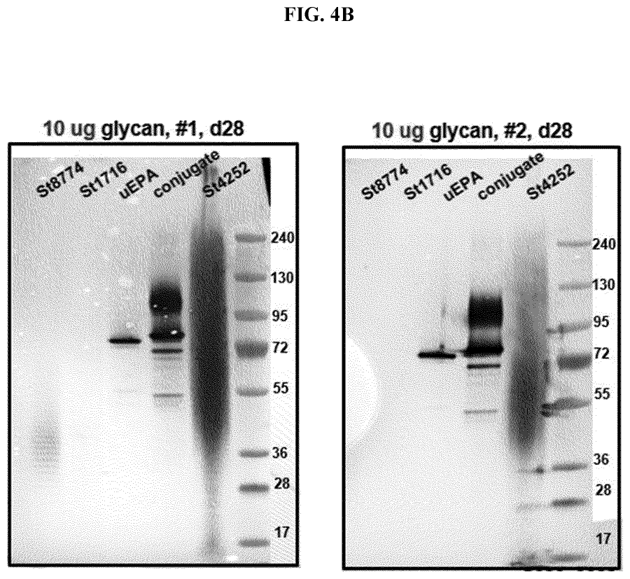

To evaluate the immunogenicity of the rEPA1-S. sonnei O-antigen bioconjugate, four female New Zealand White Rabbits (age 3-4 months) were divided into two groups (two rabbits per group) and subcutaneously injected at zero, seven, ten, and eighteen days with a bioconjugate composition comprising 2 μg of sugar, 40 μg of protein, and non-Freund's adjuvant (Group 1) or 10 μg of sugar, 200 μg of protein, and non-Freund's adjuvant (Group 2). Bleeds occurred at zero, twenty-one, and twenty-eight days. Western blot of the blood samples taken at twenty-eight days revealed that antibodies against S. sonnei O-antigen and EPA were generated in all subjects (FIG. 4), with the 2 μg dose (FIG. 4A) inducing a better antibody response than the 10 μg dose (FIG. 4B). These results show that the NmPglL mediated rEPA1-S. sonnei O-antigen bioconjugate is immunogenic in rabbits.

### Example 2

### Carrier Protein Versatility

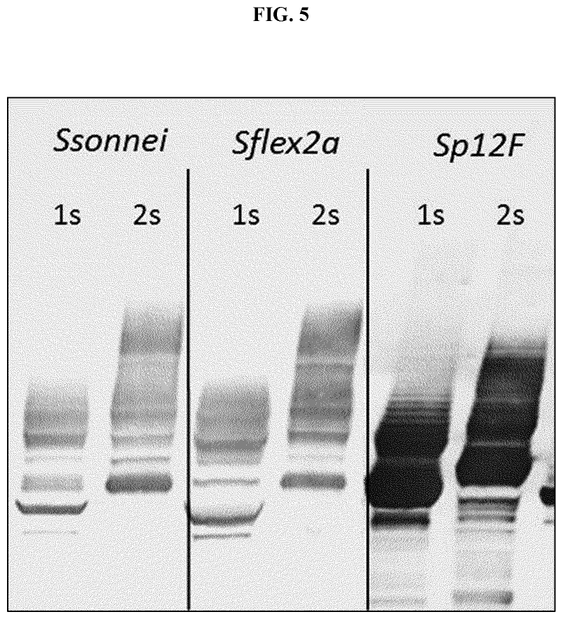

To evaluate the technical feasibility of using multiple GlycoTags on the carrier protein, EPA was modified to incorporate either one or two copies of the NmPilE GlycoTag having the sequence SEQ ID NO: 9. For EPA incorporating only one copy of the GlycoTag, it was located at the N-terminus (rEPA1). For EPA incorporating two copies of the GlycoTag SEQ ID NO: 140, the first GlycoTag was located at the N-terminus and the second was located at the C-terminus (rEPA43, SEQ ID NO: 135). Neisseria meningitidis PglL (NmPglL) was applied to rEPA1 or rEPA43 in the presence of one of three distinct lipid-carrier-linked polysaccharides: S. sonnei O-antigen, S. flexneri 2a CPS, or Streptococcus pneumoniae 12F CPS. NmPglL transferred each of S. sonnei O-antigen, S. flexneri 2a CPS, and Streptococcus pneumoniae 12F CPS onto rEPA1 and rEPA43 (FIG. 5).

These results show that a carrier protein modified to incorporate more than one GlycoTag may be used for in vivo bioconjugation.

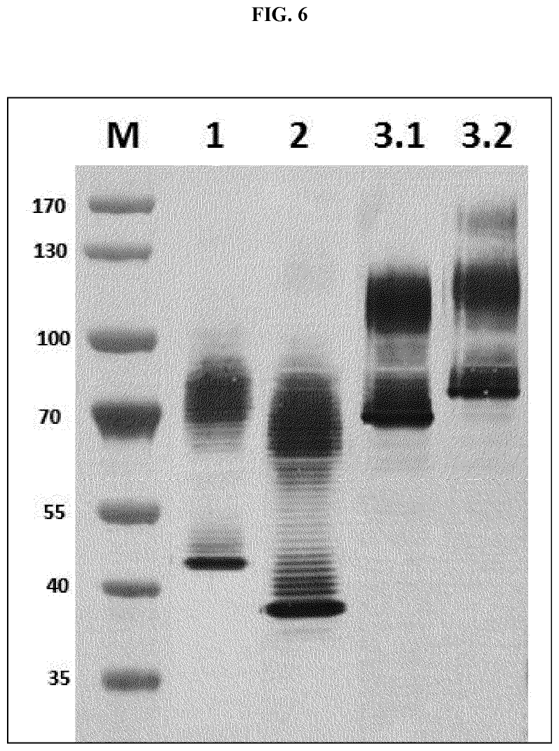

To evaluate the versatility of NmPglL toward carrier protein, known carrier proteins AcrA, PcrV, and Crm197 were also modified as above to incorporate one copy of the NmPilE GlycoTag having the sequence SEQ ID NO: 140. For modified AcrA (mAcrA), a pelB signal sequence (residues 1-22 of SEQ ID NO: 198) was operably linked to the N-terminus of the AcrA sequence, the GlycoTag SEQ ID NO: 140 was operably linked to the C-terminus of AcrA, and a 6×His-tag was operably linked to the C-terminus of the GlycoTag (SEQ ID NO: 199 for mAcrA). For modified PcrV (mPcrV), a LtIIb signal sequence (residues 1-23 of SEQ ID NIO: 202) was operably linked to the N-terminus of the PcrV sequence, the GlycoTag SEQ ID NO: 140 was operably linked to the C-terminus of PcrV, and a 6×His-tag was operably linked to the C-terminus of the GlycoTag (SEQ ID NO: 202 for mPcrV). For a first modified Crm197 (mCrm197), a DsbA signal sequence (SEQ ID NO: 4) was operably linked to the N-terminus of the Crm197 sequence, the GlycoTag SEQ ID NO: 140 was operably linked to the C-terminus of Crm197, and a 6×His-tag was operably linked to the C-terminus of the GlycoTag (SEQ ID NO: 204 for mCrm197). For a second modified Crm197 (m2Crm197, SEQ ID NO: 207), a DsbA signal sequence (SEQ ID NO: 4) was operably linked to the N-terminus of the GlycoTag sequence SEQ ID NO: 140, which were together operably linked to the N-terminus of the Crm197 sequence; the GlycoTag SEQ ID NO: 140 was also operably linked to the C-terminus of Crm197, and a 6×His-tag was operably linked to the C-terminus of the GlycoTag (see m2Crm197 sequence SEQ ID NO: 207). NmPglL, S. sonnei O-antigen, and one of mAcrA, mPcrV, mCrm197, and m2Crm197 were operatively introduced into E. coli W3110ΔwaaL. In this way, NmPglL contacted lipid-carrier-linked S. sonnei O-antigen in the presence of mAcrA, mPcrV, mCrm197, or m2Crm197. NmPglL transferred S. sonnei O-antigen onto mAcrA, mPcrV, mCrm197, and m2Crm197 (FIG. 6).

### PglL Substrate Versatility

To evaluate the substrate versatility of NmPglL, a polysaccharide gene cluster (i.e., nucleotide sequence) encoding a Pneumococcal capsular polysaccharide (CPSs) from one of each of serotypes Sp8, Sp12F, Sp14, Sp22A, Sp23A, and Sp33F was chromosomally introduced (Table 1) into E. coli W3110ΔwaaL. NmPglL, and rEPA1 or rEPA43 nucleotide sequences (Example 1 above) were also operatively introduced into each of the E. coli W3110ΔwaaL cells. Twelve recombinant host cells were made, six incorporating one of each of the six different Pneumococcal CPSes and rEPA1, and another six incorporating one of each of the six different Pneumococcal CPSes and rEPA43. In this way, NmPglL contacted each lipid-carrier-linked Pneumococcal CPS peptidoglycan in the presence of rEPA1 or rEPA43, and NmPglL transferred Pneumococcal CPS glycan onto rEPA1 or rEPA43 in vivo:

TABLE 1

| Serotype | Reducing End Structure | | Transfer |
|---|---|---|---|
| Sp8 | 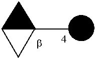 | Glucuronic acid-β1,4- glucose-UndPP | + |
| Sp12F | 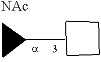 | N-acetyl-fucosamine- α1,3-N-acetyl- galactosamine-UndPP | +++ |
| Sp14 | 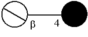 | Galactose-β1,4- glucose-UndPP | + |
| Sp15A | 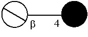 | Galactose-β1,4- glucose-UndPP | − |
| Sp22A | 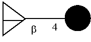 | Rhamnose-β1,4- glucose-UndPP | + |
| Sp23A | 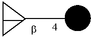 | Rhamnose-β1,4- glucose-UndPP | + |
| Sp33F | 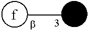 | Galactofuranose-β1,3- glucose-UndPP | ++ |

+) transfer detected
++) good transfer
+++) efficient transfer

The results with respect to Pneumococcal Sp15A CPS were inconclusive because no transfer of Sp15A CPS was detected, but transfer of Pneumococcal Sp14 CPS was detected and both Sp15A and Sp14 CPSes have the reducing end structure Galactose-β1,4-Glucose-UndPP. NmPglL transferred onto rEPA1 and rEPA43 all of the Pneumococcal serotype 8, 12F, 14, 22A, 23A, and 33F glycans (having reducing end structures Glucuronic acid-β1,4-glucose (Sp8), N-acetyl-fucosamine-α1,3-N-acetyl-galactosamine (Sp12F), Galactose-β1,4-glucose (Sp14), Rhamnose-β1,4-glucose (Sp22A, Sp23A), and Galactofuranose-β1,3-glucose (Sp33F), respectively). These results confirm that NmPglL glycan substrates include those having glucose or GalNAc at its reducing end (also supported by Faridmoayer et al. ([3]).

### Example 3

### Identification and Characterization of Neisseria meningitidis PglL Homologues

Twenty Neisseria PglL proteins were identified, each from different Neisseria species. Using established methods, each PglL was first screened for its ability to transfer the S. sonnei O-antigen (made by the operon consisting of the wbgT, wbgU, wzx, wxy, wbgV, wbgW, wbgX, wbgY, and wbgZ genes, encoding proteins of SEQ ID NOs: 208-216, which make a saccharide with a reducing end structure N-acetyl-altruronic acid-α1,3-4-amino-N-acetyl-fucosamine)7 onto an endogenous pilin and with an efficiency that was at least comparable to (i.e., equal to or greater than) that of NmPglL (control). Six Neisseria meningitidis PglL homologues were thereby identified. The six Neisseria PglL proteins were then each screened for its ability to transfer the S. sonnei O-antigen onto rEPA1 and with an efficiency that was at least comparable to NmPglL (control). Four Neisseria meningitidis PglL homologues were thereby identified. For methods see [4], [6], [29], and [31], all incorporated herein by reference in their entireties. The results were as summarized in Table 2 below:

TABLE 2

| | | | RESULTS: | RESULTS: |
|---|---|---|---|---|
| | | | At least comparable | At least comparable |
| | | | transfer of S. sonnei | transfer of S. sonnei |
| | | | O-antigen onto an | O-antigen onto |
| | | Endogenous | endogenous pilin (as | rEPA1 (as |
| | PglL | Pilin | compared to NmPglL | compared to NmPglL |
| | SEQ ID NO: | SEQ ID NO: | transfer onto NmPilE)? | transfer onto rEPA1)? |
| Neisseria meningitidis(control) | 9 | 138 | Control | Control |
| | (NmPglL) | | | |
| Neisseria gonorrhoeae | 11 | 143 | Yes | Yes |
| | (NgPglL) | | | |
| Neisseria lactamica 020-06 | 13 | 148 | Yes | Yes |
| | (NlPglL) | | | |
| Neisseria lactamica ATCC 23970 | 15 | 165 | — | — |
| Neisseria gonorrhoeae F62 | 17 | 166 | — | — |
| Neisseria cinerea ATCC 14685 | 19 | 167 | — | — |
| Neisseria cinerea ATCC 14685 | 19 | 168 | — | — |
| Neisseria mucosa | 21 | 169 | — | — |
| Neisseria mucosa | 21 | 170 | — | — |
| Neisseria flavescens NRL30031/H210 | 23 | 171 | — | — |
| Neisseria mucosa ATCC 25996 | 25 | 172 | Yes | — |
| | (NmuPglL) | | | |
| Neisseria mucosa ATCC 25996 | 25 | 173 | Yes | — |
| | (NmuPglL) | | | |
| Neisseria sp. oral taxon 014 | 27 | 174 | — | — |
| strain F0314 | | | | |
| Neisseria sp. oral taxon 014 | 27 | 175 | — | — |
| strain F0314 | | | | |
| Neisseria arctica | 29 | 176 | — | — |
| Neisseria shayeganii 871 | 31 | 177 | — | — |
| | (Ns2PglL) | | | |
| Neisseria shayeganii 871 | 33 | 177 | Yes | — |
| | (NsPglL) | | | |
| Neisseria shayeganii 871 | 33 | 178 | Yes | — |
| | (NsPglL) | | | |
| Neisseria sp. 83E34 | 35 | 181 | — | — |
| Neisseria sp. 83E34 | 35 | 182 | — | — |
| Neisseria wadsworthii | 37 | 183 | — | — |
| Neisseria wadsworthii | 37 | 184 | — | — |
| Neisseria elongata subsp. glycolytica | 39 | 185 | Yes | Yes |
| ATCC 29315 | (NePglL) | | | |
| Neisseria elongata subsp. glycolytica | 39 | 186 | Yes | Yes |
| ATCC 29315 | (NePglL) | | | |
| Neisseria bacilliformis ATCC | 41 | 187 | Yes | Yes |
| BAA-1200 | (NbPglL) | | | |
| Neisseria bacilliformis ATCC | 41 | 188 | Yes | Yes |
| BAA-1200 | (NbPglL) | | | |
| Neisseria sp. oral taxon 020 str. F0370 | 43 | 190 | — | — |
| Neisseria sp. oral taxon 020 str. F0370 | 43 | 191 | — | — |
| Neisseria sp. 74A18 PglL | 45 | 192 | — | — |
| Neisseria sp. 74A18 PglL | 45 | 193 | — | — |
| Neisseria weaver ATCC 51223 | 47 | 194 | — | — |
| Neisseria macacae ATCC 33926 | 49 | 195 | — | — |
| Neisseria macacae ATCC 33926 | 49 | 196 | — | — |

NmPglL, Neisseria gonorrhoeae PglL (NgPglL), Neisseria lactamica 020-06 (NlPglL), Neisseria elongata subsp. glycolytica ATCC 29315 (NePglL), and Neisseria bacilliformis ATCC BAA-1200 (NbPglL) were shown to transfer the lipid-carrier-linked S. sonnei O-antigen onto the soluble NmPilE-based GlycoTag. For most, this was a glycan transfer onto a non-endogenous GlycoTag. These results indicate that NgPglL, NlPglL, Neisseria mucosa ATCC 25996 (NmuPglL), Neisseria shayeganii 871 (SEQ ID NO: 33) (NsPglL), NePglL, and NbPglL all transfer a lipid linked glycan substrate with a reducing end structure N-acetyl-fucosamine (FucNAc) (S-2 to S-1 structure being N-acetyl-altruronic acid-α1,3-4-amino-N-acetyl-fucosamine) onto its endogenous pilin or rEPA1 with an efficiency that is at least comparable to control (NmPglL). See also Example 8 below.

### Example 4

Designed Carrier Proteins with Internal NmPilE GlycoTag

Twenty-two modified EPA carrier proteins were designed and produced, each incorporating one copy, at an internal residue, of the NmPilE GlycoTag having the sequence SEQ ID NO: 140 (29 amino acid sequence corresponding to residues 45-73 of NmPilE sequence SEQ ID NO: 137). The below-listed EPA residues (numbered with respect to SEQ ID NO: 1) were substituted for the GlycoTag sequence SEQ ID NO: 140 (i.e., an insertion of 29 amino acids):

TABLE 3
Internal EPA residues substituted with GlycoTag SEQ ID NO: 140
(numbered with respect to SEQ ID NO: 1)

| | | |
|---|---|---|
| 1.) | A14 | (rEPA4, SEQ ID NO: 57) |
| 2.) | D36 | (rEPA5, SEQ ID NO: 59) |
| 3.) | Q92 | (rEPA6, SEQ ID NO: 61) |
| 4.) | G123 | (rEPA7, SEQ ID NO: 63) |
| 5.) | E157 | (rEPA8, SEQ ID NO: 65) |
| 6.) | A177 | (rEPA9, SEQ ID NO: 67) |
| 7.) | Y208 | (rEPA10, SEQ ID NO: 69) |
| 8.) | N231 | (rEPA11, SEQ ID NO: 71) |
| 9.) | E252 | (rEPA12, SEQ ID NO: 73) |
| 10.) | R274 | (rEPA13, SEQ ID NO: 75) |
| 11.) | A301 | (rEPA14, SEQ ID NO: 77) |
| 12.) | Q307 | (rEPA15, SEQ ID NO: 79) |
| 13.) | A365 | (rEPA16, SEQ ID NO: 81) |
| 14.) | S408 | (rEPA17, SEQ ID NO: 83) |
| 15.) | T418 | (rEPA18, SEQ ID NO: 85) |
| 16.) | A464 | (rEPA19, SEQ ID NO: 87) |
| 17.) | A519 | (rEPA20, SEQ ID NO: 89) |
| 18.) | G525 | (rEPA21, SEQ ID NO: 91) |
| 19.) | H533 | (rEPA22, SEQ ID NO: 93) |
| 20.) | S585 | (rEPA23, SEQ ID NO: 95) |
| 21.) | K240 | (rEPA24, SEQ ID NO: 97) |
| 22.) | A375 | (rEPA25, SEQ ID NO: 99) |

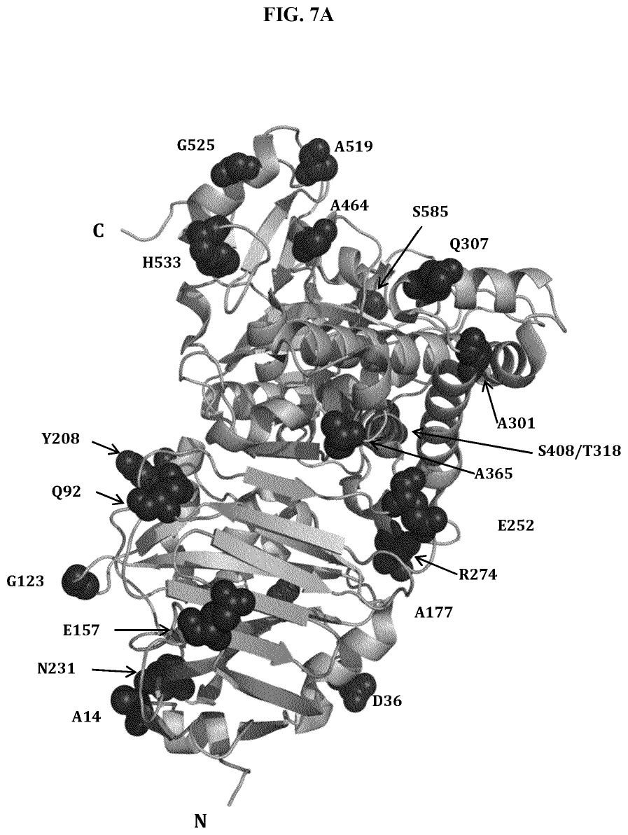

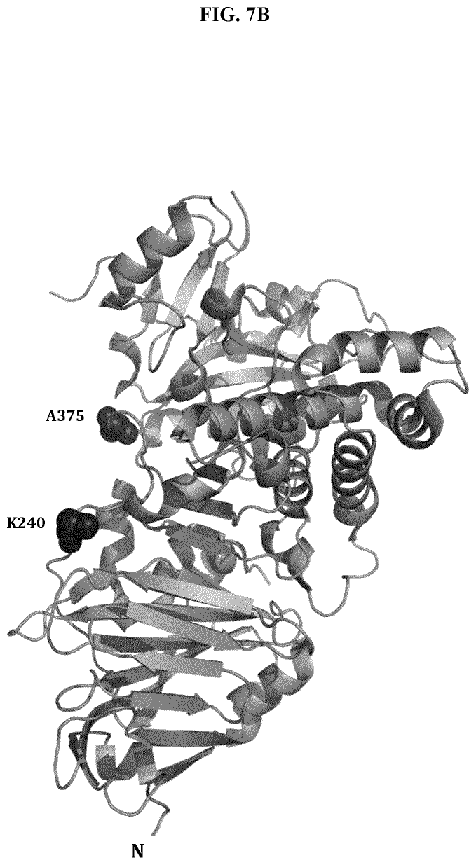

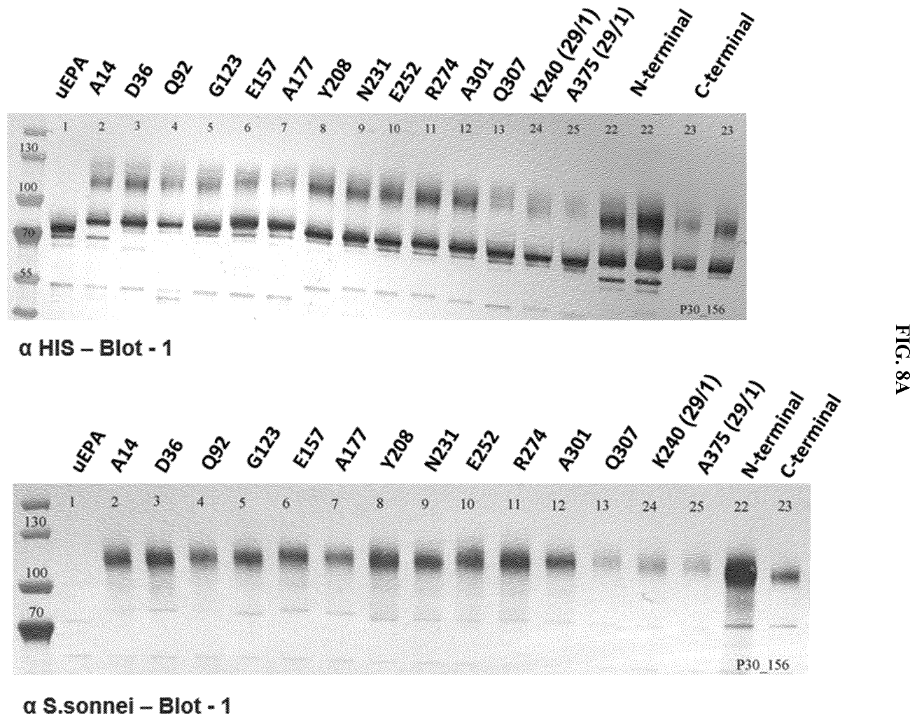

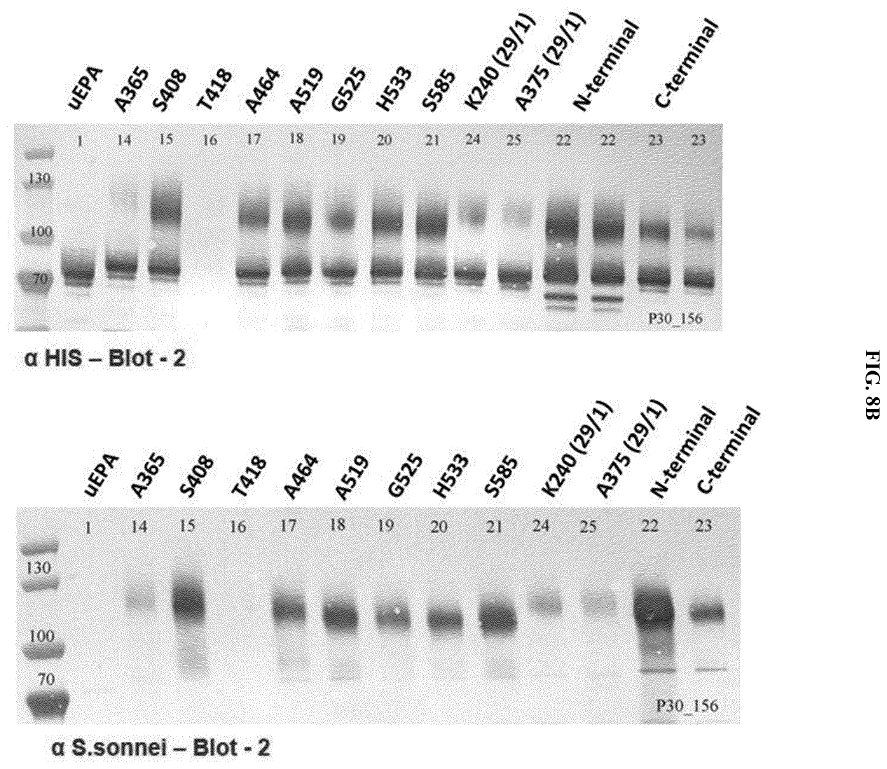

FIG. 7A (depicting EPA residues 1-20.) and FIG. 7B (depicting EPA residues 21.-22.).

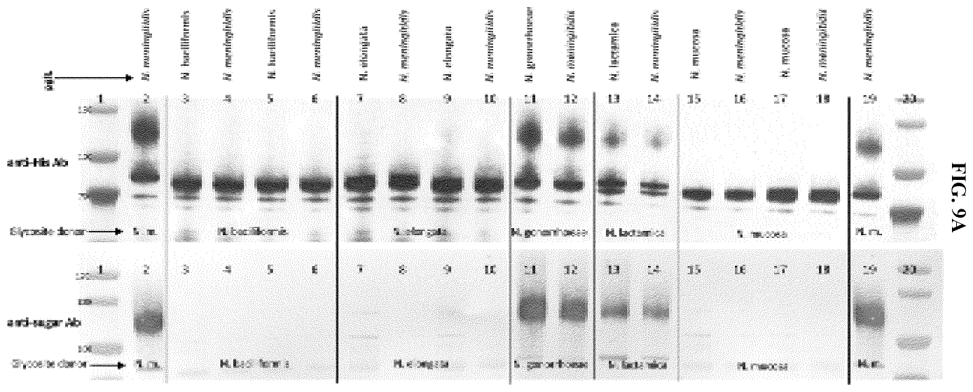

To evaluate glycan transfer to a GlycoTag located within a carrier protein (i.e., an “Internal GlycoTag”), nucleotide sequences encoding NmPglL (SEQ ID NO: 9), S. sonnei O-antigen (SEQ ID NOs: 208-216), and one of each of rEPA4 to rEPA25 (operatively linked to a DsbA periplasmic signal sequence) were introduced into E. coli W3110ΔwaaL (full genotype E. coli W3110 ΔwaaL::pglLNm, ΔwecAwzzECA, ΔO16::wbgT-wbgZ cluster of P. shigelloides O17 Twenty-two different cell lots were made, one for each of rEPA4-rEPA25. Western blot assays confirmed that NmPglL efficiently transferred lipid-carrier-linked S. sonnei O-antigen to all of rEPA4-rEPA17, rEPA19-rEPA25 in vivo. In this experiment, results with respect to rEPA15 were inconclusive because rEPA18 expression was not observed. FIG. 8.

### Example 5

### Homologues of NmPglL Transfer Glycan to Endogenous-Pilin-Based GlycoTag

Homologues of Neisseria meningitidis pilin PilE were identified from Neisseria gonorrhoeae (NgPilin), Neisseria lactamica 020-06 (NiPilin), Neisseria elongate subsp. glycolytica ATCC 29315 (NePilin), and Neisseria bacilliformis ATCC BAA-1200 (NbPilin), Neisseria mucosa ATCC 25996 (NmuPilin), and Neisseria shayeganii 871 (NsPilin) (amino acid sequences SEQ ID NOs 143, 148, 153, 156, 159, and 162, respectively). See also the endogenous pilins in Example 3 and Table 2 above. GlycoTags from each of those pilin were designed.

Using established methods, EPA carrier protein (SEQ ID NO: 1) was modified to incorporate one copy of a GlycoTag from one of each of NgPilin, NlPilin, NePilin, NbPilin, NmuPilin, and NsPilin. Six recombinant EPA (rEPA) proteins were made:
The first EPA having been modified to incorporate, at its N-terminus, the NgPilin GlycoTag SEQ ID NO: 145 (corresponding to residues 52-81 of SEQ ID NO: 143; thirty (30) amino acid long) (rEPA26, SEQ ID NO: 101).
The second EPA having been modified to incorporate, at its N-terminus, the NlPilin GlycoTag SEQ ID NO: 150 (corresponding to residues 52-86 of SEQ ID NO: 148; thirty-five (35) amino acid long) (rEPA27, SEQ ID NO: 103).
The third EPA having been modified to incorporate, at its N-terminus, the NePilin GlycoTag SEQ ID NO: 154 (corresponding to residues 52-96 of SEQ ID NO: 153; fourth-five (45) amino acid long) (rEPA28, SEQ ID NO: 105).
The fourth EPA having been modified to incorporate, at its N-terminus, the NbPilin GlycoTag SEQ ID NO: 157 (corresponding to residues 57-93 of SEQ ID NO: 156; thirty-seven (37) amino acid long) (rEPA29, SEQ ID NO: 107).
The fifth EPA having been modified to incorporate, at its N-terminus, the NmuPilin GlycoTag SEQ ID NO: 160 (corresponding to residues 52-92 of SEQ ID NO: 159; fourty-one (41) amino acid long) (rEPA30, SEQ ID NO: 109).
The sixth EPA having been modified to incorporate, at its N-terminus, the NsPilin GlycoTag SEQ ID NO: 163 (corresponding to residues 53-83 of SEQ ID NO: 162; thirty-one (31) amino acid long) (rEPA31, SEQ ID NO: 111).

Western blot assays was used to determine whether NgPglL (SEQ ID NO: 11), NlPglL (SEQ ID NO: 13), NePglL (SEQ ID NO: 39), NbPglL (SEQ ID NO: 41), NmuPglL (SEQ ID NO: 25) (FIG. 9A) as well as NsPglL (SEQ ID NO: 33) (FIG. 9B) transfer lipid-carrier-linked S. sonnei O-antigen (SEQ ID NOs: 208-216) to carrier proteins containing an endogenous GlycoTag (i.e., to carrier protein rEPA26-rEPA31, respectively). These assays also tested whether NmPglL (SEQ ID NO: 9) can transfer S. sonnei O-antigen onto rEPA26-rEPA31 (FIG. 9A and FIG. 9B).

These results show that PglLs (NgPglL, NlPglL, and NsPglL) transfer a lipid-carrier-linked peptidoglycan having reducing end structure N-acetyl-fucosamine (FucNAc) (S-2 to S-1 structure being N-acetyl-altruronic acid-α1,3-4-amino-N-acetyl-fucosamine) onto a modified EPA carrier protein that has at its N-terminus, an endogenous GlycoTag. Of these three, NgPglL transferred S. sonnei O-antigen to rEPA26 more efficiently than NlPglL transferred S. sonnei O-antigen to rEPA27. Also, NlPglL transferred S. sonnei O-antigen to rEPA27 more efficiently than NsPglL transferred S. sonnei O-antigen to rEPA31. NmPglL also transferred S. sonnei O-antigen onto rEPA26, rEPA27 and rEPA31 FIG. 9A and FIG. 9B.

### Example 6

Designed Carrier Proteins Comprising Neisseria gonnorrhoeae GlycoTag(s)

Modified EPA carrier proteins were designed and produced, each incorporating one or two copies of a Neisseria gonorrhoeae Pilin GlycoTag sequence. Internal EPA residues R274, S408, and/or A519 (numbered with respect to SEQ ID NO: 1) were substituted for the NgPilin GlycoTag having the sequence SEQ ID NO: 145 or SEQ ID NO: 146 (30 amino acid sequence corresponding to residues 52-81 of NgPilin sequence SEQ ID NO: 143 and 20 amino acid sequence corresponding to residues 62-81 of NgPilin sequence SEQ ID NO: 143, respectively) (Table 4 below).

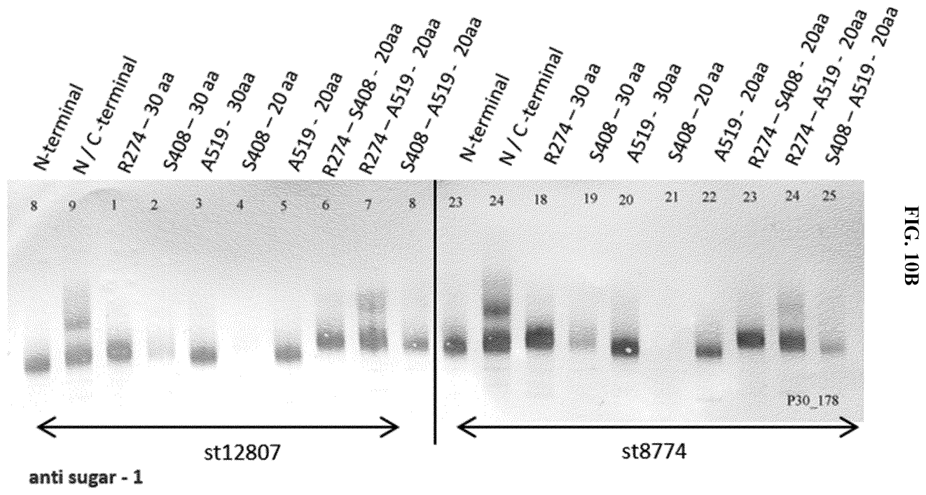

Using established methods, nucleotide sequences encoding Neisseria gonorrhoeae PglL (NgPglL) (SEQ ID NO: 11), Shigella sonnei O-antigen (SEQ ID NOs: 208-216), and one of each of rEPA32-rEPA39 (SEQ ID NOs: 113, 115 117, 119, 121, 123, 125, and 127, respectively, under DsbA periplasmic signal sequence) were operatively introduced into each of two E. coli W3110ΔwaaL host cell strains. Strain “st12807” has the NgPglL sequence integrated at the waaL locus and has genotype: W3110 ΔwaaL, ΔwecAwzzECA ΔO16::wbgT-wbgZ cluster of P. shigelloides O17, ΔwaaL::pglL_Neisseria_gonorrhoeae_CNT56492. Strain “st8774” does not have the NgPglL sequence integrated at the waaL locus and has genotype: W3110 ΔwaaL ΔwecAwzzECA ΔO16::wbgT-wbgZ cluster of P. shigelloides O17. Sixteen cell lots were made, one for each of rEPA32-rEPA39 in each of strains st12807 and st8774. For methods see [4], [6], [29], and [31], all incorporated herein by reference in their entireties. Western blot assays show that NgPglL efficiently transferred lipid-carrier-linked S. sonnei O-antigen to most of the modified EPAs in vivo (rEPA32, rEPA34, rEPA36-rEPA38), but inefficiently for two (rEPA33 and rEPA39), and not at all for one (rEPA35). FIG. 10A, FIG. 10B, and Table 4 below.

TABLE 4

| | | RESULTS: |
|---|---|---|
| | | NgPglL transfer |
| | | lipid-carrier- |
| Modification made to EPA carrier protein | | linked O-antigen |
| (SEQ ID NO: 1) | | onto rEPA#? |
| R274 substituted with SEQ ID | (rEPA32, | Yes, |
| NO: 145 | SEQ ID NO: 113) | efficiently |
| S408 substituted with SEQ ID | (rEPA33, | Yes, but |
| NO: 145 | SEQ ID NO: 115) | inefficiently |
| A519 substituted with SEQ ID | (rEPA34, | Yes, |
| NO: 145 | SEQ ID NO: 117) | efficiently |
| S408 substituted with SEQ ID | (rEPA35, | — |
| NO: 146 | SEQ ID NO: 119) | |
| A519 substituted with SEQ ID | (rEPA36, | Yes, |
| NO: 146 | SEQ ID NO: 121) | efficiently |
| R274 and S408 substituted with | (rEPA37, | Yes, |
| SEQ ID NO: 146 | SEQ ID NO: 123) | efficiently |
| R274 and A519 substituted with | (rEPA38, | Yes, |
| SEQ ID NO: 146 | SEQ ID NO: 125) | efficiently |
| S408 and A519 substituted with | (rEPA39, | Yes, but |
| SEQ ID NO: 146 | SEQ ID NO: 127) | inefficiently |

These results show that a modified EPA carrier protein having internal residues R274, or A519 substituted with either GlycoTag sequence SEQ ID NOs: 145 and 146 is efficient for in vivo O-glycosylation of the modified EPA via NgPglL. Also, a modified EPA carrier protein having both internal residues R274 and A519 substituted with GlycoTag sequence SEQ ID NO 146 is efficient for in vivo O-glycosylation of the modified EPA via NgPglL.

These results also show that a modified EPA carrier protein having internal residue S408 substituted with GlycoTag sequence SEQ ID NO: 145 works, but inefficiently, for in vivo O-glycosylation of the modified EPA via NgPglL This is interesting because, in other studies, a modified EPA carrier protein incorporating a NmGlycoTag at residue S408 was efficiently O-glycosylated by NmPglL (unpublished data).

A modified EPA carrier protein having internal residue S408 substituted with GlycoTag sequence SEQ ID NO: 146 did not work for in vivo O-glycosylation of the modified EPA via NgPglL.

### Example 7

### Systems Comparison

The abilities of NmPglL and NgPglL to transfer glycan to modified EPA carrier proteins comprising a NgPilin GlycoTag were compared. rEPA32, rEPA34, rEPA36, and rEPA38 from Example 6 were used as well as:

Modification made to EPA carrier protein
(SEQ ID NO: 1)

| | |
|---|---|
| SEQ ID NO: 145 introduced at | (rEPA40, |
| the N-terminus | SEQ ID NO: 129) |
| R274 substituted with SEQ ID | (rEPA41, |
| NO: 146 | SEQ ID NO: 131) |
| R274 and A519 substituted with | (rEPA42, |
| SEQ ID NO: 145 | SEQ ID NO: 133) |

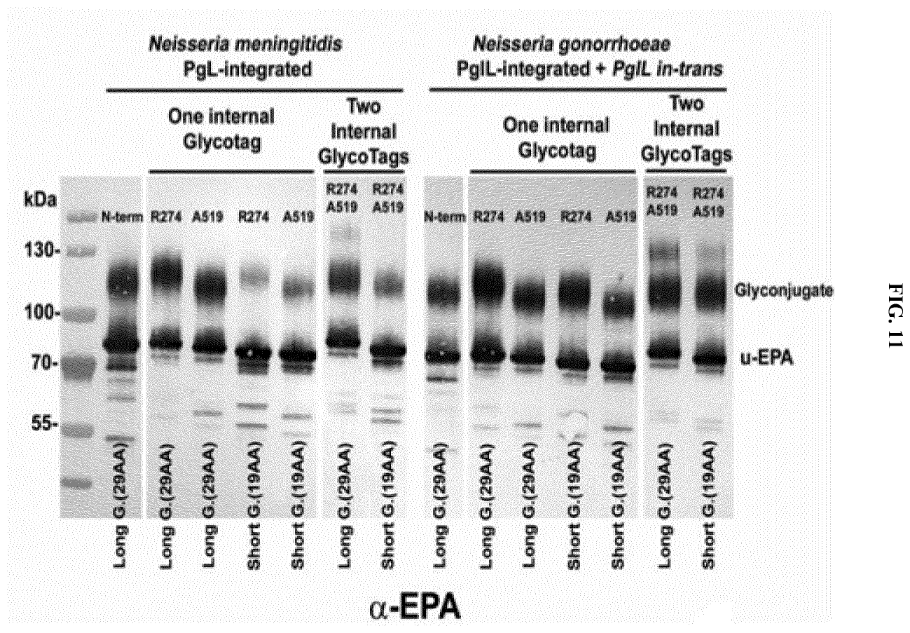

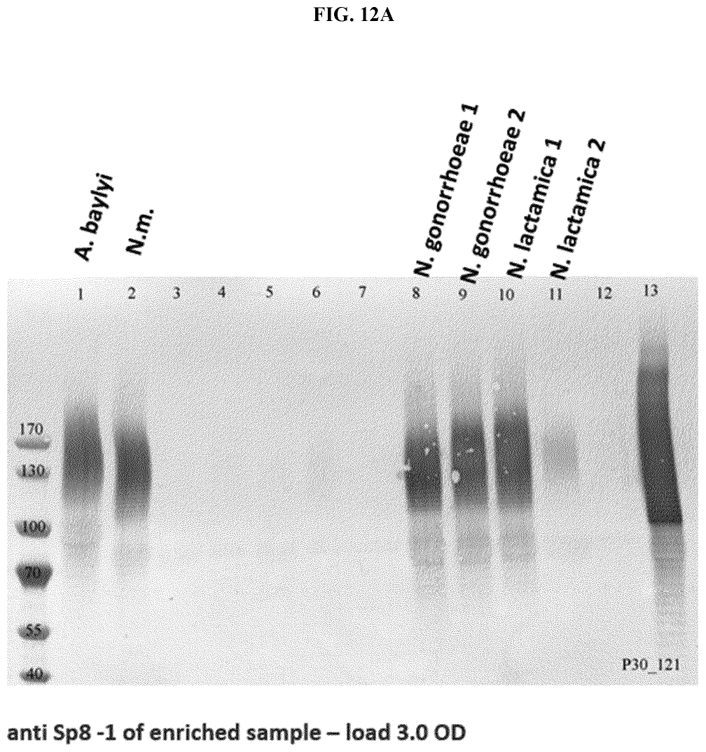

Using established methods, a nucleotide sequence encoding NmPglL (SEQ ID NO: 9) or NgPglL (SEQ ID NO: 11), a nucleotide sequence encoding enzymes required to make Shigella sonnei O-antigen (SEQ ID NOs: 208-216), and a nucleotide sequence encoding one of each of rEPA32, rEPA34, rEPA36, rEPA38, rEPA40, rEPA41, and rEPA42 (under DsbA periplasmic signal sequence) were operatively introduced into E. coli W3110ΔwaaL. Fourteen different cell lots were made. Coomassie blue staining and Western blot assays show that NmPglL efficiently transferred lipid-carrier-linked S. sonnei O-antigen to all of rEPA32, rEPA34, rEPA36, rEPA38, rEPA40, rEPA41, and rEPA42 in vivo. Likewise, NgPglL efficiently transferred lipid-carrier-linked S. sonnei O-antigen to all of rEPA32, rEPA34, rEPA36, rEPA38, rEPA40, rEPA41, and rEPA42 in vivo. FIG. 11.

These results show that NgPilin GlycoTag sequence SEQ ID NO: 145 and NgPilin GlycoTag sequence SEQ ID NO: 146 are efficiently O-glycosylated by both NmPglL and NgPglL when the GlycoTag is introduced at the N-terminus or into an internal residue of a carrier protein, here EPA. This was true when using either one copy or two copies of NgPilin GlycoTag sequences SEQ ID NO: 145 and SEQ ID NO: 146. NgPglL glycosylated GlycoTag sequence SEQ ID NO: 146 more efficiently than did NmPglL. FIG. 11.

### Example 8

NmPglL and NmPglL Homologues Transfer Pneumococcal Capsular Polysaccharides (CPS) to rEPA1

NmPglL and the twenty homologues thereof described in Example 3 were assessed for their ability to transfer Streptococcus pneumoniae serotype Sp8 or Sp22A CPS glycans onto rEPA1 (under DsbA periplasmic signal sequence) in vivo. Pneumococcal Sp8 CPS has a reducing end structure of Glucuronic acid-β1,4-glucose (Table 1). Pneumococcal Sp22A CPS has a reducing end structure of Rhamnose-β1,4-glucose (Table 1).

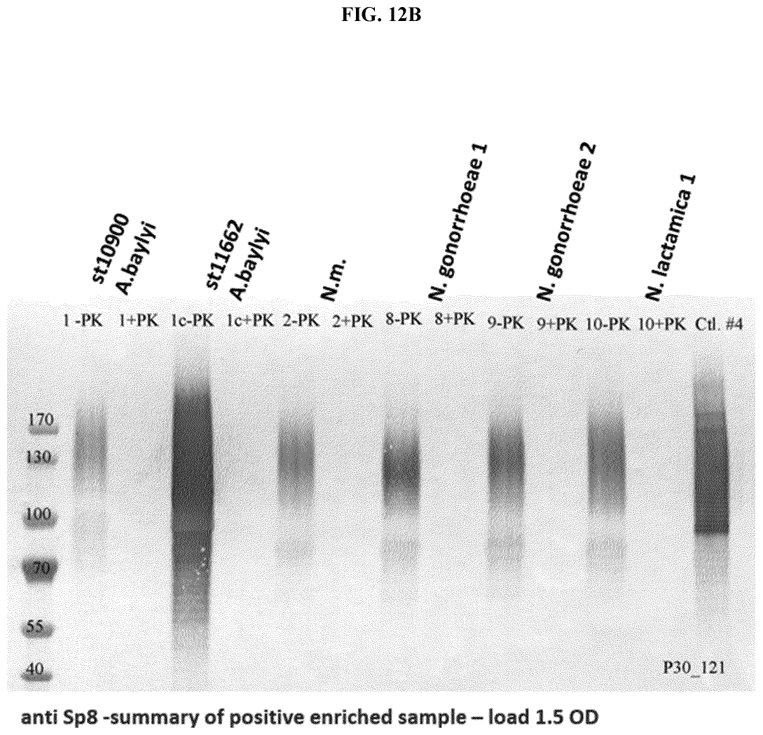

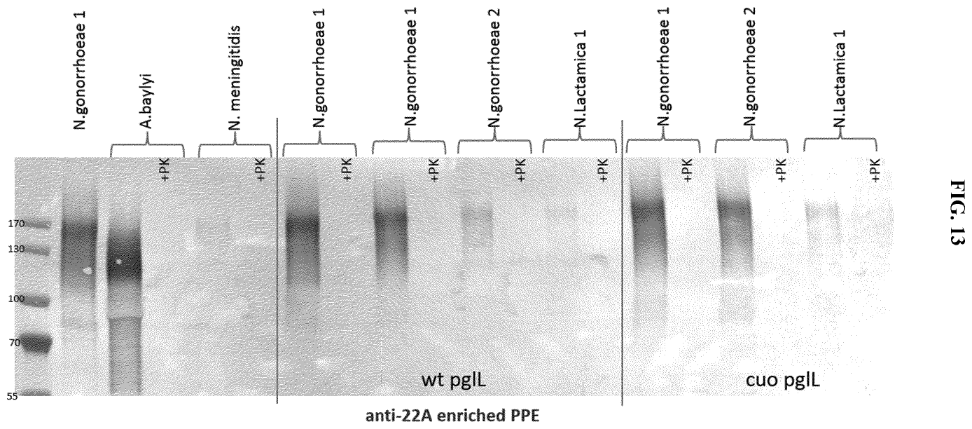

Using established methods, a nucleotide sequence encoding a CPS from Pneumococcal serotype Sp8 or Sp22A, as well as a nucleotide sequence encoding one of the twenty-one Neisserial PglL proteins, and a nucleotide sequence encoding rEPA1 were operatively introduced into E. coli W3110ΔwaaL. Forty-two host cells were made (each CPS being assayed with each of the twenty-one PglLs). In this way, Neisserial PglL contacted each lipid-carrier-linked Pneumococcal CPS peptidoglycan in the presence of rEPA1 and the Neisserial PglL transferred Pneumococcal CPS glycan onto rEPA1 in vivo (Table 5 and FIG. 12A, FIG. 12B, FIG. 13A, and FIG. 13B).

Coomassie blue staining and Western blot assays confirmed that NmPglL, Neisseria gonorrhoeae PglL (NgPglL) (SEQ ID NO: 11), Neisseria lactamica 020-06 (NlPglL) (SEQ ID NO: 13), Neisseria lactamica ATCC 23970 PglL (Nl_{ATCC23970}PglL) (SEQ ID NO: 15), and Neisseria gonorrhoeae F62 PglL (Ng_{F62}PglL) (SEQ ID NO: 17) transfer lipid-carrier-linked Pneomococcal Sp. 8 CPS glycan onto rEPA1. FIG. 12A., FIG. 12B, and Table 5.

Coomassie blue staining and Western blot assays confirmed that NmPglL, NgPglL, NlPglL, and Ng_{F62}PglL transfer lipid-carrier-linked Pneomococcal Sp. 22A CPS glycan onto rEPA1. FIG. 13, and Table 5.

TABLE 5

| | | RESULTS: | RESULTS: |
|---|---|---|---|
| | | Transfer | Transfer |
| | | Pneumococcal | Pneumococcal |
| | | Sp. 8 CPS | Sp. 22A CPS |
| | SEQ | glycan onto | glycan onto |
| | ID NO: | NmPilE? | NmPilE? |
| Neisseria meningitidis PglL | 9 | Yes, | Yes, |
| | | inefficiently | inefficiently |
| Neisseria gonorrhoeae PglL | 11 | Yes | Yes, |
| | | | inefficiently |
| Neisseria lactamica 020-06 PglL | 13 | Yes, | Yes, |
| | | inefficiently | inefficiently |
| Neisseria lactamica ATCC 23970 PglL | 15 | Yes, | — |
| | | inefficiently | |
| Neisseria gonorrhoeae F62 PglL | 17 | Yes | Yes |
| Neisseria cinerea ATCC 14685 PglL | 19 | — | — |
| Neisseria mucosa PglL | 21 | — | — |
| Neisseria flavescens NRL30031/H210 | 23 | — | — |
| PglL | | | |
| Neisseria mucosa ATCC 25996 PglL | 25 | — | — |
| Neisseria sp. oral taxon 014 strain | 27 | — | — |
| F0314 PglL | | | |
| Neisseria arctica PglL | 29 | — | — |
| Neisseria shayeganii 871 PglL | 31 | — | — |
| Neisseria shayeganii 871 PglL | 33 | — | — |
| Neisseria sp. 83E34 PglL | 35 | — | — |
| Neisseria wadsworthii PglL | 37 | — | — |
| Neisseria elongata subsp. glycolytica | 39 | — | — |
| ATCC 29315 PglL | | | |
| Neisseria bacilliformis ATCC | 41 | — | — |
| BAA-1200 PglL | | | |
| Neisseria sp. oral taxon 020 str. | 43 | — | — |
| F0370 PglL | | | |
| Neisseria sp. 74A18 PglL | 45 | — | — |
| Neisseria weaver ATCC 51223 PglL | 47 | — | — |
| Neisseria macacae ATCC 33926 PglL | 49 | — | — |

### Example 9

Comparison of Pilin structures

### GlycoTags:

| | | |
|---|---|---|
| | GlycoTag | GlycoTag |
| Origin | Length | SEQ ID NO: |

GlycoTags 29-35 amino acids in length:

| | | |
|---|---|---|
| Neisseria meningitidis | 29 AAs | 140 |
| Neisseria gonorrhoeae | 30 AAs | 145 |
| Neisseria lactamica 020-06 | 35 AAs | 150 |
| Neisseria shayeganii 871 | 31 AAs | 163 |

GlycoTags 19-20 amino acids in length:

| | | |
|---|---|---|
| Neisseria meningitidis | 19 AAs | 141 |
| Neisseria gonorrhoeae | 20 AAs | 146 |

GlycoTags 12 amino acids in length:

| | | |
|---|---|---|
| Neisseria meningitidis | 12 AAs | 142 |
| Neisseria gonorrhoeae | 12 AAs | 147 |
| Neisseria lactamica 020-06 | 12 AAs | 151 |
| Neisseria shayeganii 871 | 12 AAs | 164 |

SEQ_ID_NO_140
SAVTEYYLNHGEWPGNNTSAGVATS-SEIK------ 29
SEQ_ID_NO_141
----------GEWPGNNTSAGVATS-SEIK------ 19
SEQ_ID_NO_142
----------GEWPGNNTSAGV-------------- 12
SEQ_ID_NO_145
SAVTGYYLNHGTWPKDNTSAGVASSPTDIK------ 30
SEQ_ID_NO_146
----------GTWPKDNTSAGVASSPTDIK------ 20
SEQ_ID_NO_147
----------GTWPKDNTSAGV-------------- 12
SEQ_ID_NO_150
AAVVEYYSDNGTFPAQNASAGIATA-SAITGKYVAK 35
SEQ_ID_NO_151
----------GTFPAQNASAGI-------------- 12
SEQ_ID_NO_163
GAVTEYEADKGVFPTSNASAGVAAA-ADINGK---- 31
SEQ_ID_NO_164
----------GVFPTSNASAGV-------------- 12

### SUMMARY OF SEQUENCES

### Relationship Between Grouped Sequences:

All exemplified modified carrier proteins (rEPA sequences as well as mAcrA, mPcrv, and mCrm197)—SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 199, 202, and 204.
Just rEPA modified carrier protein sequences—SEQ ID NOs: 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, and 135.
Modified carrier proteins which were assayed with NmPglL and S. sonnei O-antigen (see rEPA at Example 1; Acr, Per, and Crm197 at Example 2; and Examples 4, 5, and 7)—SEQ ID NOs: 51, 57, 59, 61, 63, 65, 67, 69, 71, 73, 75, 77, 79, 81, 83, 85, 87, 89, 91, 93, 95, 97, 99, 100, 102, 105, 107, 109, 111, 113, 117, 121, 125, 129, 131, 133, 199, 202, and 204
Modified carrier proteins assayed with NgPglL and S. sonnei O-antigen (see Examples 5, 6, and 7)—SEQ ID NOs: 101, 113, 115, 117, 121, 123, 125, 127, 129, 131, and 133.
   ** sequence features are illustrated (e.g., via underlining) in the specification of the priority application(s)**

### SEQ ID NO: 1

Pseudomonas exotoxin A (EPA) amino acid sequence (mature sequence/signal sequence removed). Corresponds to NCBI Reference Sequence WP_016851883.1.

Pseudomonas exotoxin A (EPA) amino acid sequence
(signal sequence underlined). Corresponds to
NCBI Reference Sequence WP_016851883.1.
SEQ ID NO: 2
MHLIPHWIPLVASLGLLAGGSFASAAEEAFDLWNECAKACVLDLKDGVRS
SRMSVDPAIADTNGQGVLHYSMVLEGGNDALKLAIDNALSITSDGLTIRL
EGGVEPNKPVRYSYTRQARGSWSLNWLVPIGHEKPSNIKVFIHELNAGNQ
LSHMSPIYTIEMGDELLAKLARDATFFVRAHESNEMQPTLAISHAGVSVV
MAQAQPRREKRWSEWASGKVLCLLDPLDGVYNYLAQQRCNLDDTWEGKIY
RVLAGNPAKHDLDIKPTVISHRLHFPEGGSLAALTAHQACHLPLETFTRH
RQPRGWEQLEQCGYPVQRLVALYLAARLSWNQVDQVIRNALASPGSGGDL
GEAIREQPEQARLALTLAAAESERFVRQGTGNDEAGAASADVVSLTCPVA
AGECAGPADSGDALLERNYPTGAEFLGDGGDVSFSTRGTQNWTVERLLQA
HRQLEERGYVFVGYHGTFLEAAQSIVFGGVRARSQDLDAIWRGFYIAGDP
ALAYGYAQDQEPDARGRIRNGALLRVYVPRWSLPGFYRTGLTLAAPEAAG
EVERLIGHPLPLRLDAITGPEEEGGRLETILGWPLAERTVVIPSAIPTDP
RNVGGDLDPSSIPDKEQAISALPDYASQPGKPPREDLK

### SEQ ID NO: 3

Pseudomonas exotoxin A (EPA) polynucleotide sequence. Corresponds to NCBI Accession JX026663.1

### SEQ ID NO: 4

DsbA signal sequence.

### SEQ ID NO: 5

DsbA signal peptide polynucleotide sequence.

### SEQ ID NO: 6

Plesiomonas shigelloides O17 (i.e., Shigella sonnei) O-antigen cluster nucleotide sequence (comprising wbgT, wbgU, wzx, wxy, wbgV, wbgW, wbgX, wbgY, and wbgZ coding regions; 10963 bps). Corresponds to NCBI Genbank Accession AF285970.1. [102]

### SEQ ID NO: 7

PelB signal sequence.

### SEQ ID NO: 8

Neisseria meningitidis PglL (NmPglL) nucleotide sequence.

### SEQ ID NO: 9

Neisseria meningitidis PglL (NmPglL) amino acid sequence. Corresponds to NCBI GenBank Accession AEK98518.1.

### SEQ ID NO: 10

Neisseria gonnorrhoeae PglL (NgPglL) polynucleotide sequence. Corresponds to NCBI GenBank Accession CNT56492.1.

### SEQ ID NO: 11

Neisseria gonnorrhoeae PglL (NgPglL) amino acid sequence. Corresponds to NCBI GenBank Accession CNT56492.1.

### SEQ ID NO: 12

Neisseria lactamica 020-06 PglL (NlPglL) polynucleotide sequence. Corresponds to NCBI GenBank Accession CBN87842.1.

### SEQ ID NO: 13

Neisseria lactamica 020-06 PglL (NlPglL) amino acid sequence. Corresponds to NCBI GenBank Accession CBN87842.1.

### SEQ ID NO: 14

Neisseria lactamica ATCC 23970 PglL (Nl_{ATCC23970}PglL) polynucleotide sequence. Corresponds to NCBI GenBank Accession EEZ75009.1.

### SEQ ID NO: 15

Neisseria lactamica ATCC 23970 PglL (Nl_{ATCC23970}PglL) amino acid sequence. Corresponds to NCBI GenBank Accession EEZ75009.1.

### SEQ ID NO: 16

Neisseria gonorrhoeae F62 PglL (Ng_{F62}PglL) polynucleotide sequence. Corresponds to NCBI GenBank Accession EFF40644.1.

### SEQ ID NO: 17

Neisseria gonorrhoeae F62 PglL (Ng_{F62}PglL) amino acid sequence. Corresponds to NCBI GenBank Accession EFF40644.1.

### SEQ ID NO: 18

Neisseria cinerea ATCC 14685 PglL polynucleotide sequence. Corresponds to NCBI GenBank Accession EEZ72274.1.

### SEQ ID NO: 19

Neisseria cinerea ATCC 14685 PglL amino acid sequence. Corresponds to NCBI GenBank Accession EEZ72274.1.

### SEQ ID NO: 20

Neisseria mucosa PglL polynucleotide sequence. Corresponds to NCBI GenBank Accession KGJ31457.1.

### SEQ ID NO: 21

Neisseria mucosa PglL amino acid sequence. Corresponds to NCBI GenBank Accession KGJ31457.1.

### SEQ ID NO: 22

Neisseria flavescens NRL30031/H210 PglL polynucleotide sequence. Corresponds to NCBI GenBank Accession EEG34481.1.

### SEQ ID NO: 23

Neisseria flavescens NRL30031/H210 PglL amino acid sequence. Corresponds to NCBI GenBank Accession EEG34481.1.

### SEQ ID NO: 24

Neisseria mucosa ATCC 25996 PglL (NmuPglL) polynucleotide seqeuence. Corresponds to NCBI GenBank Accession EFC87884.1.

### SEQ ID NO: 25

Neisseria mucosa ATCC 25996 PglL (NmuPglL) amino acid seqeuence. Corresponds to NCBI GenBank Accession EFC87884.1.

### SEQ ID NO: 26

Neisseria sp. oral taxon 014 strain F0314 PglL polynucleotide sequence. Corresponds to NCBI GenBank Accession EFI23064.1.

### SEQ ID NO: 27

Neisseria sp. oral taxon 014 strain F0314 PglL amino acid sequence. Corresponds to NCBI GenBank Accession EFI23064.1.

### SEQ ID NO: 28

Neisseria arctica PglL polynucleotide sequence. Corresponds to NCBI GenBank Accession KLT72636.1.

### SEQ ID NO: 29

Neisseria arctica PglL amino acid sequence. Corresponds to NCBI GenBank Accession KLT72636.1.

### SEQ ID NO: 30

Neisseria shayeganii 871 PglL (Ns2PglL) polynucleotide sequence. Corresponds to NCBI GenBank Accession EGY51766.1.

### SEQ ID NO: 31

Neisseria shayeganii 871 PglL (Ns2PglL) polynucleotide sequence. Corresponds to NCBI GenBank Accession EGY51766.1.

### SEQ ID NO: 32

Neisseria shayeganii 871 PglL (NsPglL) polynucleotide sequence. Corresponds to NCBI GenBank Accession EGY51593.1.

### SEQ ID NO: 33

Neisseria shayeganii 871 PglL (NsPglL) amino acid sequence. Corresponds to NCBI GenBank Accession EGY51593.1.

### SEQ ID NO: 34

Neisseria sp. 83E34 PglL polynucleotide sequence. Corresponds to NCBI GenBank Accession KPN72282.1.

### SEQ ID NO: 35

Neisseria sp. 83E34 PglL amino acid sequence. Corresponds to NCBI GenBank Accession KPN72282.1.

### SEQ ID NO: 36

Neisseria wadsworthii PglL polynucleotide sequence. Corresponds to NCBI GenBank Accession EGZ44098.1.

### SEQ ID NO: 37

Neisseria wadsworthii PglL amino acid sequence. Corresponds to NCBI GenBank Accession EGZ44098.1.

### SEQ ID NO: 38

Neisseria elongata subsp. glycolytica ATCC 29315 PglL (NePglL) polynucleotide sequence. Corresponds to NCBI GenBank Accession EFE49313.1.

### SEQ ID NO: 39

Neisseria elongata subsp. glycolytica ATCC 29315 PglL (NePglL) amino acid sequence. Corresponds to NCBI GenBank Accession EFE49313.1.

### SEQ ID NO: 40

Neisseria bacilliformis ATCC BAA-1200 PglL (NbPglL) polynucleotide sequence. Corresponds to NCBI GenBank Accession EGF10835.1.

### SEQ ID NO: 41

Neisseria bacilliformis ATCC BAA-1200 PglL (NbPglL) amino acid sequence. Corresponds to NCBI GenBank Accession EGF10835.1.

### SEQ ID NO: 42

Neisseria sp. oral taxon 020 str. F0370 PglL polynucleotide sequence. Corresponds to NCBI GenBank Accession EKY03535.1.

### SEQ ID NO: 43

Neisseria sp. oral taxon 020 str. F0370 PglL amino acid sequence. Corresponds to NCBI GenBank Accession EKY03535.1.

### SEQ ID NO: 44

Neisseria sp. 74A18 PglL polynucleotide sequence. Corresponds to NCBI GenBank Accession KPN74230.1.

### SEQ ID NO: 45

Neisseria sp. 74A18 PglL amino acid sequence. Corresponds to NCBI GenBank Accession KPN74230.1.

### SEQ ID NO: 46

Neisseria weaveri ATCC 51223 PglL polynucleotide sequence. Corresponds to NCBI GenBank Accession EGV35010.1.

### SEQ ID NO: 47

Neisseria weaveri ATCC 51223 PglL amino acid sequence. Corresponds to NCBI GenBank Accession EGV35010.1.

### SEQ ID NO: 48

Neisseria macacae ATCC 33926 PglL polynucleotide sequence. Corresponds to NCBI GenBank Accession EGQ77792.1

### SEQ ID NO: 49

Neisseria macacae ATCC 33926 PglL amino acid sequence. Corresponds to NCBI GenBank Accession EGQ77792.1

### SEQ ID NO: 50

rEPA1 polynucleotide sequence.

rEPA1 amino acid sequence-GlycoTag sequence
SEQ ID NO: 140 at the N-terminus (DsbA signal
sequence underlined, GlycoTag and 6xHis Tag
(SEQ ID NO: 217) double underlined)
SEQ ID NO: 51
MKKIWLALAGLVLAFSASASAVTEYYLNHGEWPGNNTSAGVATSSEIKAE
EAFDLWNECAKACVLDLKDGVRSSRMSVDPAIADTNGQGVLHYSMVLEGG
NDALKLAIDNALSITSDGLTIRLEGGVEPNKPVRYSYTRQARGSWSLNWL
VPIGHEKPSNIKVFIHELNAGNQLSHMSPIYTIEMGDELLAKLARDATFF
VRAHESNEMQPTLAISHAGVSVVMAQAQPRREKRWSEWASGKVLCLLDPL
DGVYNYLAQQRCNLDDTWEGKIYRVLAGNPAKHDLDIKPTVISHRLHFPE
GGSLAALTAHQACHLPLEAFTRHRQPRGWEQLEQCGYPVQRLVALYLAAR
LSWNQVDQVIRNALASPGSGGDLGEAIREQPEQARLALTLAAAESERFVR
QGTGNDEAGAASADVVSLTCPVAAGECAGPADSGDALLERNYPTGAEFLG
DGGDVSFSTRGTQNWTVERLLQAHRQLEERGYVFVGYHGTFLEAAQSIVF
GGVRARSQDLDAIWRGFYIAGDPALAYGYAQDQEPDARGRIRNGALLRVY
VPRWSLPGFYRTGLTLAAPEAAGEVERLIGHPLPLRLDAITGPEEEGGRV
TILGWPLAERTVVIPSAIPTDPRNVGGDLDPSSIPDKEQAISALPDYASQ
PGKPPREDLKHHHHHH

### SEQ ID NO: 52

rEPA2 polynucleotide sequence.

rEPA2 amino acid sequence-GlycoTag sequence
SEQ ID NO: 140 in at residue A375 (DsbA signal
sequence and GlycoTag underlined, 6xHis Tag
(SEQ ID NO: 217) double underlined)
SEQ ID NO: 53
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTRHRQPRGW
EQLEQCGYPVQRLVALYLAARLSWNQVDQVIRNALASPGSGGDLGEAIRE
QPEQARLALTLAAAESERFVRQGTGNDEAGAASADVVSLTCPVASAVTEY
YLNHGEWPGNNTSAGVATSSEIKGECAGPADSGDALLERNYPTGAEFLGD
GGDVSFSTRGTQNWTVERLLQAHRQLEERGYVFVGYHGTFLEAAQSIVFG
GVRARSQDLDAIWRGFYIAGDPALAYGYAQDQEPDARGRIRNGALLRVYV
PRWSLPGFYRTGLTLAAPEAAGEVERLIGHPLPLRLDAITGPEEEGGRVT
ILGWPLAERTVVIPSAIPTDPRNVGGDLDPSSIPDKEQAISALPDYASQP
GKPPREDLKHHHHHH

### SEQ ID NO: 54

rEPA3 polynucleotide sequence.

rEPA3 amino acid sequence-GlycoTag sequence
SEQ ID NO: 140 at C-terminus (DsbA signal
sequence and GlycoTag underlined, 6xHis Tag
(SEQ ID NO: 217) double underlined)
SEQ ID NO: 55
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTRHRQPRGW
EQLEQCGYPVQRLVALYLAARLSWNQVDQVIRNALASPGSGGDLGEAIRE
QPEQARLALTLAAAESERFVRQGTGNDEAGAASADVVSLTCPVAAGECAG
PADSGDALLERNYPTGAEFLGDGGDVSFSTRGTQNWTVERLLQAHRQLEE
RGYVFVGYHGTFLEAAQSIVFGGVRARSQDLDAIWRGFYIAGDPALAYGY
AQDQEPDARGRIRNGALLRVYVPRWSLPGFYRTGLTLAAPEAAGEVERLI
GHPLPLRLDAITGPEEEGGRVTILGWPLAERTVVIPSAIPTDPRNVGGDL
DPSSIPDKEQAISALPDYASQPGKPPREDLKSAVTEYYLNHGEWPGNNTS
AGVATSSEIKHHHHHH

### SEQ ID NO: 56

rEPA4 polynucleotide sequence—GlycoTag sequence SEQ ID NO: 140 in at residue A14.

rEPA4 amino acid sequence - GlycoTag sequence
SEQ ID NO: 140 in at residue A14 (DsbA signal
sequence, GlycoTag underlined, and 6xHis
Tag (SEQ ID NO: 217) underlined).
SEQ ID NO: 57
MKKIWLALAGLVLAFSASAAEEAFDLWNECAKSAVTEYYLNHGEWPGNN
TSAGVATSSEIKCVLDLKDGVRSSRMSVDPAIADTNGQGVLHYSMVLEG
GNDALKLAIDNALSITSDGLTIRLEGGVEPNKPVRYSYTRQARGSWSLN
WLVPIGHEKPSNIKVFIHELNAGNQLSHMSPIYTIEMGDELLAKLARDA
TFFVRAHESNEMQPTLAISHAGVSVVMAQAQPRREKRWSEWASGKVLCL
LDPLDGVYNYLAQQRCNLDDTWEGKIYRVLAGNPAKHDLDIKPTVISHR
LHFPEGGSLAALTAHQACHLPLEAFTRHRQPRGWEQLEQCGYPVQRLVA
LYLAARLSWNQVDQVIRNALASPGSGGDLGEAIREQPEQARLALTLAAA
ESERFVRQGTGNDEAGAASADVVSLTCPVAAGECAGPADSGDALLERNY
PTGAEFLGDGGDVSFSTRGTQNWTVERLLQAHRQLEERGYVFVGYHGTF
LEAAQSIVFGGVRARSQDLDAIWRGFYIAGDPALAYGYAQDQEPDARGR
IRNGALLRVYVPRWSLPGFYRTGLTLAAPEAAGEVERLIGHPLPLRLDA
ITGPEEEGGRVTILGWPLAERTVVIPSAIPTDPRNVGGDLDPSSIPDKE
QAISALPDYASQPGKPPREDLKHHHHHH

### SEQ ID NO: 58

rEPA5 polynucleotide sequence—GlycoTag sequence SEQ ID NO: 140 in at residue D36.

rEPA5 amino acid sequence - GlycoTag sequence
SEQ ID NO: 140 in at residue D36 (DsbA signal
sequence, GlycoTag underlined, and 6xHis
Tag (SEQ ID NO: 217) underlined).
SEQ ID NO: 59
MKKIWLALAGLVLAFSASAAEEAFDLWNECAKACVLDLKDGVRSSRMSV
DPAIASAVTEYYLNHGEWPGNNTSAGVATSSEIKTNGQGVLHYSMVLEG
GNDALKLAIDNALSITSDGLTIRLEGGVEPNKPVRYSYTRQARGSWSLN
WLVPIGHEKPSNIKVFIHELNAGNQLSHMSPIYTIEMGDELLAKLARDA
TFFVRAHESNEMQPTLAISHAGVSVVMAQAQPRREKRWSEWASGKVLCL
LDPLDGVYNYLAQQRCNLDDTWEGKIYRVLAGNPAKHDLDIKPTVISHR
LHFPEGGSLAALTAHQACHLPLEAFTRHRQPRGWEQLEQCGYPVQRLVA
LYLAARLSWNQVDQVIRNALASPGSGGDLGEAIREQPEQARLALTLAAA
ESERFVRQGTGNDEAGAASADVVSLTCPVAAGECAGPADSGDALLERNY
PTGAEFLGDGGDVSFSTRGTQNWTVERLLQAHRQLEERGYVFVGYHGTF
LEAAQSIVFGGVRARSQDLDAIWRGFYIAGDPALAYGYAQDQEPDARGR
IRNGALLRVYVPRWSLPGFYRTGLTLAAPEAAGEVERLIGHPLPLRLDA
ITGPEEEGGRVTILGWPLAERTVVIPSAIPTDPRNVGGDLDPSSIPDKE
QAISALPDYASQPGKPPREDLKHHHHHH

### SEQ ID NO: 60

rEPA6 polynucleotide sequence—GlycoTag sequence SEQ ID NO: 140 in at residue Q92.

rEPA6 amino acid sequence - GlycoTag sequence
SEQ ID NO: 140 in at residue Q92 (DsbA signal
sequence, GlycoTag underlined, and 6xHis
Tag (SEQ ID NO: 217) underlined).
SEQ ID NO: 61
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRSAVTEYYLNHGEWPGNNTSAGVATSSEIKARGSWSLNWLV
PIGHEKPSNIKVFIHELNAGNQLSHMSPIYTIEMGDELLAKLARDATFFV
RAHESNEMQPTLAISHAGVSVVMAQAQPRREKRWSEWASGKVLCLLDPLD
GVYNYLAQQRCNLDDTWEGKIYRVLAGNPAKHDLDIKPTVISHRLHFPEG
GSLAALTAHQACHLPLEAFTRHRQPRGWEQLEQCGYPVQRLVALYLAARL
SWNQVDQVIRNALASPGSGGDLGEAIREQPEQARLALTLAAAESERFVRQ
GTGNDEAGAASADVVSLTCPVAAGECAGPADSGDALLERNYPTGAEFLGD
GGDVSFSTRGTQNWTVERLLQAHRQLEERGYVFVGYHGTFLEAAQSIVFG
GVRARSQDLDAIWRGFYIAGDPALAYGYAQDQEPDARGRIRNGALLRVYV
PRWSLPGFYRTGLTLAAPEAAGEVERLIGHPLPLRLDAITGPEEEGGRVT
ILGWPLAERTVVIPSAIPTDPRNVGGDLDPSSIPDKEQAISALPDYASQP
GKPPREDLKHHHHHH

### SEQ ID NO: 62

rEPA7 polynucleotide sequence—GlycoTag sequence SEQ ID NO: 140 in at residue G123.

rEPA7 amino acid sequence - GlycoTag sequence
SEQ ID NO: 140 in at residue G123 (DsbA signal
sequence, GlycoTag underlined, and 6xHis
Tag (SEQ ID NO: 217) underlined).
SEQ ID NO: 63
MKKIWLALAGLVLAFSASAAEEAFDLWNECAKACVLDLKDGVRSSRMSVDP
AIADTNGQGVLHYSMVLEGGNDALKLAIDNALSITSDGLTIRLEGGVEPNK
PVRYSYTRQARGSWSLNWLVPIGHEKPSNIKVFIHELNASAVTEYYLNHGE
WPGNNTSAGVATSSEIKNQLSHMSPIYTIEMGDELLAKLARDATFFVRAHE
SNEMQPTLAISHAGVSVVMAQAQPRREKRWSEWASGKVLCLLDPLDGVYNY
LAQQRCNLDDTWEGKIYRVLAGNPAKHDLDIKPTVISHRLHFPEGGSLAAL
TAHQACHLPLEAFTRHRQPRGWEQLEQCGYPVQRLVALYLAARLSWNQVDQ
VIRNALASPGSGGDLGEAIREQPEQARLALTLAAAESERFVRQGTGNDEAG
AASADVVSLTCPVAAGECAGPADSGDALLERNYPTGAEFLGDGGDVSFSTR
GTQNWTVERLLQAHRQLEERGYVFVGYHGTFLEAAQSIVFGGVRARSQDLD
AIWRGFYIAGDPALAYGYAQDQEPDARGRIRNGALLRVYVPRWSLPGFYRT
GLTLAAPEAAGEVERLIGHPLPLRLDAITGPEEEGGRVTILGWPLAERTVV
IPSAIPTDPRNVGGDLDPSSIPDKEQAISALPDYASQPGKPPREDLKHHHH
HH

### SEQ ID NO: 64

rEPA8_E157_nucleotide

rEPA8 amino acid sequence - GlycoTag sequence
SEQ ID NO: 140 in at residue E157 (DsbA signal
sequence, GlycoTag underlined, and 6xHis
Tag (SEQ ID NO: 217) underlined).
SEQ ID NO: 65
MKKIWLALAGLVLAFSASAAEEAFDLWNECAKACVLDLKDGVRSSRMSVDP
AIADTNGQGVLHYSMVLEGGNDALKLAIDNALSITSDGLTIRLEGGVEPNK
PVRYSYTRQARGSWSLNWLVPIGHEKPSNIKVFIHELNAGNQLSHMSPIYT
IEMGDELLAKLARDATFFVRAHSAVTEYYLNHGEWPGNNTSAGVATSSEIK
SNEMQPTLAISHAGVSVVMAQAQPRREKRWSEWASGKVLCLLDPLDGVYNY
LAQQRCNLDDTWEGKIYRVLAGNPAKHDLDIKPTVISHRLHFPEGGSLAAL
TAHQACHLPLEAFTRHRQPRGWEQLEQCGYPVQRLVALYLAARLSWNQVDQ
VIRNALASPGSGGDLGEAIREQPEQARLALTLAAAESERFVRQGTGNDEAG
AASADVVSLTCPVAAGECAGPADSGDALLERNYPTGAEFLGDGGDVSFSTR
GTQNWTVERLLQAHRQLEERGYVFVGYHGTFLEAAQSIVFGGVRARSQDLD
AIWRGFYIAGDPALAYGYAQDQEPDARGRIRNGALLRVYVPRWSLPGFYRT
GLTLAAPEAAGEVERLIGHPLPLRLDAITGPEEEGGRVTILGWPLAERTVV
IPSAIPTDPRNVGGDLDPSSIPDKEQAISALPDYASQPGKPPREDLKHHHH
HH

### SEQ ID NO: 66

rEPA9 polynucleotide sequence—GlycoTag sequence SEQ ID NO: 140 in at residue A177.

rEPA9 amino acid sequence - GlycoTag sequence
SEQ ID NO: 140 in at residue A177 (DsbA signal
sequence, GlycoTag underlined, and 6xHis
Tag (SEQ ID NO: 217) underlined).
SEQ ID NO: 67
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMSAVTE
YYLNHGEWPGNNTSAGVATSSEIKQAQPRREKRWSEWASGKVLCLLDPLD
GVYNYLAQQRCNLDDTWEGKIYRVLAGNPAKHDLDIKPTVISHRLHFPEG
GSLAALTAHQACHLPLEAFTRHRQPRGWEQLEQCGYPVQRLVALYLAARL
SWNQVDQVIRNALASPGSGGDLGEAIREQPEQARLALTLAAAESERFVRQ
GTGNDEAGAASADVVSLTCPVAAGECAGPADSGDALLERNYPTGAEFLGD
GGDVSFSTRGTQNWTVERLLQAHRQLEERGYVFVGYHGTFLEAAQSIVFG
GVRARSQDLDAIWRGFYIAGDPALAYGYAQDQEPDARGRIRNGALLRVYV
PRWSLPGFYRTGLTLAAPEAAGEVERLIGHPLPLRLDAITGPEEEGGRVT
ILGWPLAERTVVIPSAIPTDPRNVGGDLDPSSIPDKEQAISALPDYASQP
GKPPREDLKHHHHHH

### SEQ ID NO: 68

rEPA10 polynucleotide sequence—GlycoTag sequence SEQ ID NO: 140 in at residue Y208.

rEPA10 amino acid sequence - GlycoTag sequence
SEQ ID NO: 140 in at residue Y208 (DsbA signal
sequence, GlycoTag underlined, and 6xHis
Tag (SEQ ID NO: 217) underlined).
SEQ ID NO: 69
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNSAVTEYYLNHGEWPGNNTS
AGVATSSEIKLAQQRCNLDDTWEGKIYRVLAGNPAKHDLDIKPTVISHR
LHFPEGGSLAALTAHQACHLPLEAFTRHRQPRGWEQLEQCGYPVQRLVA
LYLAARLSWNQVDQVIRNALASPGSGGDLGEAIREQPEQARLALTLAAA
ESERFVRQGTGNDEAGAASADVVSLTCPVAAGECAGPADSGDALLERNY
PTGAEFLGDGGDVSFSTRGTQNWTVERLLQAHRQLEERGYVFVGYHGTF
LEAAQSIVFGGVRARSQDLDAIWRGFYIAGDPALAYGYAQDQEPDARGR
IRNGALLRVYVPRWSLPGFYRTGLTLAAPEAAGEVERLIGHPLPLRLDA
ITGPEEEGGRVTILGWPLAERTVVIPSAIPTDPRNVGGDLDPSSIPDKE
QAISALPDYASQPGKPPREDLKHHHHHH

### SEQ ID NO: 70

rEPA11 polynucleotide sequence—GlycoTag sequence SEQ ID NO: 140 in at residue N231.

rEPA11 amino acid sequence - GlycoTag sequence
SEQ ID NO: 140 in at residue N231 (DsbA signal
sequence, GlycoTag underlined, and 6xHis
Tag (SEQ ID NO: 217) underlined).
SEQ ID NO: 71
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGSAVTEYYLNHGEWPGNNTSAGVATSSEIKPAKHDLDIKPTVISHR
LHFPEGGSLAALTAHQACHLPLEAFTRHRQPRGWEQLEQCGYPVQRLVA
LYLAARLSWNQVDQVIRNALASPGSGGDLGEAIREQPEQARLALTLAAA
ESERFVRQGTGNDEAGAASADVVSLTCPVAAGECAGPADSGDALLERNY
PTGAEFLGDGGDVSFSTRGTQNWTVERLLQAHRQLEERGYVFVGYHGTF
LEAAQSIVFGGVRARSQDLDAIWRGFYIAGDPALAYGYAQDQEPDARGR
IRNGALLRVYVPRWSLPGFYRTGLTLAAPEAAGEVERLIGHPLPLRLDA
ITGPEEEGGRVTILGWPLAERTVVIPSAIPTDPRNVGGDLDPSSIPDKE
QAISALPDYASQPGKPPREDLKHHHHHH

### SEQ ID NO: 72

rEPA12 polynucleotide sequence—GlycoTag sequence SEQ ID NO: 140 in at residue E252.

rEPA12 amino acid sequence - GlycoTag sequence
SEQ ID NO: 140 in at residue E252 (DsbA signal
sequence, GlycoTag underlined, and 6xHis
Tag (SEQ ID NO: 217) underlined).
SEQ ID NO: 73
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPSAVTEYYLNHGEWPGNNTSAGVATSSEIKG
GSLAALTAHQACHLPLEAFTRHRQPRGWEQLEQCGYPVQRLVALYLAARL
SWNQVDQVIRNALASPGSGGDLGEAIREQPEQARLALTLAAAESERFVRQ
GTGNDEAGAASADVVSLTCPVAAGECAGPADSGDALLERNYPTGAEFLGD
GGDVSFSTRGTQNWTVERLLQAHRQLEERGYVFVGYHGTFLEAAQSIVFG
GVRARSQDLDAIWRGFYIAGDPALAYGYAQDQEPDARGRIRNGALLRVYV
PRWSLPGFYRTGLTLAAPEAAGEVERLIGHPLPLRLDAITGPEEEGGRVT
ILGWPLAERTVVIPSAIPTDPRNVGGDLDPSSIPDKEQAISALPDYASQP
GKPPREDLKHHHHHH

### SEQ ID NO: 74

rEPA13 polynucleotide sequence—GlycoTag sequence SEQ ID NO: 140 in at residue 8274.

rEPA13 amino acid sequence - GlycoTag sequence
SEQ ID NO: 140 in at residue R274 (DsbA signal
sequence, GlycoTag underlined, and 6xHis
Tag (SEQ ID NO: 217) underlined).
SEQ ID NO: 75
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTSAVTEYYL
NHGEWPGNNTSAGVATSSEIKHRQPRGWEQLEQCGYPVQRLVALYLAARL
SWNQVDQVIRNALASPGSGGDLGEAIREQPEQARLALTLAAAESERFVRQ
GTGNDEAGAASADVVSLTCPVAAGECAGPADSGDALLERNYPTGAEFLGD
GGDVSFSTRGTQNWTVERLLQAHRQLEERGYVFVGYHGTFLEAAQSIVFG
GVRARSQDLDAIWRGFYIAGDPALAYGYAQDQEPDARGRIRNGALLRVYV
PRWSLPGFYRTGLTLAAPEAAGEVERLIGHPLPLRLDAITGPEEEGGRVT
ILGWPLAERTVVIPSAIPTDPRNVGGDLDPSSIPDKEQAISALPDYASQP
GKPPREDLKHHHHHH

### SEQ ID NO: 76

rEPA14 polynucleotide sequence—GlycoTag sequence SEQ ID NO: 140 in at residue A301.

rEPA14 amino acid sequence - GlycoTag sequence
SEQ ID NO: 140 in at residue A301 (DsbA signal
sequence, GlycoTag underlined, and 6xHis
Tag (SEQ ID NO: 217) underlined).
SEQ ID NO: 77
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTRHRQPRGW
EQLEQCGYPVQRLVALYLASAVTEYYLNHGEWPGNNTSAGVATSSEIKRL
SWNQVDQVIRNALASPGSGGDLGEAIREQPEQARLALTLAAAESERFVRQ
GTGNDEAGAASADVVSLTCPVAAGECAGPADSGDALLERNYPTGAEFLGD
GGDVSFSTRGTQNWTVERLLQAHRQLEERGYVFVGYHGTFLEAAQSIVFG
GVRARSQDLDAIWRGFYIAGDPALAYGYAQDQEPDARGRIRNGALLRVYV
PRWSLPGFYRTGLTLAAPEAAGEVERLIGHPLPLRLDAITGPEEEGGRVT
ILGWPLAERTVVIPSAIPTDPRNVGGDLDPSSIPDKEQAISALPDYASQP
GKPPREDLKHHHHHH

### SEQ ID NO: 78

rEPA15 polynucleotide sequence—GlycoTag sequence SEQ ID NO: 140 in at residue Q307.

rEPA15 amino acid sequence - GlycoTag sequence
SEQ ID NO: 140 in at residue Q307 (DsbA signal
sequence, GlycoTag underlined, and 6xHis
Tag (SEQ ID NO: 217) underlined).
SEQ ID NO: 79
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLEAFTRH
RQPRGWEQLEQCGYPVQRLVALYLAARLSWNSAVTEYYLNHGEWPGNNT
SAGVATSSEIKVDQVIRNALASPGSGGDLGEAIREQPEQARLALTLAAA
ESERFVRQGTGNDEAGAASADVVSLTCPVAAGECAGPADSGDALLERNY
PTGAEFLGDGGDVSFSTRGTQNWTVERLLQAHRQLEERGYVFVGYHGTF
LEAAQSIVFGGVRARSQDLDAIWRGFYIAGDPALAYGYAQDQEPDARGR
IRNGALLRVYVPRWSLPGFYRTGLTLAAPEAAGEVERLIGHPLPLRLDA
ITGPEEEGGRVTILGWPLAERTVVIPSAIPTDPRNVGGDLDPSSIPDKE
QAISALPDYASQPGKPPREDLKHHHHHH

### SEQ ID NO: 80

rEPA16 polynucleotide sequence—GlycoTag sequence SEQ ID NO: 140 in at residue A365.

rEPA16 amino acid sequence - GlycoTag sequence
SEQ ID NO: 140 in at residue A365 (DsbA signal
sequence, GlycoTag underlined, and 6xHis
Tag (SEQ ID NO: 217) underlined).
SEQ ID NO: 81
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLEAFTRH
RQPRGWEQLEQCGYPVQRLVALYLAARLSWNQVDQVIRNALASPGSGGD
LGEAIREQPEQARLALTLAAAESERFVRQGTGNDEAGAASSAVTEYYLN
HGEWPGNNTSAGVATSSEIKDVVSLTCPVAAGECAGPADSGDALLERNY
PTGAEFLGDGGDVSFSTRGTQNWTVERLLQAHRQLEERGYVFVGYHGTF
LEAAQSIVFGGVRARSQDLDAIWRGFYIAGDPALAYGYAQDQEPDARGR
IRNGALLRVYVPRWSLPGFYRTGLTLAAPEAAGEVERLIGHPLPLRLDA
ITGPEEEGGRVTILGWPLAERTVVIPSAIPTDPRNVGGDLDPSSIPDKE
QAISALPDYASQPGKPPREDLKHHHHHH

### SEQ ID NO: 82

rEPA17 polynucleotide sequence—GlycoTag sequence SEQ ID NO: 140 in at residue S408.

rEPA17 amino acid sequence - GlycoTag sequence
SEQ ID NO: 140 in at residue S408 (DsbA signal
sequence, GlycoTag underlined, and 6xHis
Tag (SEQ ID NO: 217) underlined).
SEQ ID NO: 83
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTRHRQPRGW
EQLEQCGYPVQRLVALYLAARLSWNQVDQVIRNALASPGSGGDLGEAIRE
QPEQARLALTLAAAESERFVRQGTGNDEAGAASADVVSLTCPVAAGECAG
PADSGDALLERNYPTGAEFLGDGGDVSAVTEYYLNHGEWPGNNTSAGVAT
SSEIKFSTRGTQNWTVERLLQAHRQLEERGYVFVGYHGTFLEAAQSIVFG
GVRARSQDLDAIWRGFYIAGDPALAYGYAQDQEPDARGRIRNGALLRVYV
PRWSLPGFYRTGLTLAAPEAAGEVERLIGHPLPLRLDAITGPEEEGGRVT
ILGWPLAERTVVIPSAIPTDPRNVGGDLDPSSIPDKEQAISALPDYASQP
GKPPREDLKHHHHHH

### SEQ ID NO: 84

rEPA18 polynucleotide sequence—GlycoTag sequence SEQ ID NO: 140 in at residue T418.

rEPA18 amino acid sequence - GlycoTag sequence
SEQ ID NO: 140 in at residue T418 (DsbA signal
sequence, GlycoTag underlined, and 6xHis
Tag (SEQ ID NO: 217) underlined).
SEQ ID NO: 85
MKKIWLALAGLVLAFSASAAEEAFDLWNECAKACVLDLKDGVRSSRMS
VDPAIADTNGQGVLHYSMVLEGGNDALKLAIDNALSITSDGLTIRLEG
GVEPNKPVRYSYTRQARGSWSLNWLVPIGHEKPSNIKVFIHELNAGNQ
LSHMSPIYTIEMGDELLAKLARDATFFVRAHESNEMQPTLAISHAGVS
VVMAQAQPRREKRWSEWASGKVLCLLDPLDGVYNYLAQQRCNLDDTWE
GKIYRVLAGNPAKHDLDIKPTVISHRLHFPEGGSLAALTAHQACHLPL
EAFTRHRQPRGWEQLEQCGYPVQRLVALYLAARLSWNQVDQVIRNALA
SPGSGGDLGEAIREQPEQARLALTLAAAESERFVRQGTGNDEAGAASA
DVVSLTCPVAAGECAGPADSGDALLERNYPTGAEFLGDGGDVSFSTRG
TQNWSAVTEYYLNHGEWPGNNTSAGVATSSEIKVERLLQAHRQLEERG
YVFVGYHGTFLEAAQSIVFGGVRARSQDLDAIWRGFYIAGDPALAYGY
AQDQEPDARGRIRNGALLRVYVPRWSLPGFYRTGLTLAAPEAAGEVER
LIGHPLPLRLDAITGPEEEGGRVTILGWPLAERTVVIPSAIPTDPRNV
GGDLDPSSIPDKEQAISALPDYASQPGKPPREDLKHHHHHH

### SEQ ID NO: 86

rEPA19 polynucleotide sequence—GlycoTag sequence SEQ ID NO: 140 in at residue A464.

rEPA19 amino acid sequence-GlycoTag sequence
SEQ ID NO: 140 in at residue A464 (DsbA signal
sequence, GlycoTag underlined, and 6xHis
Tag (SEQ ID NO: 217) underlined).
SEQ ID NO: 87
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTRHRQPRGWEQLEQC
GYPVQRLVALYLAARLSWNQVDQVIRNALASPGS
GGDLGEAIREQPEQARLALTLAAAESERFVRQGT
GNDEAGAASADVVSLTCPVAAGECAGPADSGDAL
LERNYPTGAEFLGDGGDVSFSTRGTQNWTVERLL
QAHRQLEERGYVFVGYHGTFLEAAQSIVFGGVRA
RSQDLDSAVTEYYLNHGEWPGNNTSAGVATSSEI
KIWRGFYIAGDPALAYGYAQDQEPDARGRIRNGA
LLRVYVPRWSLPGFYRTGLTLAAPEAAGEVERLI
GHPLPLRLDAITGPEEEGGRVTILGWPLAERTVV
IPSAIPTDPRNVGGDLDPSSIPDKEQAISALPDY
ASQPGKPPREDLKHHHHHH

### SEQ ID NO: 88

rEPA20 polynucleotide sequence—GlycoTag sequence SEQ ID NO: 140 in at residue A519.

rEPA20 amino acid sequence-GlycoTag sequence
SEQ ID NO: 140 in at residue A519 (DsbA signal
sequence, GlycoTag underlined, and 6xHis Tag
(SEQ ID NO: 217) underlined).
SEQ ID NO: 89
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTRHRQPRGWEQLEQC
GYPVQRLVALYLAARLSWNQVDQVIRNALASPGS
GGDLGEAIREQPEQARLALTLAAAESERFVRQGT
GNDEAGAASADVVSLTCPVAAGECAGPADSGDAL
LERNYPTGAEFLGDGGDVSFSTRGTQNWTVERLL
QAHRQLEERGYVFVGYHGTFLEAAQSIVFGGVRA
RSQDLDAIWRGFYIAGDPALAYGYAQDQEPDARG
RIRNGALLRVYVPRWSLPGFYRTGLTLSAVTEYY
LNHGEWPGNNTSAGVATSSEIKAPEAAGEVERLI
GHPLPLRLDAITGPEEEGGRVTILGWPLAERTVV
IPSAIPTDPRNVGGDLDPSSIPDKEQAISALPDY
ASQPGKPPREDLKHHHHHH

### SEQ ID NO: 90

rEPA21 polynucleotide sequence—GlycoTag sequence SEQ ID NO: 140 in at residue G525.

rEPA21 amino acid sequence-GlycoTag sequence
SEQ ID NO: 140 in at residue G525 (DsbA signal
sequence, GlycoTag underlined, and 6xHis Tag
(SEQ ID NO: 217) underlined).
SEQ ID NO: 91
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTRHRQPRGWEQLEQC
GYPVQRLVALYLAARLSWNQVDQVIRNALASPGS
GGDLGEAIREQPEQARLALTLAAAESERFVRQGT
GNDEAGAASADVVSLTCPVAAGECAGPADSGDAL
LERNYPTGAEFLGDGGDVSFSTRGTQNWTVERLL
QAHRQLEERGYVFVGYHGTFLEAAQSIVFGGVRA
RSQDLDAIWRGFYIAGDPALAYGYAQDQEPDARG
RIRNGALLRVYVPRWSLPGFYRTGLTLAAPEAAS
AVTEYYLNHGEWPGNNTSAGVATSSEIKEVERLI
GHPLPLRLDAITGPEEEGGRVTILGWPLAERTVV
IPSAIPTDPRNVGGDLDPSSIPDKEQAISALPDY
ASQPGKPPREDLKHHHHHH

### SEQ ID NO: 92

rEPA22 polynucleotide sequence—GlycoTag sequence SEQ ID NO: 140 in at residue H533.

rEPA22 amino acid sequence-GlycoTag sequence
SEQ ID NO: 140 in at residue H533 (DsbA signal
sequence, GlycoTag underlined, and 6xHis Tag
(SEQ ID NO: 217) underlined).
SEQ ID NO: 93
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTRHRQPRGWEQLEQC
GYPVQRLVALYLAARLSWNQVDQVIRNALASPGS
GGDLGEAIREQPEQARLALTLAAAESERFVRQGT
GNDEAGAASADVVSLTCPVAAGECAGPADSGDAL
LERNYPTGAEFLGDGGDVSFSTRGTQNWTVERLL
QAHRQLEERGYVFVGYHGTFLEAAQSIVFGGVRA
RSQDLDAIWRGFYIAGDPALAYGYAQDQEPDARG
RIRNGALLRVYVPRWSLPGFYRTGLTLAAPEAAG
EVERLIGSAVTEYYLNHGEWPGNNTSAGVATSSE
IKPLPLRLDAITGPEEEGGRVTILGWPLAERTVV
IPSAIPTDPRNVGGDLDPSSIPDKEQAISALPDY
ASQPGKPPREDLKHHHHHH

### SEQ ID NO: 94

rEPA23 polynucleotide sequence—GlycoTag sequence SEQ ID NO: 140 in at residue S585.

rEPA23 amino acid sequence-GlycoTag sequence
SEQ ID NO: 140 in at residue S585 (DsbA signal
sequence, GlycoTag underlined, and 6xHis Tag
(SEQ ID NO: 217) underlined).
SEQ ID NO: 95
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTRHRQPRGWEQLEQC
GYPVQRLVALYLAARLSWNQVDQVIRNALASPGS
GGDLGEAIREQPEQARLALTLAAAESERFVRQGT
GNDEAGAASADVVSLTCPVAAGECAGPADSGDAL
LERNYPTGAEFLGDGGDVSFSTRGTQNWTVERLL
QAHRQLEERGYVFVGYHGTFLEAAQSIVFGGVRA
RSQDLDAIWRGFYIAGDPALAYGYAQDQEPDARG
RIRNGALLRVYVPRWSLPGFYRTGLTLAAPEAAG
EVERLIGHPLPLRLDAITGPEEEGGRVTILGWPL
AERTVVIPSAIPTDPRNVGGDLDPSSAVTEYYLN
HGEWPGNNTSAGVATSSEIKIPDKEQAISALPDY
ASQPGKPPREDLKHHHHHH

### SEQ ID NO: 96

rEPA24_polynucleotide sequence—GlycoTag sequence SEQ ID NO: 140 in at residue K240.

rEPA24_amino acid sequence-GlycoTag sequence
SEQ ID NO: 140 in at residue K240 (DsbA signal
sequence, GlycoTag underlined, and 6xHis Tag
(SEQ ID NO: 217) underlined).
SEQ ID NO: 97
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDISAVTEYYLNHGEWP
GNNTSAGVATSSEIKPTVISHRLHFPEGGSLAAL
TAHQACHLPLEAFTRHRQPRGWEQLEQCGYPVQR
LVALYLAARLSWNQVDQVIRNALASPGSGGDLGE
AIREQPEQARLALTLAAAESERFVRQGTGNDEAG
AASADVVSLTCPVAAGECAGPADSGDALLERNYP
TGAEFLGDGGDVSFSTRGTQNWTVERLLQAHRQL
EERGYVFVGYHGTFLEAAQSIVFGGVRARSQDLD
AIWRGFYIAGDPALAYGYAQDQEPDARGRIRNGA
LLRVYVPRWSLPGFYRTGLTLAAPEAAGEVERLI
GHPLPLRLDAITGPEEEGGRVTILGWPLAERTVV
IPSAIPTDPRNVGGDLDPSSIPDKEQAISALPDY
ASQPGKPPREDLKHHHHHH

### SEQ ID NO: 98

rEPA25 polynucleotide sequence—GlycoTag sequence SEQ ID NO: 140 in at residue A375.

rEPA25 amino acid sequence-GlycoTag sequence
SEQ ID NO: 140 in at residue A375 (DsbA signal
sequence, GlycoTag underlined, and 6xHis Tag
(SEQ ID NO: 217) underlined).
SEQ ID NO: 99
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTRHRQPRGWEQLEQC
GYPVQRLVALYLAARLSWNQVDQVIRNALASPGS
GGDLGEAIREQPEQARLALTLAAAESERFVRQGT
GNDEAGAASADVVSLTCPVASAVTEYYLNHGEWP
GNNTSAGVATSSEIKGECAGPADSGDALLERNYP
TGAEFLGDGGDVSFSTRGTQNWTVERLLQAHRQL
EERGYVFVGYHGTFLEAAQSIVFGGVRARSQDLD
AIWRGFYIAGDPALAYGYAQDQEPDARGRIRNGA
LLRVYVPRWSLPGFYRTGLTLAAPEAAGEVERLI
GHPLPLRLDAITGPEEEGGRVTILGWPLAERTVV
IPSAIPTDPRNVGGDLDPSSIPDKEQAISALPDY
ASQPGKPPREDLKHHHHHH

### SEQ ID NO: 100

rEPA26 polynucleotide sequence—GlycotTag sequence SEQ ID NO: 145 at N-terminus.

rEPA26 amino acid sequence-GlycoTag sequence
SEQ ID NO: 145 at N-terminus (DsbA signal
sequence and 6xHis Tag (SEQ ID NO: 217)
underlined, GlycoTag double underlined).
SEQ ID NO: 101
MKKIWLALAGLVLAFSASASSAVTGYYLNHGTWP
KDNTSAGVASSPTDIKAEEAFDLWNECAKACVLD
LKDGVRSSRMSVDPAIADTNGQGVLHYSMVLEGG
NDALKLAIDNALSITSDGLTIRLEGGVEPNKPVR
YSYTRQARGSWSLNWLVPIGHEKPSNIKVFIHEL
NAGNQLSHMSPIYTIEMGDELLAKLARDATFFVR
AHESNEMQPTLAISHAGVSVVMAQAQPRREKRWS
EWASGKVLCLLDPLDGVYNYLAQQRCNLDDTWEG
KIYRVLAGNPAKHDLDIKPTVISHRLHFPEGGSL
AALTAHQACHLPLEAFTRHRQPRGWEQLEQCGYP
VQRLVALYLAARLSWNQVDQVIRNALASPGSGGD
LGEAIREQPEQARLALTLAAAESERFVRQGTGND
EAGAASADVVSLTCPVAAGECAGPADSGDALLER
NYPTGAEFLGDGGDVSFSTRGTQNWTVERLLQAH
RQLEERGYVFVGYHGTFLEAAQSIVFGGVRARSQ
DLDAIWRGFYIAGDPALAYGYAQDQEPDARGRIR
NGALLRVYVPRWSLPGFYRTGLTLAAPEAAGEVE
RLIGHPLPLRLDAITGPEEEGGRVTILGWPLAER
TVVIPSAIPTDPRNVGGDLDPSSIPDKEQAISAL
PDYASQPGKPPREDLKHHHHHH

### SEQ ID NO: 102

rEPA27 polynucleotide sequence—GlycoTag sequence SEQ ID NO: 150 at N-terminus.

rEPA27 amino acid sequence-GlycoTag sequence
SEQ ID NO: 150 at N-terminus (DsbA signal
sequence and 6xHis Tag (SEQ ID NO: 217)
underlined, GlycoTag double underlined).
SEQ ID NO: 103
MKKIWLALAGLVLAFSASASAAVVEYYSDNGTFP
AQNASAGIATASAITGKYVAKAEEAFDLWNECAK
ACVLDLKDGVRSSRMSVDPAIADTNGQGVLHYSM
VLEGGNDALKLAIDNALSITSDGLTIRLEGGVEP
NKPVRYSYTRQARGSWSLNWLVPIGHEKPSNIKV
FIHELNAGNQLSHMSPIYTIEMGDELLAKLARDA
TFFVRAHESNEMQPTLAISHAGVSVVMAQAQPRR
EKRWSEWASGKVLCLLDPLDGVYNYLAQQRCNLD
DTWEGKIYRVLAGNPAKHDLDIKPTVISHRLHFP
EGGSLAALTAHQACHLPLEAFTRHRQPRGWEQLE
QCGYPVQRLVALYLAARLSWNQVDQVIRNALASP
GSGGDLGEAIREQPEQARLALTLAAAESERFVRQ
GTGNDEAGAASADVVSLTCPVAAGECAGPADSGD
ALLERNYPTGAEFLGDGGDVSFSTRGTQNWTVER
LLQAHRQLEERGYVFVGYHGTFLEAAQSIVFGGV
RARSQDLDAIWRGFYIAGDPALAYGYAQDQEPDA
RGRIRNGALLRVYVPRWSLPGFYRTGLTLAAPEA
AGEVERLIGHPLPLRLDAITGPEEEGGRVTILGW
PLAERTVVIPSAIPTDPRNVGGDLDPSSIPDKEQ
AISALPDYASQPGKPPREDLKHHHHHH

### SEQ ID NO: 104

rEPA28 polynucleotide sequence—GlycoTag sequence SEQ ID NO: 154 at N-terminus.

rEPA28 amino acid sequence-GlycoTag sequence
SEQ ID NO: 154 at N-terminus (DsbA signal
sequence and 6xHis Tag (SEQ ID NO: 217)
underlined, GlycoTag double underlined).
SEQ ID NO: 105
MKKIWLALAGLVLAFSASASSALSEAFQTDGITG
MTAAAKAFNKTAAAGGGAGGAAAAGTQHASKAEE
AFDLWNECAKACVLDLKDGVRSSRMSVDPAIADT
NGQGVLHYSMVLEGGNDALKLAIDNALSITSDGL
TIRLEGGVEPNKPVRYSYTRQARGSWSLNWLVPI
GHEKPSNIKVFIHELNAGNQLSHMSPIYTIEMGD
ELLAKLARDATFFVRAHESNEMQPTLAISHAGVS
VVMAQAQPRREKRWSEWASGKVLCLLDPLDGVYN
YLAQQRCNLDDTWEGKIYRVLAGNPAKHDLDIKP
TVISHRLHFPEGGSLAALTAHQACHLPLEAFTRH
RQPRGWEQLEQCGYPVQRLVALYLAARLSWNQVD
QVIRNALASPGSGGDLGEAIREQPEQARLALTLA
AAESERFVRQGTGNDEAGAASADVVSLTCPVAAG
ECAGPADSGDALLERNYPTGAEFLGDGGDVSFST
RGTQNWTVERLLQAHRQLEERGYVFVGYHGTFLE
AAQSIVFGGVRARSQDLDAIWRGFYIAGDPALAY
GYAQDQEPDARGRIRNGALLRVYVPRWSLPGFYR
TGLTLAAPEAAGEVERLIGHPLPLRLDAITGPEE
EGGRVTILGWPLAERTVVIPSAIPTDPRNVGGDL
DPSSIPDKEQAISALPDYASQPGKPPREDLKHHH
HHH

### SEQ ID NO: 106

rEPA29 polynucleotide sequence—GlycoTag sequence SEQ ID NO: 157 at N-terminus.

rEPA29 amino acid sequence-GlycoTag sequence
SEQ ID NO: 157 at N-terminus (DsbA signal
sequence and 6xHis Tag (SEQ ID NO: 217)
underlined, GlycoTag double underlined).
SEQ ID NO: 107
MKKIWLALAGLVLAFSASASTLISTDATSINDLD
IAVAAWNRQANNTGANSKYVTSVAEEAFDLWNEC
AKACVLDLKDGVRSSRMSVDPAIADTNGQGVLHY
SMVLEGGNDALKLAIDNALSITSDGLTIRLEGGV
EPNKPVRYSYTRQARGSWSLNWLVPIGHEKPSNI
KVFIHELNAGNQLSHMSPIYTIEMGDELLAKLAR
DATFFVRAHESNEMQPTLAISHAGVSVVMAQAQP
RREKRWSEWASGKVLCLLDPLDGVYNYLAQQRCN
LDDTWEGKIYRVLAGNPAKHDLDIKPTVISHRLH
FPEGGSLAALTAHQACHLPLEAFTRHRQPRGWEQ
LEQCGYPVQRLVALYLAARLSWNQVDQVIRNALA
SPGSGGDLGEAIREQPEQARLALTLAAAESERFV
RQGTGNDEAGAASADVVSLTCPVAAGECAGPADS
GDALLERNYPTGAEFLGDGGDVSFSTRGTQNWTV
ERLLQAHRQLEERGYVFVGYHGTFLEAAQSIVFG
GVRARSQDLDAIWRGFYIAGDPALAYGYAQDQEP
DARGRIRNGALLRVYVPRWSLPGFYRTGLTLAAP
EAAGEVERLIGHPLPLRLDAITGPEEEGGRVTIL
GWPLAERTVVIPSAIPTDPRNVGGDLDPSSIPDK
EQAISALPDYASQPGKPPREDLKHHHHHH

### SEQ ID NO: 108

rEPA30 polynucleotide sequence—GlycoTag sequence SEQ ID NO: 160 at N-terminus.

rEPA30 amino acid sequence-GlycoTag sequence
SEQ ID NO: 160 at N-terminus (DsbA signal
sequence and 6xHis Tag (SEQ ID NO: 217)
underlined, GlycoTag double underlined).
SEQ ID NO: 109
MKKIWLALAGLVLAFSASASTPLVEAVAASSNAI
ACKNNAPWYTSSVQSGKYVSAIEPAVKAEEAFDL
WNECAKACVLDLKDGVRSSRMSVDPAIADTNGQG
VLHYSMVLEGGNDALKLAIDNALSITSDGLTIRL
EGGVEPNKPVRYSYTRQARGSWSLNWLVPIGHEK
PSNIKVFIHELNAGNQLSHMSPIYTIEMGDELLA
KLARDATFFVRAHESNEMQPTLAISHAGVSVVMA
QAQPRREKRWSEWASGKVLCLLDPLDGVYNYLAQ
QRCNLDDTWEGKIYRVLAGNPAKHDLDIKPTVIS
HRLHFPEGGSLAALTAHQACHLPLEAFTRHRQPR
GWEQLEQCGYPVQRLVALYLAARLSWNQVDQVIR
NALASPGSGGDLGEAIREQPEQARLALTLAAAES
ERFVRQGTGNDEAGAASADVVSLTCPVAAGECAG
PADSGDALLERNYPTGAEFLGDGGDVSFSTRGTQ
NWTVERLLQAHRQLEERGYVFVGYHGTFLEAAQS
IVFGGVRARSQDLDAIWRGFYIAGDPALAYGYAQ
DQEPDARGRIRNGALLRVYVPRWSLPGFYRTGLT
LAAPEAAGEVERLIGHPLPLRLDAITGPEEEGGR
VTILGWPLAERTVVIPSAIPTDPRNVGGDLDPSS
IPDKEQAISALPDYASQPGKPPREDLKHHHHHH

### SEQ ID NO: 110

rEPA31 polynucleotide sequence—GlycoTag sequence SEQ ID NO: 163 at N-terminus.

rEPA31_amino acid sequence-GlycoTag sequence
SEQ ID NO: 163 at N-terminus (DsbA signal
sequence and 6xHis Tag (SEQ ID NO: 217)
underlined, GlycoTag double underlined).
SEQ ID NO: 111
MKKIWLALAGLVLAFSASASGAVTEYEADKGVFP
TSNASAGVAAAADINGKAEEAFDLWNECAKACVL
DLKDGVRSSRMSVDPAIADTNGQGVLHYSMVLEG
GNDALKLAIDNALSITSDGLTIRLEGGVEPNKPV
RYSYTRQARGSWSLNWLVPIGHEKPSNIKVFIHE
LNAGNQLSHMSPIYTIEMGDELLAKLARDATFFV
RAHESNEMQPTLAISHAGVSVVMAQAQPRREKRW
SEWASGKVLCLLDPLDGVYNYLAQQRCNLDDTWE
GKIYRVLAGNPAKHDLDIKPTVISHRLHFPEGGS
LAALTAHQACHLPLEAFTRHRQPRGWEQLEQCGY
PVQRLVALYLAARLSWNQVDQVIRNALASPGSGG
DLGEAIREQPEQARLALTLAAAESERFVRQGTGN
DEAGAASADVVSLTCPVAAGECAGPADSGDALLE
RNYPTGAEFLGDGGDVSFSTRGTQNWTVERLLQA
HRQLEERGYVFVGYHGTFLEAAQSIVFGGVRARS
QDLDAIWRGFYIAGDPALAYGYAQDQEPDARGRI
RNGALLRVYVPRWSLPGFYRTGLTLAAPEAAGEV
ERLIGHPLPLRLDAITGPEEEGGRVTILGWPLAE
RTVVIPSAIPTDPRNVGGDLDPSSIPDKEQAISA
LPDYASQPGKPPREDLKHHHHHH

### SEQ ID NO:112

rEPA32 polynucleotide sequence—GlycoTag sequence SEQ ID NO: 145 in at residue 8274.

rEPA32 amino acid sequence-GlycoTag sequence
SEQ ID NO: 145 in at residue R274 (DsbA signal
sequence and GlycoTag underlined).
SEQ ID NO: 113
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTSAVTGYYLNHGTWP
KDNTSAGVASSPTDIKHRQPRGWEQLEQCGYPVQ
RLVALYLAARLSWNQVDQVIRNALASPGSGGDLG
EAIREQPEQARLALTLAAAESERFVRQGTGNDEA
GAASADVVSLTCPVAAGECAGPADSGDALLERNY
PTGAEFLGDGGDVSFSTRGTQNWTVERLLQAHRQ
LEERGYVFVGYHGTFLEAAQSIVFGGVRARSQDL
DAIWRGFYIAGDPALAYGYAQDQEPDARGRIRNG
ALLRVYVPRWSLPGFYRTGLTLAAPEAAGEVERL
IGHPLPLRLDAITGPEEEGGRVTILGWPLAERTV
VIPSAIPTDPRNVGGDLDPSSIPDKEQAISALPD
YASQPGKPPREDLK

### SEQ ID NO:114

rEPA33 polynucleotide sequence—GlycoTag sequence SEQ ID NO: 145 in at residue S408.

rEPA33 amino acid sequence-GlycoTag sequence
SEQ ID NO: 145 in at residue S408 (DsbA signal
sequence and GlycoTag underlined).
SEQ ID NO: 115
MKKIWLALAGLVLAFSASAAEEAFDLWNECAKAC
VLDLKDGVRSSRMSVDPAIADTNGQGVLHYSMVL
EGGNDALKLAIDNALSITSDGLTIRLEGGVEPNK
PVRYSYTRQARGSWSLNWLVPIGHEKPSNIKVFI
HELNAGNQLSHMSPIYTIEMGDELLAKLARDATF
FVRAHESNEMQPTLAISHAGVSVVMAQAQPRREK
RWSEWASGKVLCLLDPLDGVYNYLAQQRCNLDDT
WEGKIYRVLAGNPAKHDLDIKPTVISHRLHFPEG
GSLAALTAHQACHLPLEAFTRHRQPRGWEQLEQC
GYPVQRLVALYLAARLSWNQVDQVIRNALASPGS
GGDLGEAIREQPEQARLALTLAAAESERFVRQGT
GNDEAGAASADVVSLTCPVAAGECAGPADSGDAL
LERNYPTGAEFLGDGGDVSAVTGYYLNHGTWPKD
NTSAGVASSPTDIKFSTRGTQNWTVERLLQAHRQ
LEERGYVFVGYHGTFLEAAQSIVFGGVRARSQDL
DAIWRGFYIAGDPALAYGYAQDQEPDARGRIRNG
ALLRVYVPRWSLPGFYRTGLTLAAPEAAGEVERL
IGHPLPLRLDAITGPEEEGGRVTILGWPLAERTV
VIPSAIPTDPRNVGGDLDPSSIPDKEQAISALPD
YASQPGKPPREDLK

### SEQ ID NO:116

rEPA34 polynucleotide sequence—GlycoTag sequence SEQ ID NO: 145 in at residue A519.

rEPA34 amino acid sequence - GlycoTag sequence
SEQ ID NO: 145 in at residue A519 (DsbA signal
sequence and GlycoTag underlined).
SEQ ID NO: 117
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLEAFTRH
RQPRGWEQLEQCGYPVQRLVALYLAARLSWNQVDQVIRNALASPGSGGD
LGEAIREQPEQARLALTLAAAESERFVRQGTGNDEAGAASADVVSLTCP
VAAGECAGPADSGDALLERNYPTGAEFLGDGGDVSFSTRGTQNWTVERL
LQAHRQLEERGYVFVGYHGTFLEAAQSIVFGGVRARSQDLDAIWRGFYI
AGDPALAYGYAQDQEPDARGRIRNGALLRVYVPRWSLPGFYRTGLTLSA
VTGYYLNHGTWPKDNTSAGVASSPTDIKAPEAAGEVERLIGHPLPLRLD
AITGPEEEGGRVTILGWPLAERTVVIPSAIPTDPRNVGGDLDPSSIPDK
EQAISALPDYASQPGKPPREDLK

### SEQ ID NO:118

rEPA35 polynucleotide sequence—GlycoTag sequence SEQ ID NO: 146 in at residue S408.

rEPA35 amino acid sequence - GlycoTag sequence
SEQ ID NO: 146 in at residue S408 (DsbA signal
sequence and GlycoTag underlined).
SEQ ID NO: 119
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLEAFTRH
RQPRGWEQLEQCGYPVQRLVALYLAARLSWNQVDQVIRNALASPGSGGD
LGEAIREQPEQARLALTLAAAESERFVRQGTGNDEAGAASADVVSLTCP
VAAGECAGPADSGDALLERNYPTGAEFLGDGGDVGTWPKDNTSAGVASS
PTDIKFSTRGTQNWTVERLLQAHRQLEERGYVFVGYHGTFLEAAQSIVF
GGVRARSQDLDAIWRGFYIAGDPALAYGYAQDQEPDARGRIRNGALLRV
YVPRWSLPGFYRTGLTLAAPEAAGEVERLIGHPLPLRLDAITGPEEEGG
RVTILGWPLAERTVVIPSAIPTDPRNVGGDLDPSSIPDKEQAISALPDY
ASQPGKPPREDLK

### SEQ ID NO:120

rEPA36 polynucleotide sequence—GlycoTag sequence SEQ ID NO: 146 in at residue A519.

rEPA36 amino acid sequence - GlycoTag sequence
SEQ ID NO: 146 in at residue A519 (DsbA signal
sequence and GlycoTag underlined).
SEQ ID NO: 121
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLEAFTRH
RQPRGWEQLEQCGYPVQRLVALYLAARLSWNQVDQVIRNALASPGSGGD
LGEAIREQPEQARLALTLAAAESERFVRQGTGNDEAGAASADVVSLTCP
VAAGECAGPADSGDALLERNYPTGAEFLGDGGDVSFSTRGTQNWTVERL
LQAHRQLEERGYVFVGYHGTFLEAAQSIVFGGVRARSQDLDAIWRGFYI
AGDPALAYGYAQDQEPDARGRIRNGALLRVYVPRWSLPGFYRTGLTLGT
WPKDNTSAGVASSPTDIKAPEAAGEVERLIGHPLPLRLDAITGPEEEGG
RVTILGWPLAERTVVIPSAIPTDPRNVGGDLDPSSIPDKEQAISALPDY
ASQPGKPPREDLK

### SEQ ID NO:122

rEPA37 polynucleotide sequence—GlycoTag sequence SEQ ID NO: 146 in at residues 8274 and S408.

rEPA37 amino acid sequence - GlycoTag sequence
SEQ ID NO: 146 in at residues R274 and S408
(DsbA signal sequence and GlycoTags underlined).
SEQ ID NO: 123
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLEAFTGT
WPKDNTSAGVASSPTDIKHRQPRGWEQLEQCGYPVQRLVALYLAARLSW
NQVDQVIRNALASPGSGGDLGEAIREQPEQARLALTLAAAESERFVRQG
TGNDEAGAASADVVSLTCPVAAGECAGPADSGDALLERNYPTGAEFLGD
GGDVGTWPKDNTSAGVASSPTDIKFSTRGTQNWTVERLLQAHRQLEERG
YVFVGYHGTFLEAAQSIVFGGVRARSQDLDAIWRGFYIAGDPALAYGYA
QDQEPDARGRIRNGALLRVYVPRWSLPGFYRTGLTLAAPEAAGEVERLI
GHPLPLRLDAITGPEEEGGRVTILGWPLAERTVVIPSAIPTDPRNVGGD
LDPSSIPDKEQAISALPDYASQPGKPPREDLK

### SEQ ID NO:124

rEPA38 polynucleotide sequence—GlycoTag sequence SEQ ID NO: 146 in at residues 8274 and A519.

rEPA38 amino acid sequence - GlycoTag sequence
SEQ ID NO: 146 in at residues R274 and A519
(DsbA signal sequence and GlycoTags underlined).
SEQ ID NO: 125
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTGTWPKDNT
SAGVASSPTDIKHRQPRGWEQLEQCGYPVQRLVALYLAARLSWNQVDQVI
RNALASPGSGGDLGEAIREQPEQARLALTLAAAESERFVRQGTGNDEAGA
ASADVVSLTCPVAAGECAGPADSGDALLERNYPTGAEFLGDGGDVSFSTR
GTQNWTVERLLQAHRQLEERGYVFVGYHGTFLEAAQSIVFGGVRARSQDL
DAIWRGFYIAGDPALAYGYAQDQEPDARGRIRNGALLRVYVPRWSLPGFY
RTGLTLGTWPKDNTSAGVASSPTDIKAPEAAGEVERLIGHPLPLRLDAIT
GPEEEGGRVTILGWPLAERTVVIPSAIPTDPRNVGGDLDPSSIPDKEQAI
SALPDYASQPGKPPREDLK

### SEQ ID NO:126

rEPA39 polynucleotide sequence—GlycoTag sequence SEQ ID NO: 146 in at residues S408 and A519.

rEPA39 amino acid sequence - GlycoTag sequence
SEQ ID NO: 146 in at residues S408 and A519
(DsbA signal sequence and GlycoTags underlined).
SEQ ID NO: 127
MKKIWLALAGLVLAFSASAAEEAFDLWNECAKACVLDLKDGVRSSRMSV
DPAIADTNGQGVLHYSMVLEGGNDALKLAIDNALSITSDGLTIRLEGGV
EPNKPVRYSYTRQARGSWSLNWLVPIGHEKPSNIKVFIHELNAGNQLSH
MSPIYTIEMGDELLAKLARDATFFVRAHESNEMQPTLAISHAGVSVVMA
QAQPRREKRWSEWASGKVLCLLDPLDGVYNYLAQQRCNLDDTWEGKIYR
VLAGNPAKHDLDIKPTVISHRLHFPEGGSLAALTAHQACHLPLEAFTRH
RQPRGWEQLEQCGYPVQRLVALYLAARLSWNQVDQVIRNALASPGSGGD
LGEAIREQPEQARLALTLAAAESERFVRQGTGNDEAGAASADVVSLTCP
VAAGECAGPADSGDALLERNYPTGAEFLGDGGDVGTWPKDNTSAGVASS
PTDIKFSTRGTQNWTVERLLQAHRQLEERGYVFVGYHGTFLEAAQSIVF
GGVRARSQDLDAIWRGFYIAGDPALAYGYAQDQEPDARGRIRNGALLRV
YVPRWSLPGFYRTGLTLGTWPKDNTSAGVASSPTDIKAPEAAGEVERLI
GHPLPLRLDAITGPEEEGGRVTILGWPLAERTVVIPSAIPTDPRNVGGD
LDPSSIPDKEQAISALPDYASQPGKPPREDLK

### SEQ ID NO:128

rEPA40 polynucleotide sequence—GlycoTag sequence SEQ ID NO: 145 at N-terminus.

rEPA40 amino acid sequence - GlycoTag sequence
SEQ ID NO: 145 at N-terminus (DsbA signal
sequence underlined, GlycoTag double underlined).
SEQ ID NO: 129
MKKIWLALAGLVLAFSASASSAVTGYYLNHGTWPKDNSAGVASSPTDIK
AEEAFDLWNECAKACVLDLKDGVRSSRMSVDPAIADTNGQGVLHYSMVL
EGGNDALKLAIDNALSITSDGLTIRLEGGVEPNKPVRYSYTRQARGSWS
LNWLVPIGHEKPSNIKVFIHELNAGNQLSHMSPIYTIEMGDELLAKLAR
DATFFVRAHESNEMQPTLAISHAGVSVVMAQAQPRREKRWSEWASGKVL
CLLDPLDGVYNYLAQQRCNLDDTWEGKIYRVLAGNPAKHDLDIKPTVIS
HRLHFPEGGSLAALTAHQACHLPLEAFTRHRQPRGWEQLEQCGYPVQRL
VALYLAARLSWNQVDQVIRNALASPGSGGDLGEAIREQPEQARLALTLA
AAESERFVRQGTGNDEAGAASADVVSLTCPVAAGECAGPADSGDALLER
NYPTGAEFLGDGGDVSFSTRGTQNWTVERLLQAHRQLEERGYVFVGYHG
TFLEAAQSIVFGGVRARSQDLDAIWRGFYIAGDPALAYGYAQDQEPDAR
GRIRNGALLRVYVPRWSLPGFYRTGLTLAAPEAAGEVERLIGHPLPLRL
DAITGPEEEGGRVTILGWPLAERTVVIPSAIPTDPRNVGGDLDPSSIPD
KEQAISALPDYASQPGKPPREDLK

### SEQ ID NO:130

rEPA41 polynucleotide sequence—GlycoTag sequence SEQ ID NO: 146 in at residue 8274.

rEPA41 amino acid sequence - GlycoTag sequence
SEQ ID NO: 146 in at residue R274 (DsbA signal
sequence and GlycoTag underlined).
SEQ ID NO: 131
MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTGTWPKDNT
SAGVASSPTDIKHRQPRGWEQLEQCGYPVQRLVALYLAARLSWNQVDQVI
RNALASPGSGGDLGEAIREQPEQARLALTLAAAESERFVRQGTGNDEAGA
ASADVVSLTCPVAAGECAGPADSGDALLERNYPTGAEFLGDGGDVSFSTR
GTQNWTVERLLQAHRQLEERGYVFVGYHGTFLEAAQSIVFGGVRARSQDL
DAIWRGFYIAGDPALAYGYAQDQEPDARGRIRNGALLRVYVPRWSLPGFY
RTGLTLAAPEAAGEVERLIGHPLPLRLDAITGPEEEGGRVTILGWPLAER
TVVIPSAIPTDPRNVGGDLDPSSIPDKEQAISALPDYASQPGKPPREDLK

### SEQ ID NO:132

rEPA42 polynucleotide sequence—GlycoTag sequence SEQ ID NO: 145 in at residues 8274 and A519.

rEPA42 amino acid sequence - GlycoTag sequence
SEQ ID NO: 145 in at residues R274 and A519
(DsbA signal sequence and GlycoTags underlined).
SEQ ID NO: 133
MKKIWLALAGLVLAFSASAAEEAFDLWNECAKACVLDL
KDGVRSSRMSVDPAIADTNGQGVLHYSMVL
EGGNDALKLAIDNALSITSDGLTIRLEGGVEP
NKPVRYSYTRQARGSWSLNWLVPIGHEKPSNIKVFI
HELNAGNQLSHMSPIYTIEMGDELLAKLARDAT
FFVRAHESNEMQPTLAISHAGVSVVMAQAQPRREK
RWSEWASGKVLCLLDPLDGVYNYLAQQRCNLDDTWE
GKIYRVLAGNPAKHDLDIKPTVISHRLHFPEG
GSLAALTAHQACHLPLEAFTSAVTGYYLNHGTWPKDNT
SAGVASSPTDIKHRQPRGWEQLEQCGYPVQ
RLVALYLAARLSWNQVDQVIRNALASPGSGGDLGEAI
REQPEQARLALTLAAAESERFVRQGTGNDEA
GAASADVVSLTCPVAAGECAGPADSGDALLERNY
PTGAEFLGDGGDVSFSTRGTQNWTVERLLQAHRQ
LEERGYVFVGYHGTFLEAAQSIVFGGVRARSQDL
DAIWRGFYIAGDPALAYGYAQDQEPDARGRIRNG
ALLRVYVPRWSLPGFYRTGLSAVTGYYLNHGTW
PKDNTSAGVASSPTDIKAPEAAGEVERLIGHPL
PLRLDAITGPEEEGGRVTILGWPLAERTVVIPSAI
PTDPRNVGGDLDPSSIPDKEQAISALPDYASQP
GKPPREDLK

### SEQ ID NO:134

rEPA43 polynucleotide sequence—GlycoTag sequence SEQ ID NO: 140 at N-terminus and C-terminus.

rEPA43 amino acid sequence - GlycoTag sequence
SEQ ID NO: 140 at N-terminus and C-terminus
(DsbA signal sequence and
6xHis Tag (SEQ ID NO: 217)
underlined; GlycoTags double underlined).
SEQ ID NO: 135
MKKIWLALAGLVLAFSASASAVTEYYLNHGEWPGNNT
SAGVATSSEIKAEEAFDLWNECAKACVLDLK
DGVRSSRMSVDPAIADTNGQGVLHYSMVLEGGNDALK
LAIDNALSITSDGLTIRLEGGVEPNKPVRYS
YTRQARGSWSLNWLVPIGHEKPSNIKVFIHEL
NAGNQLSHMSPIYTIEMGDELLAKLARDATFFVRAH
ESNEMQPTLAISHAGVSVVMAQAQPRREKRWSEWASG
KVLCLLDPLDGVYNYLAQQRCNLDDTWEGKI
YRVLAGNPAKHDLDIKPTVISHRLHFPEGGSLAALTA
HQACHLPLEAFTRHRQPRGWEQLEQCGYPVQ
RLVALYLAARLSWNQVDQVIRNALASPGSGGDLGE
AIREQPEQARLALTLAAAESERFVRQGTGNDEA
GAASADVVSLTCPVAAGECAGPADSGDALLERNYPT
GAEFLGDGGDVSFSTRGTQNWTVERLLQAHRQ
LEERGYVFVGYHGTFLEAAQSIVFGGVRARSQDLD
AIWRGFYIAGDPALAYGYAQDQEPDARGRIRNG
ALLRVYVPRWSLPGFYRTGLTLAAPEAAGEVERLIGH
PLPLRLDAITGPEEEGGRVTILGWPLAERTV
VIPSAIPTDPRNVGGDLDPSSIPDKEQAISALPDY
ASQPGKPPREDLKSAVTEYYLNHGEWPGNNTSA
GVATSSEIKHHHHHH
Mature rEPA43 amino acid sequence (i.e., signal
sequence removed) - GlycoTag sequence SEQ ID
NO: 140 at N-terminus and C-terminus (6xHis
Tag (SEQ ID NO: 217) underlined;
GlycoTags double underlined).
SEQ ID NO: 136
SAVTEYYLNHGEWPGNNTSAGVATSSEIKAEEAFDLW
NECAKACVLDLKDGVRSSRMSVDPAIADTNG
QGVLHYSMVLEGGNDALKLAIDNALSITSDGLTI
RLEGGVEPNKPVRYSYTRQARGSWSLNWLVPIGH
EKPSNIKVFIHELNAGNQLSHMSPIYTIEMGDELLAK
LARDATFFVRAHESNEMQPTLAISHAGVSVV
MAQAQPRREKRWSEWASGKVLCLLDPLDGVYNYLAQQRCNL
DDTWEGKIYRVLAGNPAKHDLDIKPTV
ISHRLHFPEGGSLAALTAHQACHLPLEAFTRHR
QPRGWEQLEQCGYPVQRLVALYLAARLSWNQVDQV
IRNALASPGSGGDLGEAIREQPEQARLALTLAAAE
SERFVRQGTGNDEAGAASADVVSLTCPVAAGEC
AGPADSGDALLERNYPTGAEFLGDGGDVSFST
RGTQNWTVERLLQAHRQLEERGYVFVGYHGTFLEAA
QSIVFGGVRARSQDLDAIWRGFYIAGDPALAYGYA
QDQEPDARGRIRNGALLRVYVPRWSLPGFYRTG
LTLAAPEAAGEVERLIGHPLPLRLDAITGPEEEGGR
VTILGWPLAERTVVIPSAIPTDPRNVGGDLDP
SSIPDKEQAISALPDYASQPGKPPREDLKSAV
TEYYLNHGEWPGNNTSAGVATSSEIKHHHHHH

### SEQ ID NO: 137

Neisseria meningitidis MC58 PilE amino acid sequence (mature sequence; signal sequence removed). Corresponds to NCBI Accession NP_273084.1.

Neisseria meningitidis MC58 PilE amino acid
sequence (signal sequence underlined).
Corresponds to NCBI Accession NP_273084.1.
SEQ ID NO: 138
MNTLQKGFTLIELMIVIAIVGILAAVALPAYQDYTARA
QVSEAILLAEGQKSAVTEYYLNHGEWPGNN
TSAGVATSSEIKGKYVKSVEVKNGVVTAQMASSNVN
NEIKGKKLSLWAKRQNGSVKWFCGQPVTRDKA
KAANDDVTAAAAANGKKIDTKHLPSTCRDASDAS
Neisseria meningitidis MC58 PilE polynucleotide
sequence. Corresponds to 17741-17229 of NCBI
Reference Sequence NC_003112.2.
SEQ ID NO: 139
ATGAACACCCTTCAAAAAGGTTTTACCCTTATCGA
GCTGATGATTGTGATTGCCATCGTCGGCATTTT
GGCGGCAGTCGCCCTTCCTGCTTATCAAGACTACACAG
CCCGCGCACAAGTTTCCGAAGCCATTCTTT
TGGCCGAAGGTCAAAAATCAGCCGTTACCGAGTATTACCT
GAATCACGGCGAATGGCCCGGCAACAAC
ACTTCTGCCGGCGTGGCAACCTCCTCTGAAATCA
AAGGCAAATATGTTAAAAGCGTTGAAGTCAAAAA
CGGCGTCGTTACCGCCCAAATGGCTTCAAGCAAC
GTAAACAACGAAATCAAAGGCAAAAAACTCTCCC
TGTGGGCCAAGCGTCAAAACGGTTCGGTAAAATGG
TTCTGCGGACAGCCGGTTACGCGCGACAAAGCC
AAAGCCGCCAACGACGACGTTACCGCCGCCGCCGCC
GCCAACGGTAAGAAGATTGACACCAAGCACCT
GCCGTCAACCTGCCGCGACGCAAGTGATGCCAGCTAA

### SEQ ID NO: 140

Neisseria meningitidis PilE GlycoTag amino acid sequence (corresponding to residues 45-73 of SEQ ID NO: 137; 29 amino acid long). E.g. Ser Ala Val Thr Glu Tyr Tyr Leu Asn His Gly Glu Trp Pro Gly Asn Asn Thr Ser Ala Gly Val Ala Thr Ser Ser Glu Ile Lys

### SEQ ID NO: 141

Neisseria meningitidis PilE GlycoTag amino acid sequence (corresponding to residues 55-73 of SEQ ID NO: 137; 19 amino acid long). E.g. Gly Glu Trp Pro Gly Asn Asn Thr Ser Ala Gly Val Ala Thr Ser Ser Glu Ile Lys

### SEQ ID NO: 142

Neisseria meningitidis PilE GlycoTag amino acid sequence (corresponding to residues 55-66 of SEQ ID NO: 137; 12 amino acid long). E.g. Gly Glu Trp Pro Gly Asn Asn Thr Ser Ala Gly Val

### SEQ ID NO: 143

Neisseria gonorrhoeae Pilin (NgPilin) amino acid sequence. Corresponds to NCBI GenBank CNT62005.1.

### SEQ ID NO: 144

Neisseria gonorrhoeae Pilin (NgPilin) polynucleotide sequence. Corresponds to NCBI GenBank CNT62005.1.

### SEQ ID NO: 145

Neisseria gonorrhoeae GlycoTag amino acid sequence (corresponding to residues 52-81 of SEQ ID NO: 143; 30 amino acid long). E. g. Ser Ala Val Thr Gly Tyr Tyr Leu Asn His Gly Thr Trp Pro Lys Asp Asn Thr Ser Ala Gly Val Ala Ser Ser Pro Thr Asp Ile Lys

### SEQ ID NO: 146

Neisseria gonorrhoeae GlycoTag amino acid sequence (corresponding to residues 62-81 of SEQ ID NO: 143; 20 amino acid long). E. g. Gly Thr Trp Pro Lys Asp Asn Thr Ser Ala Gly Val Ala Ser Ser Pro Thr Asp Ile Lys

### SEQ ID NO: 147

Neisseria gonorrhoeae GlycoTag amino acid sequence (corresponding to residues 62-73 of SEQ ID NO: 143; 12 amino acid long). E.g. Gly Thr Trp Pro Lys Asp Asn Thr Ser Ala Gly Val

### SEQ ID NO: 148

Neisseria lactamica 020-06 Pilin (NlPilin) amino acid sequence. Corresponds to NCBI GenBank CBN86420.1.

### SEQ ID NO: 149

Neisseria lactamica 020-06 Pilin (NlPilin) polynucleotide sequence. Corresponds to NCBI GenBank CBN86420.1.

### SEQ ID NO: 150

Neisseria lactamica 020-06 GlycoTag amino acid sequence (corresponding to residues 52-86 of SEQ ID NO: 148; 35 amino acid long). E.g. Ala Ala Val Val Glu Tyr Tyr Ser Asp Asn Gly Thr Phe Pro Ala Gln Asn Ala Ser Ala Gly Ile Ala Thr Ala Ser Ala Ile Thr Gly Lys Tyr Val Ala Lys

### SEQ ID NO: 151

Neisseria lactamica 020-06 GlycoTag amino acid sequence (corresponding to residues 62-73 of SEQ ID NO: 148; 12 amino acid long). E.g. Gly Thr Phe Pro Ala Gln Asn Ala Ser Ala Gly Ile

### SEQ ID NO: 152

Neisseria elongata subsp. glycolytica ATCC 29315 Pilin (NePilin) polynucleotide sequence. Corresponds to NCBI GenBank EFE49588.1.

### SEQ ID NO:153

Neisseria elongata subsp. glycolytica ATCC 29315 Pilin (NePilin) amino acid sequence. Corresponds to NCBI GenBank EFE49588.1. 100% identity to SEQ ID NO: 186.

### SEQ ID NO: 154

Neisseria elongata subsp. glycolytica ATCC 29315 GlycoTag amino acid sequence (corresponding to residues 52-97 of SEQ ID NO: 153; 45 amino acid long).

### SEQ ID NO: 155

Neisseria bacilliformis ATCC BAA-1200 (NbPilin) polynucleotide sequence. Corresponds to NCBI GenBank EGF11985.1.

### SEQ ID NO:156

Neisseria bacilliformis ATCC BAA-1200 (NbPilin) amino acid sequence. Corresponds to NCBI GenBank EGF11985.1.

### SEQ ID NO: 157

Neisseria bacilliformis ATCC BAA-1200 GlycoTag amino acid sequence (corresponding to residues 57-93 of SEQ ID NO: 156; 37 amino acid long).

### SEQ ID NO: 158

Neisseria mucosa ATCC 25996 (NmuPilin) polynucleotide sequence. Corresponds to NCBI GenBank EFC89512.1.

### SEQ ID NO:159

Neisseria mucosa ATCC 25996 (NmuPilin) amino acid sequence. Corresponds to NCBI GenBank EFC89512.1.

### SEQ ID NO: 160

Neisseria mucosa ATCC 25996 GlycoTag amino acid sequence (corresponding to residues 52-92 of SEQ ID NO: 159; 41 amino acids long).

### SEQ ID NO: 161

Neisseria shayeganii 871 (NsPilin) polynucleotide sequence. Corresponds to NCBI GenBank EGY51595.1.

### SEQ ID NO:162

Neisseria shayeganii 871 (NsPilin) amino acid sequence. Corresponds to NCBI GenBank EGY51595.1. 100% identity to SEQ ID NOs: 177 and 179.

### SEQ ID NO: 163

Neisseria shayeganii 871 GlycoTag amino acid sequence (corresponding to residues 53-83 of SEQ ID NO: 162; 31 amino acids long). E.g. Gly Ala Val Thr Glu Tyr Glu Ala Asp Lys Gly Val Phe Pro Thr Ser Asn Ala Ser Ala Gly Val Ala Ala Ala Ala Asp Ile Asn Gly Lys

### SEQ ID NO: 164

Neisseria shayeganii 871 GlycoTag amino acid sequence (corresponding to residues 63-74 of SEQ ID NO: 162; 12 amino acids long). E.g. Gly Val Phe Pro Thr Ser Asn Ala Ser Ala Gly Val

### SEQ ID NO: 165

Neisseria lactamica ATCC 23970 Pilin amino acid sequence. Corresponds to NCBI GenBank EEZ75637.1.

### SEQ ID NO: 166

Neisseria gonorrhoeae F62 Pilin amino acid sequence. Corresponds to NCBI GenBank EFF40919.1.

### SEQ ID NO: 167

Neisseria cinereal ATCC 14685 Pilin amino acid sequence. Corresponds to NCBI GenBank EEZ70774.1.

### SEQ ID NO: 168

Neisseria cinereal ATCC 14685 Pilin amino acid sequence. Corresponds to NCBI GenBank EEZ70775.1.

### SEQ ID NO: 169

Neisseria mucosa Pilin amino acid sequence. Corresponds to NCBI GenBank KGJ31398.1.

### SEQ ID NO: 170

Neisseria mucosa Pilin amino acid sequence. Corresponds to NCBI GenBank KGJ31397.1.

### SEQ ID NO: 171

Neisseria flavescens NRL30031/H210 Pilin amino acid sequence. Corresponds to NCBI GenBank EEG33288.1.

### SEQ ID NO: 172

Neisseria mucosa ATCC 25996 Pilin amino acid sequence. Corresponds to NCBI GenBank EFC89512.1.

### SEQ ID NO: 173

Neisseria mucosa ATCC 25996 Pilin amino acid sequence. Corresponds to NCBI GenBank EFC89511.1.

### SEQ ID NO: 174

Neisseria sp oral taxon 014 str. F0314 Pilin amino acid sequence. Corresponds to NCBI GenBank EFI23295.1.

### SEQ ID NO: 175

Neisseria sp oral taxon 014 str. F0314 Pilin amino acid sequence. Corresponds to NCBI GenBank EFI23294.1.

### SEQ ID NO: 176

Neisseria arctica Pilin amino acid sequence. Corresponds to NCBI GenBank KLT73057.1.

### SEQ ID NO: 177

Neisseria shayeganii 871 Pilin amino acid sequence. Corresponds to NCBI GenBank EGY51595.1. 100% identity to SEQ ID NOs: 162 and 179.

### SEQ ID NO: 178

Neisseria shayeganii 871 Pilin amino acid sequence. Corresponds to NCBI GenBank. EGY51594 (=ID 180)

### SEQ ID NO: 179

Neisseria shayeganii 871 Pilin amino acid sequence. Corresponds to NCBI GenBank EGY51595.1. 100% identity to SEQ ID NOs: 162 and 177.

### SEQ ID NO: 180

Neisseria shayeganii 871 Pilin amino acid sequence. Corresponds to NCBI GenBank EGY51594.1.

### SEQ ID NO: 181

Neisseria sp. 83E34 Pilin amino acid sequence. Corresponds to NCBI GenBank KPN71218.1.

### SEQ ID NO: 182

Neisseria sp. 83E34 Pilin amino acid sequence. Corresponds to NCBI GenBank KPN71186.1.

### SEQ ID NO: 183

Neisseria wadsworthii 9715 Pilin amino acid sequence. Corresponds to NCBI GenBank EGZ51246.1.

### SEQ ID NO:184

Neisseria wadsworthii 9715 Pilin amino acid sequence. Corresponds to NCBI GenBank EGZ51247.1.

### SEQ ID NO: 185

Neisseria elongata subsp. glycolytica ATCC 29315 Pilin amino acid sequence. Corresponds to NCBI GenBank EFE49587.1.

### SEQ ID NO: 186

Neisseria elongata subsp. glycolytica ATCC 29315 Pilin amino acid sequence. Corresponds to NCBI GenBank EFE49588.1. 100% identity to SEQ ID NO: 153.

### SEQ ID NO: 187

Neisseria bacilliformis ATCC BAA-1200 Pilin amino acid sequence. Corresponds to NCBI GenBank EGF04823.1.

### SEQ ID NO: 188

Neisseria bacilliformis ATCC BAA-1200 Pilin amino acid sequence. Corresponds to NCBI GenBank EGF11985.1.

### SEQ ID NO: 189

Neisseria bacilliformis ATCC BAA-1200 Pilin amino acid sequence. Corresponds to NCBI GenBank EGF12096.1.

### SEQ ID NO: 190

Neisseria sp. oral taxon 020 str. F0370 Pilin amino acid sequence. Corresponds to NCBI GenBank EKY04118.1.

### SEQ ID NO: 191

Neisseria sp. oral taxon 020 str. F0370 Pilin amino acid sequence. Corresponds to NCBI GenBank EKY04120.1.

### SEQ ID NO: 192

Neisseria sp. 74A18 Pilin amino acid sequence. Corresponds to NCBI GenBank KPN73545.1.

### SEQ ID NO: 193

Neisseria sp. 74A18 Pilin amino acid sequence. Corresponds to NCBI GenBank KPN73546.1.

### SEQ ID NO: 194

Neisseria weaver ATCC 51223 Pilin amino acid sequence. Corresponds to NCBI GenBank EGV37979.1.

### SEQ ID NO: 195

Neisseria macacae ATCC 33926 Pilin amino acid sequence. Corresponds to NCBI GenBank EGQ74605.1.

### SEQ ID NO: 196

Neisseria macacae ATCC 33926 Pilin amino acid sequence. Corresponds to NCBI GenBank EGQ74606.1.

### SEQ ID NO: 197

AcrA polynucleotide sequence (including pelB signal sequence).

AcrA amino acid sequence
(pelB signal sequence underlined).
SEQ ID NO: 198
MKYLLPTAAAGLLLLAAQPAMAMHMSKEEAPKIQMP
PQPVTTMSAKSEDLPLSFTYPAKLVSDYDVII
KPQVSGVIVNKLFKAGDKVKKGQTLFIIEQDKFKA
SVDSAYGQALMAKATFENASKDFNRSKALFSKS
AISQKEYDSSLATFNNSKASLASARAQLANARIDLDHTE
IKAPFDGTIGDALVNIGDYVSASTTELVR
VTNLNPIYADFFISDTDKLNLVRNTQSGKWDLDS
IHANLNLNGETVQGKLYFIDSVIDANSGTVKAKA
VFDNNNSTLLPGAFATITSEGFIQKNGFKVPQIGV
KQDQNDVYVLLVKNGKVEKSSVHISYQNNEYAI
IDKGLQNGDKIILDNFKKIQVGSEVKEIGAQLE
mAcrA amino acid sequence - GlycoTag sequence
SEQ ID NO: 140 at C-terminus (pelB signal
sequence and GlycoTag SEQ ID NO: 140 underlined;
6,(1-lis-tag (SEQ ID NO: 217) double underlined).
SEQ ID NO: 199
MKYLLPTAAAGLLLLAAQPAMAMHMSKEEAPKIQMP
PQPVTTMSAKSEDLPLSFTYPAKLVSDYDVII
KPQVSGVIVNKLFKAGDKVKKGQTLFIIEQDKFK
ASVDSAYGQALMAKATFENASKDFLRSKALFSKS
AISQKEYDSSLATFNNSKASLASARAQLANARIDLDHTE
IKAPFDGTIGDALVNIGDYVSASTTELVR
VTNLNPIYADFFISDTDKLNLVRNTQSGKWDL
DSIHANLNLNGETVQGKLYFIDSVIDANSGTVKAKA
VFDNNNSTLLPGAFATITSEGFIQKNGFKVPQIGVK
QDQNDVYVLLVKNGKVEKSSVHISYQNNEYAI
IDKGLQNGDKIILDNFKKIQVGSEVKEIGAQLE
SAVTEYYLNHGEWPGNNTSAGVATSSEIKHHHHHH

### SEQ ID NO: 200

PcrV polynucleotide sequence (including LtIIb signal sequence).

PcrV amino acid sequence
(LtIIb signal sequence underlined).
SEQ ID NO: 201
MSFKKIIKAFVIMAALVSVQAHAAEVRNLNAARELFLDELLAASAAPAS
AEQEELLALLRSERIVLAHAGQPLSEAQVLKALAWLLAANPSAPPGQGL
EVLREVLQARRQPGAQWDLREFLVSAYFSLHGRLDEDVIGVYKDVLQTQ
DGKRKALLDELKALTAELKVYSVIQSQINAALSAKQGIRIDAGGIDLVD
PTLYGYAVGDPRWKDSPEYALLSNLDTFSGKLSIKDFLSGSPKQSGELK
GLSDEYPFEKDNNPVGNFATTVSDRSRPLNDKVNEKTTLLNDTSSRYNS
AVEALNRFIQKYDSVLRDILSAIGS
mPcrV amino acid sequence - GlycoTag sequence
SEQ ID NO: 140 at C-terminus (LtIIb signal
sequence and GlycoTag underlined; 6,(1-lis-
tag (SEQ ID NO: 217) double underlined).
SEQ ID NO: 202
MSFKKIIKAFVIMAALVSVQAHAAEVRNLNAARELFLDELLAASAAPAS
AEQEELLALLRSERIVLAHAGQPLSEAQVLKALAWLLAANPSAPPGQGL
EVLREVLQARRQPGAQWDLREFLVSAYFSLHGRLDEDVIGVYKDVLQTQ
DGKRKALLDELKALTAELKVYSVIQSQINAALSAKQGIRIDAGGIDLVD
PTLYGYAVGDPRWKDSPEYALLSNLDTFSGKLSIKDFLSGSPKQSGELK
GLSDEYPFEKDNNPVGNFATTVSDRSRPLNDKVNEKTTLLNDTSSRYNS
AVEALNRFIQKYDSVLRDILSAIGSSAVTEYYLNHGEWPGNNTSAGVAT
SSEIKHHHHHH

### SEQ ID NO: 203

Crm197 polynucleotide sequence (including DsbA signal sequence and GlycoTag sequence SEQ ID NO: 140 at C-terminus).

mCrm197 amino acid sequence - GlycoTag
sequence SEQ ID NO: 140 at C-terminus (DsbA signal
sequence and GlycoTag underlined; 6xHis-
tag (SEQ ID NO: 217) double underlined).
SEQ ID NO: 204
MKKIWLALAGLVLAFSASAGADDVVDSSKSFVMENFSSYHGT
KPGYVDSIQKGIQKPKSGTQGNYDDD
WKEFYSTDNKYDAAGYSVDNENPLSGKAGGVVKVTYPGLTKV
LALKVDNAETIKKELGLSLTEPLMEQ
VGTEEFIKRFGDGASRVVLSLPFAEGSSSVEYINNWEQA
KALSVELEINFETRGKRGQDAMYEYMAQA
CAGNRVRRSVGSSLSCINLDWDVIRDKTKTKIESLKEH
GPIKNKMSESPNKTVSEEKAKQYLEEFHQT
ALEHPELSELKTVTGTNPVFAGANYAAWAVNVAQVIDSET
ADNLEKTTAALSILPGIGSVMGIADGAV
HHNTEEIVAQSIALSSLMVAQAIPLVGELVDIGFAAYNFVES
IINLFQVVHNSYNRPAYSPGHKTQPF
LHDGYAVSWNTVEDSIIRTGFQGESGHDIKITAENTPLPIAGVL
LPTIPGKLDVNKSKTHISVNGRKI
RMRCRAIDGDVTFCRPKSPVYVGNGVHANLHVAFHRSSSEK
IHSNEISSDSIGVLGYQKTVDHTKVNS
KLSLFFEIKSGSSAVTEYYLNHGEWPGNNTSAGVAT
SSEIKHHHHHH
Crm197 amino acid sequence
(DsbA signal sequence underlined).
SEQ ID NO: 205
MKKIWLALAGLVLAFSASAGADDVVDSSKSFVMENFSSYHGTKPGYVDS
IQKGIQKPKSGTQGNYDDD
WKEFYSTDNKYDAAGYSVDNENPLSGKAGGVVKVTYPGLTKVLALKVDNA
ETIKKELGLSLTEPLMEQ
VGTEEFIKRFGDGASRVVLSLPFAEGSSSVEYINNWEQAKALSVELEINF
ETRGKRGQDAMYEYMAQA
CAGNRVRRSVGSSLSCINLDWDVIRDKTKTKIESLKEHGPIKNKMSESPN
KTVSEEKAKQYLEEFHQT
ALEHPELSELKTVTGTNPVFAGANYAAWAVNVAQVIDSETADNLEKTTAA
LSILPGIGSVMGIADGAV
HHNTEEIVAQSIALSSLMVAQAIPLVGELVDIGFAAYNFVESIINL
FQVVHNSYNRPAYSPGHKTQPF
LHDGYAVSWNTVEDSIIRTGFQGESGHDIKITAENTPLPIAGVLLPTIPG
KLDVNKSKTHISVNGRKI
RMRCRAIDGDVTFCRPKSPVYVGNGVHANLHVAFHRSSSEKIHSNEIS
SDSIGVLGYQKTVDHTKVNS
KLSLFFEIKSGS

### SEQ ID NO: 206

m2Crm197 polynucleotide sequence (including DsbA signal sequence and GlycoTag sequence SEQ ID NO: 140 at N-terminus and C-terminus)

M2Crm197 amino acid sequence - GlycoTag sequence
SEQ ID NO: 140 at N-terminus and C-terminus
(DsbA signal sequence and 6,(Elis-tag (SEQ ID
NO: 217) underlined; GlycoTags double underlined).
SEQ ID NO: 207
MKKIWLALAGLVLAFSASASAVTEYYLNHGEWPGNNT
SAGVATSSEIKGADDVVDSSKSFVMENFSSY
HGTKPGYVDSIQKGIQKPKSGTQGNYDDDWKEFYST
DNKYDAAGYSVDNENPLSGKAGGVVKVTYPGL
TKVLALKVDNAETIKKELGLSLTEPLMEQVGTEE
FIKRFGDGASRVVLSLPFAEGSSSVEYINNWEQA
KALSVELEINFETRGKRGQDAMYEYMAQACAGNRVRR
SVGSSLSCINLDWDVIRDKTKTKIESLKEHG
PIKNKMSESPNKTVSEEKAKQYLEEFHQTALE
HPELSELKTVTGTNPVFAGANYAAWAVNVAQVIDSE
TADNLEKTTAALSILPGIGSVMGIADGAVHH
NTEEIVAQSIALSSLMVAQAIPLVGELVDIGFAAYNF
VESIINLFQVVHNSYNRPAYSPGHKTQPFLHDGYAVS
WNTVEDSIIRTGFQGESGHDIKITAENTPLP
IAGVLLPTIPGKLDVNKSKTHISVNGRKIRMRC
RAIDGDVTFCRPKSPVYVGNGVHANLHVAFHRSSS
EKIHSNEISSDSIGVLGYQKTVDHTKVNSKLSLFFE
IKSGSSAVTEYYLNHGEWPGNNTSAGVATSSE
IKHHHHHH

### SEQ ID NO: 208

Plesiomonas shigelloides O17 (i.e., Shigella sonnei) O-antigen WbgT amino acid sequence corresponding to NCBI GenBank Accession No. AAG17408.1. [102]

### SEQ ID NO: 209

Plesiomonas shigelloides O17 (i.e., Shigella sonnei) O-antigen WbgU amino acid sequence corresponding to NCBI GenBank Accession No. AAG17409.1. [102]

### SEQ ID NO: 210

Plesiomonas shigelloides O17 (i.e., Shigella sonnei) O-antigen Wzx amino acid sequence corresponding to NCBI GenBank Accession No. AAG17410.1. [102]

### SEQ ID NO: 211

Plesiomonas shigelloides O17 (i.e., Shigella sonnei) O-antigen Wzy amino acid sequence corresponding to NCBI GenBank Accession No. AAG17411.1. [102]

### SEQ ID NO: 212

Plesiomonas shigelloides O17 (i.e., Shigella sonnei) O-antigen WbgV amino acid sequence corresponding to NCBI GenBank Accession No. AAG17412.1. [102]

### SEQ ID NO: 213

Plesiomonas shigelloides O17 (i.e., Shigella sonnei) O-antigen WbgW amino acid sequence corresponding to NCBI GenBank Accession No. AAG17413.1. [102]

### SEQ ID NO: 214

Plesiomonas shigelloides O17 (i.e., Shigella sonnei) O-antigen WbgX amino acid sequence corresponding to NCBI GenBank Accession No. AAG17414.1. [102]

### SEQ ID NO: 215

Plesiomonas shigelloides O17 (i.e., Shigella sonnei) O-antigen WbgY amino acid sequence corresponding to NCBI GenBank Accession No. AAG17415.1. [102]

### SEQ ID NO: 216

Plesiomonas shigelloides O17 (i.e., Shigella sonnei) O-antigen WbgZ amino acid sequence corresponding to NCBI GenBank Accession No. AAG17416.1. [102]

### SEQ ID NO: 217

### 6×His-tag

### TABLE OF REFERENCES

[1] Faridmoayer et al., “Functional Characterization of Bacterial Oligosaccharyltransferases Involved in O-Linked Protein Glycosylation,” J. Bacteriology, pp. 8088-8098, 2007.
[2] Tan et al., “Sugar coating: bacterial protein glycosylation and host-microbe interactions,” Trends in Bioch. Sci., vol. 40, no. 7, pp. 342-350, 2015.
[3] Faridmoayer et al., “Extreme Substrate Promiscuity of the Neisseria Oligosaccharyl Transferase Involved in Protein O-Glycosylation,” J. Biol. Chem., vol. 283, no. 50, pp. 34596-34604, 2008.
[4] Pan et al., “Biosynthesis of Conjugate Vaccines Using an O-Linked Glycosylation System,” mBio, vol. 7, no. 2, pp. e00443-16, 2016.
[5] Ihssen et al., “Production of glycoprotein vaccines in Escherichia coli,” Microbial Cell Factories, vol. 9, p. 61, 13 total pages, 2010.
[6] Institute of Biotechnology, Academy of Military Medical Sciences, China WO/2016/082597 (Jun. 2, 2016; English language equivalent EP3225690).
[7] Vik et al., “Broad spectrum O-linked protein glycosylation in the human pathogen Neisseria gonorrhoeae,” PNAS, vol. 106, no. 11, pp. 4447-4452, 2009.
[8] Ravenscroft et al., “Purification and characterization of a Shigella Conjugate Vaccine, Produced by Glycoengineering Escherichia coli,” Glycobiology, vol. 26, no. 1, pp. 51-62, 2016.
[9] Glycovaxyn AG, U.S. Pat. No. 8,846,342 (Sep. 30, 2014).
[10] Glycovaxyn AG, U.S. Pat. No. 8,895,014 (Nov. 25, 2014).
[11] ETH Zurich, U.S. Pat. No. 8,753,864 (Jun. 17, 2014).
[12] Glycovaxyn AG, U.S. Pat. No. 9,585,950 (Mar. 7, 2017).
[13] Wacker et al., “Substrate specificity of bacterial oligosaccharyltransferase suggests a common transfer mechanism for the bacterial and eukaryotic systems,” PNAS, vol. 103, pp. 7088-7093, 2006.
[14] Bentley, “Genetic analysis of the capsular biosynthetic locus from all 90 pneumococcal serotypes,” PLoS Gen, vol. 2, p. e31, 2006.
[15] Borud et al., “Genetic, Structural, and Antigenic Analysis of Glycan Diversity in the O-Linked Protein Glycosylation Systems of Human Neisseria Species,” J. Bacteriology, vol. 192, no. 11, pp. 2816-2829, 2010.
[16] Li et al., “Understanding protein glycosylation pathways in bacteria,” Future Microbiol., vol. 12, no. 1, pp. 59-72, 2017.
[17] Schulz et al., “Identification of Bacterial Protein O-Oligosaccharyltransferases and their glycoprotein substrates,” PLoS One, vol. 8, no. 5, p. e62768, 2013.
[18] Ruan et al., “The Waal, O-antigen lipopolysaccharide ligase has features in common with metal ion-independent inverting glycosyltransferases,” Glycobiology, vol. 22, no. 2, pp. 288-299, 2012.
[19] Aas et al., “Neisseria gonorrhoeae O-linked pilin glycosylation: functional analyses define both the biosynthetic pathway and glycan structure,” Molecular Microbiology, vol. 65, no. 3, pp. 607-624, 2007.
[20] Samuel & Reeves, “Biosynthesis of O-antigens: genes and pathways involved in nucleotide sugar precursor synthesis and O-antigen assembly,” Carbohydrate Research, vol. 338, pp. 2503-2519, 2003.
[21] May, “Percent Sequence Identity: The Need to Be Explicit,” Structure, vol. 12, pp. 737-738, 2004.
[22] Karlin et al., “Methods for assessing the statistical significance of molecular sequence features by using general scoring schemes,” PNAS, vol. 87, pp. 2264-2268, 1990.
[23] Karlin & Altschul, “Applications and statistics for multiple high-scoring segments in molecular sequences,” PNAS, vol. 90, pp. 5873-5877, 1993.
[24] Altschul et al., “Gapped BLAST and PSI-BLAST: a new generation of protein database search programs,” Nucleic Acids Research, vol. 25, no. 17, pp. 3389-3402, 1997.
[25] Altschul & Gish, “Local Alignment Statistics,” in Multiple Alignment and Phylogenetic Trees (Volume 266), 1996, pp. 460-480.
[26] Needleman & Wunsch, “A General Method Applicable to the Search for Similarities in the Amino Acid Sequence of Two Proteins,” J. Mol. Biol., vol. 48, pp. 443-453, 1970.
[27] Myers & Miller, “Optimal Alignments in Linear Space,” Comput Appl Biosci (CABIOS), vol. 4, no. 1, pp. 11-17, 1988.
[28] Abeyrathne et al., “Functional Characterization of WaaL, a Ligase Associated with Linking O-Antigen Polysaccharide to the Core of Pseudomonas aeruginosa Lipopolysaccharide,” J. Bacteriol., vol. 187, no. 9, pp. 3002-3012, 2005.
[29] University of Alberta, U.S. Pat. No. 9,238,830 (Jan. 19, 2016).
[30] Musumeci et al., “In Vitro Activity of Neisseria meningitidis PglL O-Oligosaccharyltransfemse with Diverse Synthetic Lipid Donors and a UDP-activated Sugar,” J. Biological Chemistry, vol. 288, no. 15, pp. 10578-10587, 2013.
[31] Sun et al., “Design and production of conjugate vaccines against S. Paratyphi A using an O-linked glycosylation system in vivo,” npj Vaccines, vol. 4, pp. 1-9, 2018.
[32] Pichichero, “Protein carriers of conjugate vaccines,” Human Vaccines & Immunotherapeutics, vol. 9, no. 12, pp. 2505-2523, 2013.
[33] Ge et al., “The C-Termainal Domain of AcrA is Essential for the Assembly and Function of the Multidrug Efflux Pump AcrAB-TolC,” J. Bacteriology, vol. 191, no. 13, pp. 4365-4371, 2009.
[34] Glaxosmithkline Biologicals SA WO/2017/067964 (Apr. 27, 2017).
[35] Avci et al., “A mechanism for glycoconjugate vaccine activation of the adaptive immune system and its implications for vaccine design,” Nature Medicine, vol. 17, no. 12, pp. 1602-1610, 2011.
[36] Dagan et al., “Glycoconjugate vaccines and immune interference: a review,” Vaccine, vol. 28, pp. 5513-5523, 2010.
[37] FORSGREN, Arne; European Pat. No. EP0594610 B1 (Sep. 2, 1998; related to WO 91/18926).
[38] Prymula et al., “Pneumococcal capsular polysaccharides conjugated to protein D for prevention of acute otitis media caused by both Streptococcus pneumoniae and non-typable Haemophilus influenzae: a randomised double-blind efficacy study,” The Lancet, vol. 367, pp. 740-748, 2006.
[39] Smithkline Beecham Biologicals S.A. WO/2000/056360 (Sep. 28, 2000).
[40] Uchida et al., “Mutation in the Structural Gene for Diphtheria Toxin carried by Temperate Phage β,” Nature: New Biology, vol. 233, pp. 8-11, 1971.
[41] Pappenheimer et al., “Diphtheria Toxin,” Ann. Rev. Biochem., vol. 46, pp. 69-94, 1977.
[42] Rappuoli, “Isolation and Characterization of Corynebacterium diphtheriae Nontandem Double Lysogens Hyperproducing CRM197,” Applied and Environmental Microbiology, vol. 46, no. 3, pp. 560-564, 1983.
[43] Neubauer & Helting, “Structure of Tetanus Toxin: the arrangement of papain digestion products within the heavy chain-light chain framework of extracellular toxin,” Biochiica et Biophysica Acta, vol. 668, pp. 141-148, 1981.
[44] Rubins et al., “Distinct roles for pneumolysin's cytotoxic and complement activities in the pathogenesis of pneumococcal pneumonia,” American Journal of Respiratory and Critical Care Medicine, vol. 153, no. 4, pp. 1339-1346, 1996.
[45] Walker et al., “Molecular cloning, characterization, and complete nucleotide sequence of the gene for pneumolysin, the sulfhydryl-activated toxin of Streptococcus pneumoniae,” Infect. Immun., vol. 55, no. 5, pp. 1184-1189, 1987.
[46] Mitchell et al., “Expression of the pneumolysin gene in Escherichia coli: rapid purification and biological properties,” Biochimica et Biophysica Acta, vol. 1007, no. 1, pp. 67-72, 1989.
[47] Mitchell et al., “Comparison of pneumolysin genes and proteins from Streptococcus pneumoniae types 1 and 2,” Nucleic Acids Research, vol. 18, no. 13, p. 4010, 1990.
[48] Glaxosmithkline Biologicals S.A. WO/2004/081515 (Sep. 23, 2004).
[49] Glaxosmithkline Biologicals S.A. WO/2006/032499 (Mar. 30, 2006).
[50] Paton, et al., WO 1990/006951 (Jun. 28, 1990).
[51] Berry et al., “Comparative virulence of Streptococcus pneumoniae strains with insertion-duplication, point, and
deletion mutations in the pneumolysin gene,” Infection and Immunity, vol. 67, no. 2, pp. 981-985, 1999.
[52] Patent WO 1999003884.
[53] Low et al., “Optimisation of Signal Peptide for Recombinant Protein Secretion in Bacterial Hosts,” Appl. Microbiol. Biotechnol., vol. 97, pp. 3811-3826, 2013.
[54] Yoon et al., “Secretory Production of Recombinant Proteins in Escherichia coli,” Recent Patents on Biotechnology, vol. 4, pp. 23-29, 2010.
[55] Tian et al., “Predicting Synonymous Codon Usage and Optimizing the Heterologous Gene for Expression in E. coli,” Sci. Reports, vol. 7, no. 9926, pp. 1-9, 2017.
[56] Chin et al., “Codon Optimization OnLine (COOL): a web-based multi-objective optimization platform for synthetic gene design,” Bioinformatics Applications Note, vol. 30, no. 15, pp. 2210-2212, 2014.
[57] Hilterbrand et al., “CBDB: The codon bias database,” BMC Bioinformatics, vol. 13, no. 62, pp. 1-7, 2012.
[58] Codon Usage Database, available at http://WorldWideWeb(www).kazusa.or.jp/codon/, KAZUSA DNA RES. INST.; last visited Jul. 30, 2018.
[59] Codon Optimization Tool, available at http://WorldWideWeb(www).idtdna.com/CodonOpt, INTEGRATED DNA TECHNOLOGIES; last visited Jul. 30, 2018.
[60] The Codon Bias Database, available at http://WorldWideWeb(www)cbdb.info (as described in Hilterbrand et al., 2012 BMC Bioinformatics 13:62, 7 total pages).
[61] Thermo Fisher Scientific, Protein Preparation Handbook, 2017.
[62] Thermo Fisher Scientific, Fusion Protein Purification Resins, 2013.
[63] Fisher et al., “Production of Secretory and Extracellular N-Linked Glycoproteins in Escherichia coli,” Applied and Environmental Microbiology, vol. 77, no. 3, pp. 871-881, 2011.
[64] Klein et al., “Design and Characterization of structured Protein Linkers with differing Flexibilities,” Protein Engineering, Design & Selection, vol. 27, no. 10, pp. 325-330, 2014.
[65] Rosano & Ceccarelli, “Recombinant Protein Expression in Escherichia coli: advances and challenges,” Frontiers in Microbiology, vol. 5, no. 172, pp. 1-17, 2014.
[66] ETH ZÜRICH WO/2003/074687 (Sep. 12, 2003).
[67] ETH ZÜRICH WO/2006/119987 (Nov. 16, 2006).
[68] Feldman et al., “Engineering N-linked protein glycosylation with diverse O antigen lipopolysaccharide structures in Escherichia coli,” PNAS, vol. 102, no. 8, pp. 3016-3021, 2005.
[69] Keys & Aebi, “Engineering Protein Glycosylation in Prokaryotes,” Current Opinion in Systems Biology, vol. 5, pp. 23-31, 2017.
[70] Sambrook & Russell, Molecular Cloning: A Laboratory Manual 3rd Edition, Cold Spring Harbor, N.Y.: CSHL Press, 2001.
[71] Jones Day, WO/2014/037585 (Mar. 13, 2014).
[72] Kowarik et al., “N-Linked Glycosylation of Folded Proteins by the Bacterial Oligosaccharyltransferase,” Science, vol. 314, no. 5802, pp. 1148-1150, 2006.
[73] Max-Planck-Gesellschaft zur Förderung der Wissenschaften, WO/2015/004041 (Jan. 15, 2015).
[74] Max-Planck-Gesellschaft zur Förderung der Wissenschaften E.V., WO/2016/091399 (Jun. 16, 2016).
[75] Max-Planck-Gesellschaft zur Förderung der Wissenschaften E.V., WO 2016198170 (Dec. 15, 2016).
[76] Wyeth L L C, US Pregrant Pub. No. 2007/0184072 (Aug. 9, 2007).
[77] University of Rochester, U.S. Pat. No. 4,673,574 (Jun. 16, 1987).
[78] Merck & Co. Inc., EP 0161188 B1 (Apr. 3, 1991).
[79] SCLAVO SPA, EP0208375 B1 (Dec. 11, 1991).
[80] American Cyanamid Co. EP0477508 (Jul. 12, 1995).
[81] U.S. Government as represented by the Secretary Of The Army, WO/1993/015760 (Aug. 19, 1993).
[82] Henry M. Jackson Foundation, WO/1995/008348 (Mar. 30, 1995).
[83] Lees, A., WO/1996/029094 (Sep. 26, 1996).
[84] Chu et al., “Further studies on the Immunogenicity of Haemophilus influenzae Type b and Pneumococcal Type 6A Polysaccharide-Protein Conjugates,” Infection and Immunity, vol. 40, no. 1, pp. 245-256, 1983.
[85] Leyva et al., “Rapid and sensitive anthrone-sulfuric acid assay in microplate format to quantify carbohydrate in biopharmaceutical products: method development and validation,” Biologicals, vol. 36, no. 2, pp. 134-141, 2008.
[86] Dische & Shettles, “A specific color reaction of methylpentoses and a spectrophotometric micromethod for their determination,” J. Biol. Chem., vol. 175, no. 2, pp. 595-603, 1948.
[87] Pogue et al., “Production of pharmaceutical-grade recombinant aprotinin and a monoclonal antibody product using plant-based transient expression systems,” Plant Biotech. J., vol. 8, pp. 638-654, 2010.
[88] Novartis A G, WO/2007/109812 (Sep. 27, 2007).
[89] Novartis A G, WO 2007/109813 (Sep. 27, 2007).
[90] Shirota & Kilnman, “Recent Progress Concerning CpG DNA and its use as a vaccine adjuvant,” Expert Rev. Vaccines, vol. 13, no. 2, pp. 299-312, 2014.
[91] The University of Iowa Research Foundation, WO/1996/002555 (Feb. 1, 1996).
[92] Cambridge Biotech Corp., U.S. Pat. No. 5,057,540 (Oct. 15, 1991).
[93] Stoute et al., “A Preliminary Evaluation of a Recombinant Circumsporozoite Protein Vaccine against Plasmodium falciparum malaria RTS, S Malaria Vaccine Evaluation Group,” N. Engl. J. Med., vol. 336, no. 2, pp. 86-91, 1997.
[94] Afiris A G, US Pre-grant Pub. No. 2015/0093431 (Apr. 2, 2015).
[95] Novartis A G, WO/2011/027222 (Mar. 10, 2011).
[96] Novartis A G, Patent US Pre-grant Pub No. 2012/0237546 (Sep. 9, 2012).
[97] Buonsanti et al., “Novel Adjuvant Alum-TLR7 Significantly Potentiates Immune Response to Glycoconjugate Vaccines,” Sci. Rep., vol. 6, p. 29063, 2016.
[98] Sabroe et al., “Toll-Like Receptors in Health and Disease: Complex Questions Remain,” J. Immunol., vol. 171, no. 4, pp. 1630-1635, 2003.
[99] Smithkline Beecham Biologicals S.A., Patent WO/1995/017210 (Jun. 29, 1995).
[100] Reilly, William J Jr, “Chapter 36: Pharmaceutical Excipients,” in Remington Essentials of Pharmaceutics, London, Pharmaceutical Press, 2012, pp. 683-704.
[101] Wacker et al., “N-linked glycosylation in Campylobacter jejuni and its functional transfer into E. coli,” Science, vol. 298, no. 5599, pp. 1790-1793, 2002.
[102] Shepherd et al., “Comparison of O-antigen gene clusters of Escherichia coli (Shigella) sonnei and Plesiomonas shigelloides O17: sonnei gained its current plasmid-borne O-antigen genes from P. shigelloides in a recent event,” Infect. Immun, vol. 68, no. 10, pp. 6065-6061, 2000.
[103] Hartley et al., “Biochemical Characterization of the O-Linked Glycosylation Pathway in Neisseria gonorrhoeae Reponsible for Biosynthesis of Protein Glycans Containing N,N′-Diacetylbacillosamine,” Biochemistry, vol. 50, no. 22, pp. 4936-4948, 2011.
[104] Anonsen et al., “Characterization of a Unique Tetrasaccharide and Distinct Glycoproteome in the O-Linked Protein Glycosylation System of Neisseria elongata subsp. glycolytica,” J. Bacteriology, vol. 198, no. 2, pp. 256-267, 2016.
[105] Endo and Koizumi, “Large-Scale Production of Oligosaccharides Using Engineered Bacteria” 2000 Curr. Op. in Structural Bio., vol. 10, pp. 536-541 (also in HANDBOOK OF CARBOHYDRATE ENGINEERING 1ST ED. 2005).
[106] Clausen et al., “Chapter 56: Glycosylation Engineering” in ESSENTIALS OF GLYCOBIOLOGY 3 Ed. 2017 (available at https://www.ncbi.nlm.nih.gov/books/NBK453027/).

1-24. (canceled)

25. A modified carrier protein comprising a carrier protein that comprises at least one GlycoTag, wherein the at least one GlycoTag is a Neisseria gonorrhoeae PglL GlycoTag (NgGlycoTag), Neisseria lactamica PglL GlycoTag (NlGlycoTag), or Neisseria shayeganii GlycoTag (NsGlycoTag), or combinations thereof.

26. The modified carrier protein of claim 25, wherein the at least one NgGlycoTag consists of a peptide sequence that is 12 to 30 amino acids long and comprises therein the sequence SEQ ID NO: 147.

27. The modified carrier protein of claim 25, wherein the at least one NlGlycoTag consists of a peptide sequence that is 12 to 35 amino acids long and comprises therein the sequence SEQ ID NO: 151.

28. The modified carrier protein of claim 25, wherein the at least one NsGlycoTag consists of a peptide sequence that is 12 to 31 amino acids long and comprises therein the sequence SEQ ID NO: 164.

29. A modified carrier protein comprising a carrier protein that comprises at least one GlycoTag, wherein the at least one GlycoTag is a Neisseria meningitidis PglL GlycoTag (NmGlycoTag) consisting of a peptide sequence that is 12 to 19 amino acids long and comprises therein the sequence SEQ ID NO: 142.

30. The modified carrier protein of claim 25, wherein the carrier protein is selected from the group consisting of cholera toxin b subunit (CTB), tetanus toxoid (TT), tetanus toxin C fragment (TTc), diphtheria toxoid (DT), CRM197, Pseudomonas aeruginosa exotoxin A (EPA), C. jejuni Acriflavine resistance protein A (CjAcrA), E. coli Acriflavine resistance protein A (EcAcrA), and Pseudomonas aeruginosa PcrV (PcrV).

31. A modified carrier protein, characterized by a Pseudomonas aeruginosa exotoxin A (EPA) carrier protein comprising at least one Neisseria meningitidis PglL GlycoTag (NmGlycoTag), wherein the at least one NmGlycoTag is located at, with respect to SEQ ID NO: 1, residue A14, D36, Q92, G123, E157, A177, Y208, N231, E252, R274, A301, Q307, A365, S408, T418, A464, A519, G525, A533, S585, K240, or A375, or combinations thereof.

32. The modified carrier protein of claim 31, wherein the at least one NmGlycoTag consists of a peptide sequence that is 12 to 29 amino acids long and comprises therein the sequence SEQ ID NO: 142.

33. A nucleic acid molecule comprising a nucleotide sequence that encodes the modified carrier protein of claim 25.

34. A vector comprising the nucleic acid molecule of claim 33 and wherein the modified carrier protein nucleotide sequence is operatively linked to a polynucleotide sequence encoding a periplasmic signal sequence, optionally further comprising a nucleic acid molecule that comprises a nucleotide sequence encoding a Neisseria meningitidis PglL (NmPglL) Oligosaccharyltransferase (OTase), Neisseria gonorrhoeae PglL (NgPglL) OTase, Neisseria lactamica 020-06 (NlPglL) OTase, Neisseria lactamica ATCC 23970 PglL (Nl_{ATCC23970}PglL) OTase, or Neisseria gonorrhoeae F62 PglL (Ng_{F62}PglL) OTase.

35. A gram-negative bacterial host cell comprising the vector of claim 34.

36. A conjugate comprising the modified carrier protein of claim 25 and one or more other molecules.

37. A method of making a conjugate, comprising contacting a PglL OTase and a PglL Glycan Substrate in the presence of the modified carrier protein of claim 25; thereby making the conjugate, optionally then isolating the conjugate.

38. An immunogenic composition comprising the modified carrier protein of claim 25 covalently attached to one or more immunogenic glycans.

39. The immunogenic composition as in claim 38 for use in inducing an antibody response in a mammal.
